SEQUENCE LISTING (79 sequences)

SEQ ID NO: 1 - DNA, 758 bp, Homo sapiens
  misc_feature (1)...(758): n = A,T,C or G
cactagtaacggccgccagtgtgctggaattcggcttagcgtggtcgcggccgaggtacatttggttaaacattttataaaaattcaagcaaaatttaaaacacagccaatttcaacatttctacaaatttaaccagcattattttggtcaaatccacctttagctgaattaatgagaacaaggacaagggacattgaagtattctcctcaccacaatggaggtttttctcactttccacactgcatagccactacatggaaactcccagaatgatgagcaattcagtcatgctctacacttttcttttcaatgaaaagtgatgtatcttcttgttttgcttttgacaaaatatgaactaaatacaagtctataataacaagaatccaagagtagctcatacacaagtcaactatgtgtatgtttttattatttaaagcccaccattcagcttatctgtctaaagagatgataacaaacaatttgtttctttttcactaatggatactggtgancagtcagtggttaaaanagggaaggtccaagacagttgttnttaatccagtccattgcccacatttaactttttcaactttaagaattttgcttttgcctttgcctaaaaagggnaaaacttgtggnagntaaacggncaaaccctttggctttgaaagttaatncnggatgatccaggttngtcctggccangtttctaaaagcccctggtttttatttgacgngggccggtaatccaaaggg

SEQ ID NO: 2 - DNA, 739 bp, Homo sapiens
  misc_feature (1)...(739): n = A,T,C or G
gtaacggccgccagtgtgctggaattcggcttagcgtggtcgcggccgaggtacaggcgctggacagtataggcatgcatctccacactattagttattaactggattaaactggggactgcatcatcacgaacataacttcctgccgttgtcaaaacacgcataattgtgtctatatgccatcgtttggaaggtgcatacttttctgcagcaagaaagattccagatgcacagtctgctttaaattctggctcacacgaatccagaaaataaagtaattctttcatcatgcctcggatattattcccatttaccagggcaaaactcaattccattgcacgccgttttattgagacatccaaatctttaagacagtccacaattgtgcttctgtgcctctgtacctgcccgggcggccgctcgaagccgaattctgcagatatccatcacactggcggccgctcgagcatgcatntagagggccaattcgcctatagtgagtcgtattacaaatcactgggcgncgntttacacggtngngactgggaaaccctggngtccccacttaatngcntgnagnaaatcccctttngccgnttggcgtaaaacgaaaaggccccaccgntcgctttncaacagttgccaccntgatggnaatggccccccttgacgggcataagcgcggnggnggggggtaccccaanggaccgtcattgcaggcctagcccgtctttgtttt

SEQ ID NO: 3 - DNA, 823 bp, Homo sapiens
  misc_feature (1)...(823): n = A,T,C or G
agnnnnnnaggnnntnnnnnnnttnnnnncnntctnggnaccnagctcggatccctagtaacggccgccagtgtgctggaattcggcttagcgtggtcgcggccgaggtactgtaattctttgattgggatagtggaaactctagccaaaaaaaaaaaatgaacacagaagtacagtaaataatatggtgaagtctaccccaatatccctagggtggttgtggttagggctataacgtagtatcaatatgaagatattacagcatgtatttaaaagcaattgactattaggtatcagacttagtgttatgatacatttgtctaatgaaatgaaatctgttttcagaaataatctttaaatacaatggaatttattgagcatctactattatatttcatccatcacttttaaattttttctttgggaggaacacttataaactcatgatatggataacagacctgtggaaattaatgtaagccctaagcagcttagttagacattgaatctaggtctgcctctaccctgcagtctaccagntcaagtagctttaccaggtcaagtccttanaaaataaccataaccatccaagggcagngcagttattaacnaggtgtncctgcccggccggccgttcgaaagccgaattttgcaanntttccatcacactggcggccgttcgancatgcatttaaagggccaattcgncctntgggngtcgaatacaatcactggccgcgtttacaacgtcgngctgggaaaacctngcgtncccanttaatcgcttggncnatnccctttcccagctggngaatancnaaan

SEQ ID NO: 4 - DNA, 756 bp, Homo sapiens
  misc_feature (1)...(756): n = A,T,C or G
ctagtaacggccgccagtgtgctggaattcggcttagcgtggtcgcggccgaggtacctctgacaatgaagcctaaattgtttacgttggagagggagtgggagataagctaaagaatgtgatttgtcttccatttgaagataccagggaaaagtcttgtcaagtagcaggccaccggtgtctagtgtagaggagacgatttctgtcgatagagagcaaagccagccaggcaaacgaacccgtaagccgcctgagggacagacaggcataaaacctaatgaatgggtaggtagactttgggcaaggctaattgcagcaagctgtgttcagttccccggcctctgttaagtagtcatttttatacgatctgtcggagggaagaggaaatagacccacacggtaaatgaacttgtggtgtttagctaggaagtggagtttgctctaagtagctctggtagcaagactccagggagcacatggcaggcggactcatctgagtatctgtgctnanggagttgtgttaactgttgaaacacttanaaaattggtntanaaaaaaataccttncccggcnggccgttcgaaagccgaatttttgcanatttccatacactggcggccgttgacatgcatntaaagggccaattcncctatangagtcgattacaattnctggccgcggtttacacgttgggatgggaaaacctgcgtcccaactaattgcttgggnnatcccnttgccactgctaatacnaa

SEQ ID NO: 5 - DNA, 736 bp, Homo sapiens
  misc_feature (1)...(736): n = A,T,C or G
ggccgccagtgtgctggaattcggcttcgagcggccgcccgggcaggtacattacattttatgaagggaaacaaagagttaattacaagatgcaaaaagataagaaagagacaaactacagcgtgagtattgttcagaggtagtaagtgagcactctaagctacagaacatgttgaaattctattggtttgtatgagttcgcatgaggtaataaagctgtgttttgattggtgggatgtataaatactcttttnctacactatatacaacactccagagagcagggccttttgcagatgcccagaggacgtagcaaaccttgacaagtctgtgcagcacccagtgcacaccgtaaaaccccagacggatcaatctcgttttcacctctatcagcagcaagtatcttcgtctctcagaacaatctggatccataattttattaccgagcagactctggggcactggtgctttcagagagagaaacgatgctaccagtctttgactaattntacataagagatctgaacccaaacgtgcgaggagntttttaaccccaccatctttaacngggttggcaaacagttnttnttccacctcctttgtgatttgaaagaaagacatccagtcagtatggaatntgcaaaagtcaattaggngnggggatttgcattgtttacggtgnggttanngttttggcctggccggacccttagccgatttgannatcatccctgng

SEQ ID NO: 6 - DNA, 769 bp, Homo sapiens
  misc_feature (1)...(769): n = A,T,C or G
gagctcggatccactagtaacggccgccagtgtgctggaattcggcttagcgtggtcgcggccgaggtacccaatgaaggatccgatgaatggctggccagttactcagagtaatatgtttatgagttaaatggattgcataacggcaccacttttcctttgtcactgtggtggataaatgacaaagtagattccaatctcctgtatagaaaggtggttacccaatagcctcactggtggcttgctagcagagcctcaagttgcacctctgtggctttctgtaaattcgagtcctacagccacttcgagaacgctgcaatccccaaggcccacttggtccatgctgaagaagcggctgtgaccccaatcctgcctctcttttgctcttcagtattacttactggaggaatatgtaatattctaggtcaagtctaggagaggctcatcctaaaaaaaaaattgcaaagtcttttctcaattaaaacaaaaaaagcctcaagtacctgcccggcgggcgctcgaagcccnaatnttnagatattcatacaattgcnggcggttgagccttgcttttagangggccaaattggccctatagngngtngantacaattcactggccggcgtttacaacgtcnngactgggaaaccccggggttncccacttaatnccttgagganatcccctttntcngctggngtaaaacnaaaaggcccaccgntgcctttccanaatgcccacctatgggaangaccccccttacggcnt

SEQ ID NO: 7 - DNA, 817 bp, Homo sapiens
  misc_feature (1)...(817): n = A,T,C or G
gnnnnnntnnnagnnnnnttnnnnnttnnntnnnngctnggtaccgagctcggatccactagtaacggccgccagtgtgctggaattcggcttagcgtggtcgcggccgaggtactcagtgtaaggctattatcgtttttcatacataaaattttctagctctgtaacacaatgcaatttttaatccattcaagtaagttcaaccccaaagttgccgcttcccagcattaagacatgcacccacccctcttctaagattttctaaaacttgtatttcggggagaaagacctcttttaaaaaataatcccaattagtgggagagtaaatggctgacattagtagcaaaaccttagttatctgaaaataacatattggaaatgagacattattaggattttaaacaaacaatagcatttagacataaagtaggaagcaaaatacagtaaacagaaatagtgtagccaaatatgcattctcttcagctacactagagtggaccttgcttagtacctgcccgggcgggcgctcgaaagcccgaattctgcagatttcatcacactggcgggcgnttcancctgcatttagangggcccattcgccctntaggggggtcgtattcaaatcactngccgcggtttacaaccgtcgngactgggaaaaccctgggggttcccacttaatcgccttggagcacatcccttttggcagctggngtatancgaaaggcccgaccgacgccttncaacnntgcncacctgaaggngaaggacccccctgtanggnncntaacgcgggggggnggggtncccncggnct

SEQ ID NO: 8 - DNA, 755 bp, Homo sapiens
  misc_feature (1)...(755): n = A,T,C or G
cactagtaacggccgccagtgtgctggaattcggcttagcgtggtcgcggccgaggtactacatctgaggattccttggtcctttacaatagagtggctgttcaaggagatgtggttcgtgaattaaaagccaagaaagcaccaaaggaagatgtagatgcagctgtaaaacagcttttgtctttgaaagctgaatataaggagaaaactggccaggaatataaacctggaaaccctcctgctgaaataggacagaatatttcttctaattcctcagcaagtattctggaaagtaaatctctgtatgatgaagttgctgcacaaggggaggtggttcgtaagctaaaagctgaaaaatcccctaaggctaaaataaatgaagctgtagaatgcttactgtccctgaaggctcagtataaagaaaaaactgggaaggagtacctgcccgggcggccgctcgaaagccgaattctgcagatatccatcacactggcggncgctcgagcatgcattntaganggcccaattcgccctatagtgagtccgantacaattcactggccgcggtttacaaccgtcgtgactgggaaaccctggcgttacccaacttaattgccttgngcacatncccctttcgccagttgcgtaatancnanaggncccaccgnccccttccacaattcccacctgatggnnatggnccccccttacgggcataagcccggggggtgnggttcccaaggnaccctccttct

SEQ ID NO: 9 - DNA, 766 bp, Homo sapiens
  misc_feature (1)...(766): n = A,T,C or G
tcggatccctagtaacggccgccagtgtgctggaattcggcttcgagcggccgcccgggcaggtacttatctggcagcactgagatgctcagttcaaattcttggccatcctgaaagggcatgttcttggattccacctgctgcttccaggccccatactcacggctgttcatgaccacacgacgaccaaagcacacttggaaatggaagacaatgtctgattcctccttcatctcagtgtggaaatccacctgcagatatggttcattcaagaaacagacaagtggtcgccctttgattgtcacagtagaaccagtagacaaagaggcagcctctgtgtatggcacgggtagcagggacattgttgtctccttctgggtggctcttctgaattgtgtccagacttccccgcgtacctcggccgcgaccacgctaagccgaattctgcagatatccatcacactggcggccgctcgagcatgcatntagagggcccaatttcgcctatagtgagtcgtantacaattcactggccgtcgttttacaacgtcgngactgggaaaaccctggcgttcccaacttaatcgcttgaacacatccccttttcgccagttgngtaataacgaaanaggcccgcaccgncgcctttccaacagtgcncancctgaatggcgaatggacccccctntagcgggcattaaacgcgggggtgtggtggtacnccanngtnaccgttcanttgcaggcctannccgtcntttntttt

SEQ ID NO: 10 - DNA, 743 bp, Homo sapiens
  misc_feature (1)...(743): n = A,T,C or G
ctagtaacggccgccagtgtgctggaattcggcttagcgtggtcgcggccgaggtaccttagctgtgaggggggctgctgaaggcatggaggacagtaatgacggctgtgcctccttggatggtggagacctcagaaatggtctgcaacacaggcactaggtcagtttcccgcccttagctgaatgcagatggatgctgggagatggacatggtgagatggtgtggggaggatgttagaaagctggaggtgtttatcacttctcaaggagctctgaatatagggggaaaaaaacaacaaaaaaaagcccagggagaaagcaccattaaggatatacctatgccagcttctatagctgcacctgctttattggcaggtcacttgccacaattgcatctgccctctaaactttttaattttcacactgtatcctagacatgaatgcataaaatacagtgtctggcgggataatgcagagaggtcctgcccgggcggccgcaagcccgaattctgcaagatatncatacactggcgggccgttcgacatgcatttagaggcccaattcgccctatagtgagtcgnntacaatttactgccncgttttacaacgtcgtgactggnaaaccctgcgttacccacttaatcgnttgnagnnatcccntttnccagttgctatagcnaanagccccncgncgccttccaacattgtcacttatgggatggacccccttacggncataacc

SEQ ID NO: 11 - DNA, 825 bp, Homo sapiens
  misc_feature (1)...(825): n = A,T,C or G
agnnnnagnnnnnagnntnnnnnnnttnnnncnnnctnggaccggctcggatccactagtaacggccgccagtgtgctggaattcggcttagcgtggtcgcggccgaggtacaagtgaaatatttaaataggaatctgaaacaaaacgaattcaatctgatcaaatccacaattaattgaagttttcattttattcaattgtgaataaaatagcagacactgtttcatccaataagccaatgatatcagcttaggagaaatgatctgcctggcttgtgcaagacaagaacagttaccttctgctgaaaggatgtgagttttcaaatttggttttcatgtcatagtttccaaataaatgaaccgctccaccaaaaacgacagtctagttgctacctatcttccagggtttcatcatacagccatttataagcacaatctttggaacgctctatagtttcaaaaagtgttttgaagtcttgagggtgcatgttaactactttctcatcctcctgttttcctatttgcatcaatatatctctcgcatcaaggnggagtcaatgaaccgtaagtggtctattacaggtgggcgctgcgccctcccgaaacaagtgctttaacaaggtgaaaaaatatntttngntcatanggngtnaggtcttggnaaataaattttgacgaggaatcatttgaataananacagcttttngtngnaattggctaanaattccctacncttngggtttccaccggnccttccggtgttntttnaaatgatgccangctcatgggcgaacctaatctccaagtaatgccagnnacnctna

SEQ ID NO: 12 - DNA, 761 bp, Homo sapiens
  misc_feature (1)...(761): n = A,T,C or G
tcggatccctagtaacggccgccagtgtgctggaattcggctttcgagcggccgcccgggcaggtacatctccggtgtccctgggacagggaagactgccactgttcatgaagtgatacgctgcctgcagcaggcagcccaagccaatgatgttcctccctttcaatacattgaggtcaatggcatgaagctgacggagccccaccaagtctatgtgcaaatcttgcagaagctaacaggccaaaaagcaacagccaaccatgcggcagaactgctggcaaagcaattctgcacccgagggtcacctcaggaaaccaccgtcctgcttgtggatgagctcgaccttctgtggactcacaaacaagacataatgtacctcggccgcgaccacgctaagccgaattctgcagatatccatcacactggcggccgctcgagcatgcatctagagggcccaattcgccctatagtgagtcgtattacaattcactggccgtcgtttacaacgtcgtgactnggnaaaccttggcgttncccaacttaatcgccttgangacatncccctttcgccagctggcgtaatagcgaagaggccgcaccgatcgcctttccaacagttgcgcancctgaatggggaatggacgcgcctgtaccggccattaacgcggngggtgnggnggttcccncaggtgaccgttcanttgcagngcctacgnccgtctttngttttttcttctttttgccgttgccggtttcccgta

SEQ ID NO: 13 - DNA, 766 bp, Homo sapiens
  misc_feature (1)...(766): n = A,T,C or G
ggtaccgagctcggatccctagtaacggccgccagtgtgctggaattcggcttagcgtggtcgcggccgaggtacgcgggacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcangcttnctggcagaagatggtgactggagtgggcagtgncctgtccttcanataccacttgagctcacttgccatgatgcaaaactttcaaggataggctttattntgcaagcaatcaaataataaatctnttntgtnaaaaaaaaaaaaaaaaaaaaaaaaagtccttgccggcggccgtcaaaccgaatttganattcatacatgngggcgtcacatcattaaaggccaatcnctnanggtgnttaaa

SEQ ID NO: 14 - DNA, 732 bp, Homo sapiens
  misc_feature (1)...(732): n = A,T,C or G
ggccgccagtgtgctggaattcggcttagcgtggtcgcggccgaggtacacagattttaaaagcattttttaaatttatttttattttttccagatttttttaggatataattgacaagtaaaaaatgaatatatttaaggtgcacaatgcgatgttttggtatacgtggacattgtgaagtggttgcaacaattaagttaaataacatatctgtcacctcacatagttacctttgtgtgtgtgtgtgtgtgtttgatgagcataaaatctatctagcagcaaaggcattttctaaagtccagaaaatgccactaatatatgcattaataatatacaaaaaaccagtaactaactttattatgttaatttatcttttcttttctctgntgaactatttatttacatacctaaactctactcaaactgctgttgtnaaaaacaaggaggaattagtttctcttnatctctaacaccagaagaagatccattttgnagtcttattgctctgntgaatgactgtctcctggtttgnggcagactggaagcaagagtctntctgtttggccacagtcctgcccgggcgggcgtcgaaagcccgaatttgcagattccatcnactggcggccgtcgagctgctttagagggccaattcgccttagggaggggatacaatcactggcgcgtttcacgcgggctggnaacctngggtnccactaattgctgaccatccn

SEQ ID NO: 15 - DNA, 754 bp, Homo sapiens
  misc_feature (1)...(754): n = A,T,C or G
tcggatccactagtaacggccgccagtgtgctggaattcggctttcgagcggccgcccgggcaggtaccttacttagcagagcactttgcaaacatattacttattagcagagctctttgtagaccttccacatctggctgtcagatcttaaggttgtgaatttaggctccagttatattcactggagagcataatcccacacgggttatttataaatacagagcctctgattggacgggctcctgccaagaactagtaatacccttgttttaaaatcttcacaaggtaagacttaaaaagccaaccaaacaaattgctctccattctacttttaattgggccaaacagcatatgctacagtagtaacatgtttttcggagagtgtaaaaaactctgtttacatttgcctcctccgtgggttgatcgaaaatgtataaaactgactgcttctcgccagcctcagacaagaaagagtgagttgctggtcctcggccgcgaccacgctaagccgaattctgnagatatncatcacactggcgggncgntcgagcatgcatntagagggccaattcgncctatagtgagcgtattacaattcactgggcgcgntttacaacgtcgggctgggaaaccctggcgttcccacttaatcgctgnagnacatccctttgcagtggggtaaaacgaaaggccgacgatgncttccaacattgccanctgatgggaatggcnccctnagcgncataacgcggg

SEQ ID NO: 16 - DNA, 753 bp, Homo sapiens
  misc_feature (1)...(753): n = A,T,C or G
ctagtaacggccgccagtgtgctggaattcggctttcgagcggccgcccgggcaggtaccaaccaaaatctcgtattggcagatcttgacaggctggacctgcaagatgtgacttgaattttaaccatttattacataatctctagtgatcatgcatctagttatcataagaaataatttaaaaggttttgttgctgaaagcagtaagtggtgcagtaaaatacttaagttattcaaagaatgttatctttcttgcaagagtaatttaagcacatgggaaagattctagactttttgtttcttgcaacaacagtgccctctgctgctagaaacctttttctacttactatcatttttattgtggcttgagctcagtcaatctggtgcagatgaggctggacaactactaaccaataaaatcaggagtttgtacctcggccgcgaccacgctaagccgaattctgcagatattcatcacactggcgggcgctcgagcatgcatttaagagggcccaattcgcctatagtgaggcntattacaaattnatggccgtcgttttacaccgcgtggntggggaaaaccctggcggtacccaacttaatggnttggagaacattccccttttcgccanttgggtaatacccnaaagggccccaccgatcgcctttccaacatttgcncacctgatggnaatggnccccccttacgggcattaagccggggggtgtggnggtcccccacggncntccttn

SEQ ID NO: 17 - DNA, 744 bp, Homo sapiens
  misc_feature (1)...(744): n = A,T,C or G
ctagtaacggccgccagtgtgctggaattcggcttagcgtggtcgcggccgaggtactccaacaagacatgcataccattggtgcctaagatttttttacagttgtgtttttatacagaaattctttgtagaaattactattttttgttaaagattgtttatatgcttacaaagatttctcaggaagacagcagagcagaggaatctatatagatgtatgcacagacctgtctgtatgctgaactttgttaaaaatatctgccagttattaaaaagcacagtttaaatggggtggggttaaagttcaggtaagtaagttagagagaaaacattgtatgatcagctcctgcacttgatctatctttggcttcccaaacagtaactcactccaggccaagtgtgccttagcacgagtgaccacagtttaatagaccacacacatcgtttaaacctgctcttggtcattggnaattacactgaacaaagtgcaattactgnngaacagtttatttttattaaaaccttgnctgacaaaaggggncctcacattgtngaccctgggngccctacanaggntagccttttctttaaggnttaaccnggggctggtggttttttttttgaaagagttaaggncnaaaattaatttgtngttgnnaaaaaaaaaattccgtctttcggntgantgntttgcangtcanaaaaanggacctgccnggnggcgtnnaaccaatttggaattcn

SEQ ID NO: 18 - skipped

SEQ ID NO: 19 - DNA, 763 bp, Homo sapiens
  misc_feature (1)...(763): n = A,T,C or G
ctagtaacggccgcagtgtgctggaattcggctttgagcggccgcccgggcaggtacaggaagcacaccttgatactgaggccatttagagcaatttgtttaatgagtgggttcagccatcaccaccagtggccacctgttcaataaaattgcctagcaccacagtgggagagtcatctacaccccaaggtgagctgtaagactggccaattctttgaagaccttttttctgttgggagaatggaaagcagaaagggaagccaggctctttaatgttccaagtcaagctgtgcctcacacaaggctctctttttagctactaagtaacatactcaagactttgtatctgtgcagcattttatggaaatcactggtgacctataggatattactaaattatttcaatagctttatttttttcctactttggattctaggtgggcattacagagttgaattcctcactacccccttcccgtgtcatttgtttattggaaactcatgttactagagnggtcccngggccggancacnctaagccgaattntggagntttcatacactggnggccgtttancattcnttanagggccaattcgccctaaangggncgtttcaaattactggccggggttacaacngnggatgggaaacccggggtcccaantaatncctggagaaatccctttncagtgggtaaacgaaaggccccnccgtcncttccaaaattgccacctnatggatngccccccttaggcataan

SEQ ID NO: 20 - DNA, 800 bp, Homo sapiens
  misc_feature (1)...(800): n = A,T,C or G
tnnttntnnccnnctcgnatccctagtaacggccgccagtgtgctggaattcggcttcgagcggccgcccgggcaggtcctctgtgataaacaatgaaaagctgtctccatgtcctgccattgacaccacaggtccagcaaggcttgnatcctctttttgacccctccaatagtaaacaatcgaagaagcagggcactagtagcggcagcagcccatccttgaccgagacatatggcttcaatatcctcattctgaggcaagtctattatccactctttgcttgaatcccaagaactaaagtgcaggcagtgaagcttgcttgctagttcatcaagtgctttcacatgccaacaaaataagcttcgtgggaaagatctgctattgtataattcaaagtgtttgataagtgtgttgcatggtgttggaggtatcatggaactcccatctatggcattgtcttgctcatcattatagcagcgaattaattccaatagagttcccaccatgaatctntgagtggagatgcaacngngttnaacctnactngaaatngntttttncccgggaggtggggattggnnccntataaaaaggcctttnggannttccaaggggnaattgggaatgctcctttttgccattttcctctccttttggaaaaactngaccnntttttnnattgaaantaaacngggttntttnanttttaggatggactttgtgtttnggccaccganncnnanangctcaaaaacnantnnttttaanccctttgaaaaaanggactccctgttnctta

SEQ ID NO: 21 - DNA, 777 bp, Homo sapiens
  misc_feature (1)...(777): n = A,T,C or G
ctagtaacggccgccagtgtgctggaattcggctttcgagcggcncgcccgggcaggtacgcggggacatttgcttctgacacaactgtgttcactagcaacctcaaaacagacaccatggtgcatctgactcctgaggagaagtctggccgttctgccctgtggggcaagggtgaacgtgggatgaaagttggtgggtgaaggcctgggcaaggctgctggtgggtctaccctttggacccagaggttctttgagtcctttggggatctgtcactcctgatgctgttatgggcaaccctaaggtgaaggctcatggcaagaaagtgctcggtgcctttagtgatggcctgctcacctggacaacctcaagggcacctttgccacactgagtgagcttgcacttgtgacaagctgcaccgtggatcctgagaacttnagctcctgggcaacgtgctgntctgtgtgctggnccatcactttggcaaagaattaacccacaatgcangcttgctattaaaaaagnggggggtngtgnngnttaatggccctggccccnaatttaactaaacttnnttttttgttngccaaatttttttnaaaggttcntttttncctaagccaactnctaanttgggggntttttnaaggccttnncatttggntttcctannaaaaaattttttttttttaaaaanannnnnnanntaaaatcctggcggacnccctagccatttganattnannaaatggggctttggaanttttangggcaatnt

SEQ ID NO: 22 - DNA, 835 bp, Homo sapiens
  misc_feature (1)...(835): n = A,T,C or G
ggccgccagtgtgctggaattcggctttcgagcggcncgcccgggcaggtacaaggtgaaatatttaaatagggaatctggaaaccaaaaacgaattcaaatctggatccaaaatcccacaattaaattggaaggtttcaatttttattcaaattggtgaataaaaatagcagaccactggtttcatccaataagcccaatggatatcaagctttanggagaaaatggatctggcctggcttggtgcaagacaagaacaggttaccttctgctgaaagggatgtgaggttttcaaatttggttttcatgtcatagtttccaaataaaatgaaccgctccaccaaaaaccgacagtctagttngctacctatcttccaggggtttcatcatacagccatttataangcacantctttggaaccgctctataggtttcaaaaaagtgttttgaaggtcttggaggggtgcatgttaactactttctcatcctcctgnttttcctatttggcatcaatatatctctcggcttcaagngggagtcaatgaaccgtaagtggggctatttnaagngggngctgnggcctcccaaaaaanggnttttaccagggggaaaaantnntttttngtccananggggtaagtcttngggaaanaaattttgccgnggaatcatttggaaaaanaannagcntttgggggnattgggccaaaaantnccccncccttgggtttccagnggcccttccgggngtnttttnaaanntgncaagggtcttngcgnaccttntttacaaggttttgccggataccttatactngncaaggtggtttcggg

SEQ ID NO: 23 - DNA, 761 bp, Homo sapiens
  misc_feature (1)...(761): n = A,T,C or G
ggccgcagtgtgctggaattcggcttagcggccgcccgggcaggtacttatgggccagggcaatggcgacagcagacaccagcttctgccaggcagcctgcacttcaggggtgaactccttgccaaagtgagtagccagaataatcaccatcacgttacccaggagcttgaagttctcaggatccacatgcagcttgtcacagtgcagctcactcagcttagcaaaggcgggcttgaggttgtccatgtttttaatagcatctccaaaggaagtcagcaccttcttgccatgggccttgaccttggggttgcccaggatggcagagggagacgacaggtttccaaagctgtcaaaaaaatctctgggtccaggggtaaacaacgaggagtctacccaaggcttaccttcagctcttncacattnatcttgcttcacagctagtgacngcagncttctcttaacagtaaaatgcaccattgatgcnngncctgaancttggttgtgatttgaantttnttnggaagcaaantttccccggttncctgggcgggacnccttaagccgnantttgnanattccataancttggnggccggtngacatgcttttaaagggccanttcncctnttgggngnggtntaaaattacnggccggggtttanactngngacngggaaacctggnttncaanttattgcttggaaaaatcccttttccagtngggtananaaaaggcccnccntcgcttcaanngtggcng

SEQ ID NO: 24 - DNA, 784 bp, Homo sapiens
  misc_feature (1)...(784): n = A,T,C or G
ctagtaacggccgccagtgtgctggaattcggcttagcgtggtcgcggcccgaggtactgtatcacttagtataccctttaaggtagcacttatccagtccaaaactccagtgacaaaattcctagtttatcaagataaacacagtnacactggattaaaggaaaaacattgctatggtatagactgtggttggcttctatccagtaaccttgggaatgaagacatctttgtaaacaagtcctgctgtttctttaacagctaacataggaaataattaaatgtattctttagtgccaattgtaagttttaaaatcaagaatggcagtgtaacttgtgaattggctagggcaatcaatcacaagcactactttctgtaaaactttagtagttcaagtgttaccagttntacccantctttggtgaattccaacttgtttgcttaagttatcttctttagnggtttcctggngggttttcaatgctcttcggnggngtcataatgccctccntngccacttggggacaactggncccccttttttgaggggntaatngaatttacttccncccacaanacccaaanagttngnatnggnctgggantntanggcngcttccaaaangaatctttttaaaaatnttnangttnntggnttaganccgcccggggggcgcttaaanccatttttgantttntnaaactggggcgtttggcttttttnagggccaantncccntgngggggttnnantaatnggngggnttaaaagtngnnnggaannt

SEQ ID NO: 25 - DNA, 782 bp, Homo sapiens
  misc_feature (1)...(782): n = A,T,C or G
ctagtaacggccgcagtgtgctggaattcggctttcgagcggccgcccgggcaggtactataacaggagttctaatagagatgagggcagagtgcccaaggatgagtagcaactgaaagcttatatgatccactgagagtagttaaaaaaaaaaaaaaatagtacccaagtctcacctcttcttcactgccggaaaagatcataagatttaaacagtgggatgcagacaattttgaccccttccagactctgggngggcttaacnggaaaatttggnngttggcttntaagccccnnngggnnctttntngnnttattcttccnnnncnaatttttngnanntttggnnnaaaccagggggccctttttnttntgaggaaaaaaggngattggaaaagaaaccanattttngnggggnnggnggccaaananaaccanccngcttttcccgggagnanctcagggggacggttcctttnntgggaaagaaactttttnaacagtnaaagcccntnggaggtttnttggnggcttttgggtngnccttggccgggnncccttnagcccganttttgtgnattttcnttaanactngggggcnnttttggcntttttttnnaggggccaattcccctttttgggggggntttnnantactngggcgttttcncccgnngntnnggaaaacccgggttncccattttttngntggggaantnccttttccngttggttttnnaaagggcccccnntncttnccaangtngcccccgnagggaagg

SEQ ID NO: 26 - DNA, 785 bp, Homo sapiens
  misc_feature (1)...(785): n = A,T,C or G
cctagtaacggccgccagtgtgctggaattcggcttagcgtggtcgcggccgaggtactgtatcacttagtataccctttaaggtagcacttatccagtccaaaactccagtgacaaaattcctagtttatcaagataaacacagtaacactggattaaaggaaaaacattgctatggtatagactgtggttggcttctatccagtaaccttgggaatgaagacatctttgtaaacaagtcctgctgtttctttaacagctaacataggaaataattaaatgtattctttagtgccaattgtaagttttaaaatcagaatggcagtgtaacttgtgaattggctagggcaatcaatcacagcactactttctgtaaaactttagtagttcagtgataccagttctacccaatcttggtgaattccaacttgtttgcttagttatcttctttagtggtttcctggggggtttttcaaggctcttcggnggggtcataatgcctccnttggaanctgntgacaactgnccccttttttgaaggggttattgnaattaacttcctccacataccaaaaagttggtatgngtcggganatatgnctngcttccataggaatctttttaaaaatctttaacttcactgggntaaganccggcccgggcggccgttgaagcccaattntgnaatnttcatanactggnggccgttgggcatgctttagagggccaattcncctttggggnngtttanaatacggccggngtttacacggnggacngggaat

SEQ ID NO: 27 - DNA, 777 bp, Homo sapiens
  misc_feature (1)...(777): n = A,T,C or G
cgagctcggatccactagtaacggccgccagtgtgctggaattcggcttagcgtggtcgcggccgaggtacctgacgcggggcaagattctgctgtgcacaggtgccatcatggaagaacaggcagcagaactccttggagtgaagatgtgcacgtttgttccaagacacacccggaacttggcaaatgagtttcgctgttatgtgaattatgattctgggctggactgtgggatctgattacagacggtgacataacacaggaaggaaccctgtcacattcctctttttgtattttcgtagtagatttaaaagttataatctcttcccctccccccaaactggagctgtccctggcctggttttcaaaataaagtgtgcgatctccananaaaaaaanataaanantaaaaanggtncctgcccgggcggncgctcgaaagccgaattctgcagatattcatncacttggcgggcgnttcgacatcattttaangggccaattcgcctntagggggcggattacaaattaatggccgcggtttccaacgtngggactgggaaaaccctggggttnccaactttantcgccttgagnaaatccccntttgcagttggngtaatagcnaaaggccccnccgatcgccnttcaacantgcncacttgatgggnaatggccccccttacgggccttaaacccgggggtnnnggttnccccaggtgccgttcanttgcanggcctaggcccgtcttggttttccttnttttgccgtngngg

SEQ ID NO: 28 - DNA, 787 bp, Homo sapiens
  misc_feature (1)...(787): n = A,T,C or G
ggccgccagtgtgctggaattcggctttcgagcggccgcccgggcaggtacgcggggagagcagagtacgagtctgaggcggagggagtaatggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagccgtgaaagttggagataaagttcttctcccagaatatggaggcaccaaagtagttctagatgacaaggattatttcctatttagagatggtgacattcttggaaagtaccgtagactgaaataagtcactattgaaatggcatcaacatgatgctgccattccactgaagttctgaaatctttccgcatgtaaataatttccatatttcttcttttttaataaactaatggttacntaattgccntcccanngntctccaaaattnntttccttggcccggatttaaacnccttccaaataaaaatttgggaaannaaaaaanntnntntnnantntngggtcccttggncgggccccnctaagccgaattntggaanttccttacactggnggccgtttgncttgcttttgngggcccatcncctttagggngnngtttacaatnactggccgggtttcacgtngggctggaaacccngggtnccaacttattgcttggaannt

SEQ ID NO: 29 - DNA, 801 bp, Homo sapiens
  misc_feature (1)...(801): n = A,T,C or G
tnntnnnactnggtccctagtaacggccgccagtgtgctggaattcggctttcgagcggccgcccgggcaggtacatttgggaacacagagaaatcatcttaggtatataaaagaaattcaattatgaaaagccttgaaaacgaacctgaggagtttggactaaaagcaaaaagaaaccattataagaatttatgaagtagagtgacatcatgaaagttgtcttttaggaaaagtcacctggcaacagtatgaaaggtgaattaggagagaatgagaggatgtagattagtcaagagactatcagttattggatgtgaattaaagagatgacctttgtaatagggaagagtggagagactgaggcaatttcagaaaactaggtagagccattttctcattaagagcctttggctgcaagtaacagaaatgagattaaacaacaagaaaaaatattgagtcccactgctggttatccaaatgtagtgccctttcangggcangctttcctccagttttttttccacttcattttttccaggattnaattggcctcaagggctcntttnttttgcnaggcntttttttccagggtggcttgaaccctagggactttanttccatcacntccncccatgaacaaatccatgantggttgtccttggccgggaccnccttaaccgaattttggaatttccttacacttggggccgtngncatcttttaagggccattcgccttaggngngtttacaataantggccgggtttanacnngggcnggaaacccngntnccaattntc

SEQ ID NO: 30 - DNA, 802 bp, Homo sapiens
  misc_feature (1)...(802): n = A,T,C or G
tcntactcgntccctagtaacggccgccagtgtgctggaattcggcttagcgtggtcgcggccgaggtacccaatgaaggatccgatgaatggctggccagttactcaaagtaatatgtttatgagttaaatgggattgcataacggcaccacttttcctttgtcactgtggtggataaatgacaaagtagattccaatctcctgtatagaaaggtggttacccaatagcctcactggtggcttgctagcagagcctcaagttgcacctctgtggctttctgtaaattcgagtcctacagccacttcgagaacgctgcaatccccaaggcccacttggtccatgctgaagaagcggctgtgaccccaatcctgcctctcttttgctcttcagtattacttactggaggaatatgtnatattctaggtcaagtctangagaggctcatcctaaaaaaaaaattgcaaaagtcttttctnnattaaaacaaaaaaaagcctcaagtncctgnccnggccggccgctntnanccgattnttgcananttcatttaanattgggggccggttgngcattgcttttaaaggggcccattcncccttanggnggcggnttacaattcaactggccgnngtttacaacgtcggggactgggnaaaccctngnnttnccaacttattngcttgnaggnnattcccctttcgccagttggngtnatacgaaaaggccccnccgatcgncttncaaanttgccacctnattggnnatggccccccttatngncntancgcgggggnggngnn

SEQ ID NO: 31 - DNA, 751 bp, Homo sapiens
  misc_feature (1)...(751): n = A,T,C or G
ctagtaacggccgccagtgtgctggaattcggcttagcgtggtcgcggccgaggtaccactgtgattttgctaaactttccaaacttacgctatcatgaagttttttccatttaatttgagtattgctataaccagttttgattcaaacaaaatacaaaccaagactgtatcagtcccacagagagaaaagaatactgaatgacttatagggttaatgaaatgttctacatagccatttaggccagtttctgaaatggcactacagctaacttgcttagagttttacctgggctgctggaggaaagctatggcacgaaagccgatgtctttcctataaattgctccctcaaactttatagcagctagtcctttcttggcttcctcaagggctgatatgagattttcccggatggctgtgactgcaagaactctacccccatgagttactactttgccatttttgagggcagtgcctgcatggacacctccagcctagagcttgagcctaaggaacccttgtntttttccccccttggggnancntcaggntaacctttattggccatgacaccagttangggngggnnggttttntaccaaccngcagantgggcanacagccatttaaggggncngatcacttataagaanctttanaagggggaggatactggccttggacacaaacggaataacttaaacttnggcatntggcacatntcactaaaaaccgnttgggnccgccgggcc

SEQ ID NO: 32 - DNA, 787 bp, Homo sapiens
  misc_feature (1)...(787): n = A,T,C or G
tnnntcctnctcggtccctagtaacggccgccagtgtgctggaattcggcttagcgtggtcgcggccgaggtactctgttgtatagctaatacaagttaggatgcttttggccagaggtaacagtgtccaaatataattggcctaagtaacctaggaaattgtttgacataacacagggttcaggggtgtcattaaagacacactttttttgccttgacctcagttggtttgttttgccttaggctattccacctctcgatcacaagagctgctgctataacttccagttttacatctttgtaaaattgtatctcaaaggcagaaaaggccatttcgtctctcatttgttttatccatgaggaagatttttaacaaaagcctccagangatttcccctcagtttccattgacttagatcaggttacagagaaaggcaatgtctgacattttttggtctctgttagaagcagactctgttgaaaagaaagaagctaagctaggtgtgaagaatgggatttggaagcccnctngctttccattagaaanggttncntaattaccccctttttttggggtaggaatttggatttttggangttttnncnccaaccannggcaaagggattgcnttgncctnccggggggncgttnggntgctttttanngggccaattcncctntangggggtttacaataactggccgnggtttacaacgtgggctnggaaaccctggngttnccaattattgcttnngaanccctttggaggtggttanacnaaggcc

SEQ ID NO: 33 - DNA, 773 bp, Homo sapiens
  misc_feature (1)...(773): n = A,T,C or G
ctagtaacggccgcagtgtgctggaattcggcttagcgtggtcgcggcccgaggtactctggagtcaccagcctctggtagattatcaagcatctcttcctcatcagccttttgtttctgtcttgcatgcttctctgtccactgtctggcattcttgaggaaggctggcttattatatttaaattctgaggatatgtcagccatgagcgggtcatcagggttgggttctgacatgagcagctgaatagaggtcaacacagttgcgatgttgagggatggtctccaagcaccttttggtggcaatttgagaacatccagacaaatccttccagcagaatcaatgtttggatgataaattggagtgagaaatcggatctgaggaggttcaaatgggtacctgcccgggcggccgctcgaaagccgaattctgcagatatccatcacactggcggccgctcgagcatgcatntanaggggccaattcgcctatagtggagtcgtattacaattcacttggccgncggtttacaaacgtngnggattgggaaaaccntgggttncccaacttattcgcttggangaaatcccctttgccagtggngttttagcnaaaaggccgcnccgatcgccttccaanngttgcncacctgaatggnaaatggcccccccgtacggccatttaancgcgggggtntgnngttncnccaaggtgacnntcattgccangncctaggccgtctttgtttttcntctttttgcaattncngnttccc

SEQ ID NO: 34 - DNA, 776 bp, Homo sapiens
  misc_feature (1)...(776): n = A,T,C or G
ctagtaacggccgccagtgtgctggaattcggcttagcgtggtcgcggccgaggtacacatttgctgatggcttctcaaaacctgagccgagaatagggtctgatagcccagccaagtttaaaagcagacacacacgaatgtagtatcgttgtgcctgaaatgaccattctgggttgtttagaatccagaatcatcaaaagccatgtggtatgaggaagtaataaatatcctcttgaatcttcttaccctattttgcacaaatggatggctgcatgaacagctcttgtaaattgctctgagtccacaccaatagaaacctgcactcattctatagctacagagggtttgttggcttaaggggactttatcatctcagcattaatttcccttttaaagctattctcaaggntggactgtctcagagataaacaaagaggaatccttttggcttagaagccaactggcttactcagactttcttccctttctacctcnatttccacacttccaatattatctttttggactaggaaatcaaatnttttcntggactaanggggcaggttattttttttccaaccctgtccctggnggccaatgggaagaaaatncccggccctttgggaanngnatttgancngccttttgttttaaccnttangcccaanctccanggcnanttngaaggccntaagggttnactgcnggaagccactcccntgnaaagtgccatcntntacatgggntcaacgggactttcaggtttntttcttgg

SEQ ID NO: 35 - DNA, 772 bp, Homo sapiens
  misc_feature (1)...(772): n = A,T,C or G
ctagtaacggccgccagtgtgctggaattcggcttagcgtggtcgcggccgaggtaccaacagtgataataaattctccttctggatcccagactaggtcttggacaccatcaaagtgtcctgaaatgacaatctctggagtccactctctctgttctagccaaactcctgactcttcatctggagcccagagaatcatggttttatccatggaagcagataataatctcattggctgctgtaggacaccatctttgtaaaacacaggttgccagtgaactgcatttacccagttttcatgaccggctagcactgtctccagagtaacagcaaatgctattttaacactttcattttctatggtaaaagtattttctttcagtcttatgttatcgtcatcctgagtttctaaagatgttgactttatatacagcttccatattcttatcaggcaatcttgtgaacagcttgctaggaaaaagatctctacaaangntggccatttcactcctttaatccaatccctatgttcacagagaggnaagaccttntggaacctgtnaatttngtggngcaaantttgaatttgnaanattattgccnatngctaannttgtacctgcccggnggccgttcnaaagccgaattttggaaattccttaanactgggggcgtttgagnttcattaanggccaatttgcctnaggnggggttcaattaanggcgngtttaaacgnggnnnggaaacctggggtnccanataatgnntgaa

SEQ ID NO: 36 - DNA, 774 bp, Homo sapiens
  misc_feature (1)...(774): n = A,T,C or G
ctagtaacggccgccagtgtgctggaattcggcttagcgtggtcgcggccgaggtacatgacctaatttttacatcatagtaaaacaggccctatggagagaggacatgggtttctctgctgaacagccattatttatactcgttccaaggcttctaacatgatgatactatttcctcgtattaccaccattccaatattgttctgttgtccactagtcgccatctccacacattcatctatcacaaggttcataaagggatcaaatccccgcaatattccttggacatgtctgccaccatttaatttcaatgataacttcttgtccataaattttttcaactcgggagggtgagctttgctcatggtgtatactccgcgggctcacagatgccttggaacgcaacgcacgggtttcctnacgcttccgctcccgcgtacctgccgggcgggcgctcgaaagcccgaatttgcagatattcattacactggcgggcgttcgagctgcatttttanggcccanttcnccctttnngggngtggnnttacaattaactngccggnnttttaaacgtcgggactgggaaaaccctggngtncccaacttnatggcttggagcnatncccctttngncagttggggtaataacnaaaaggcccgaccgntcgcccttccaaangttgccaacctaatggnaatggacccccctgtacgggcattnacgcggggggggnnnttcnccagggnacnntaaattgcagngcctagncgttt

SEQ ID NO: 37 - DNA, 818 bp, Homo sapiens
  misc_feature (1)...(818): n = A,T,C or G
ttnnnnnnnnncnnanctcggatccctagtaacggccgccagtgtgctggaattcggcttagcgtggtcgcggccgaggtacttaaatcagaaggatcgtaggcctgataagcaggtcaacctgaagctaccaggcagctctggaggcttccgtggccatagctaacagtgcacaggagggcccctggcccaggatgagccctcagaaccttttagagagagtgaaattcagggtctactgggtgtccaagctgcagagtggagccgtgagaggtcagctgccatgtgatagaaagagctgacataagagaaaagacccccacacagtgaaaagctggaagagaaacaaaactcacaacctgacagcagagttcctggttccaactgttgagacctgtctctacctccaatggtcacaagtatatagttacccaggcctggaaatctccctcttttcctaattagtttaactnggtgttcctgntngttggaaaacagcaacctgnattttttaagacacncttgngcaaagttnaaaaggnggtggggaccggtcctgnaaagganatgatgggnaaggcaaaacttggctggcncnantgtnttttttggccaaaaaggnaagggggggccctttttttntngcaaaaaccctttttttttggaaataacttgggcntctttctngnacctgcccggggggcggtnaagccnattttggaattcnnaaacttgggggcgtttnctncntttaagggccattcccnttnnggggggtttnaantaatgggcgcggttaancggggangggaaanccg

SEQ ID NO: 38 - DNA, 815 bp, Homo sapiens
  misc_feature (1)...(815): n = A,T,C or G
agnnagnnnnnagnagagnggnnnnnnnttnnnnnnnnnnngnnccgagctcggatccctagtaacggccgccagtgtgctggaattcggcttagcgtggtcgcggccgaggtacaatatatttactaggggaaaaactgtctatagtagcaccatcttcttcctttgaatgggaaaagtttgaccaagaagcaacataaagtattagcagcaaaatactgttgaaccagctatcatccccaagactcccactataaacatgttagcaacagtgatacacactgccttacagcaatttttggctgtaaaaatctagccaattcagttttttgatatttggtttttaatagtcttgttcaagtaagaaactagctgctagttggactgagcctgttctgaaaagacatggaagattcattagaggcacacaattgcttaatgtgagacttactgaaaaacaaaagtcacattaccccctagcttaaggcttcatttcctaaaaagctgacgtgatttatttttagaaccacctgtcaagtcaagtcttggaanggggattctgatttgaagtatttctaacctgatgttagacccttaaaaactcaagaccatcttgagtnttaaccacccttaattaatcattttttatagaagcccaaaacancccaattttttttcctttttttgaataaacaaaagccctttgattccatttgccgacnctttttaaaaaattcccttaaaattcncttggaaanttttnatnagnggacaccgnntgtnttccttganggggnanntttgcttattcnttt

SEQ ID NO: 39 - DNA, 816 bp, Homo sapiens
  misc_feature (1)...(816): n = A,T,C or G
agnnnntnnnnnnnnnnntnntnnnnnnngntgntccgagctcggatccctagtaacggccgccagtgtgctggaattcggcttagcgtggtcgcggccgaggtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacctgcccgggcggtcgtcgaaagccgaattctgcagatatccatcacactggcgggcgctcgagcatgcattntagagggcccaattcccctatagtgagtcgtattacaattcactgggccgtcgtttacaaccgtcgtgactngggaaaacccttggggtncccaaacttaatngctttgnagganaatcccctttttncantngggtaanaccnaaaaggccncncnatcccccttnccaacattggncaacctgatngngantggacccccctgngcggnccataaaccggggggngnggnggtaccccacgtgacnnn

SEQ ID NO: 40 - DNA, 821 bp, Homo sapiens
  misc_feature (1)...(821): n = A,T,C or G
nnnnnagnnnnnnnnnnnnnnnttntnnnnntnngttngnaccgnctngtccctantaacggccgccagtgtgctggaattcggctttcgagcggccgcccgggcaggtacgcgggacgggaagtgacgagagccgggtggacccagggtggggaactacctcttcctctccacgcggttgagaagaccggtcggcctgggcaacctgcgctgaagatgccgggaaaactccgtanngacgctggtttggaatnagacaccgnantgaaaaaaggggagacactgcgaaagcaaaccgaggagaaagagaaaanagagaagccanaatctgatnngactgnagagatagcagaagaggaagaaactgttttccccaaagctaaacaagttaaaaagaaagcagagccttctgaagntgacatgaattctcctaaatccaaaaaggtaaaaaagaaagaggagccatctcaaaatgacatttctcctaaaaccaaaagtttgagaaagaaaaaggagcccattgaaaagaaagtggtttcttntaaaaccaaaaaagtgacaaaaaatgangagccttctgaggaagaaatagatgcttcctaagcccacgatgnttngcttncttaccccanaaccaantnggtcccttggccgggacccccttagccgaattntgganatttccatnanactgnnggcggttcgncttgnntttaaggggcccattccccctttagnggggnngtttacaattactngccggcgnttnnacgtngtgncgggaaacccggggtacccacttatngcttgagacatccctt

SEQ ID NO: 41 - DNA, 825 bp, Homo sapiens
  misc_feature (1)...(825): n = A,T,C or G
tagntannnnnnnnnnnnnnnttnnncnntnnnttnnaccganctcggatccctagtaacggccgccagtgtgctggaattcggcttagcgtggtcgcggccgaggtactgcccacgaagttcttccaaggctcccccactgcatggcaggcaagcattttcaatggcccctgctgacaatgaccttggtggtgttttacaaagtgtttgcaattcttccaatttattcttcatgtcgttgttttcttctattaattcttcaacaactttattattctcttctccaattttttccactacagctttgtgcttcctttctaaatgctgaagatgcctacagcatttctccagttttcctttcagatcttgacattcctgctttgccttttcaagttcttggaagcagtgtccacagcataaatccttaatttcaaggacctttttcttatcagcttctgcttctgttgcttgtaaggccaagggctgagcatggtgtccagcagcaggtccagggtctccttggccagtgccctggctggcagaatccttagagagctcggccatatttctctttttccattctgggtcaacggttttctacaagcaaangaagtgtatcttggaaatgctttctttatgcagccctttagtttttcaaaaanattgcacaagaccatccatttttgaaaaacaaggcgacattgttttccaaaaagcttgnttgtcctaaaancnactganngctcttttgggttcactaaaaaaaacccaagncctgnccgggngccgttcgaaagccaattttcaaaatcatcnnccggggcg

SEQ ID NO: 42 - DNA, 799 bp, Homo sapiens
  misc_feature (1)...(799): n = A,T,C or G
nagnntnnnnnnnnntnnnnnttnnnnnccngttngnaccggctcggatccactagtaacggccgccagtgtgctggaattcggcttagcgtggtcgcggccgaggtacgtctccactctgacagatcaggtgaccctcctcatcatcctctacactcctggttcttttccttcggtgactcttctggaaacgtcaagtgggcggcaccaagatcatccagccaatcaatatacccgagtgaattcagggcagaatacgaaattcattcagcggggagatattcaggcattcagatacacgccaggccacaaacggttggcaggagcaatttcaaattcagtccccagaaattcacagggcacatagtttatgttaacaggagaaaaacatggcaaggaacagattttcagataagatactcaaagtggcaaggcaacaaaacataatacaattgtttttcttaaaaaaaaaatgcttagttgtagcattcactgaaacataattttttagtttctagtgttctaatttaatgttgcaaaatcgtatgatgtaatatttacttgtcaaagtaggcnggnggccaaattaaagattcctacctatctaagaaaacaaatnttttaccgtaaataaattaaagngctaattttggggaaaaagattngcntccaaaataccctacgggattttaangcatctgactgnctcctntatatntanccctacagtcaaattttntggttgctagnaaaaaggttaaaanatgnttgtanacaaanagcttcctatctaatttnc

SEQ ID NO: 43 - DNA, 806 bp, Homo sapiens
  misc_feature (1)...(806): n = A,T,C or G
nagnnagtnnnnnnttnnnnnttntnnnnnnttngtnccngctcggatccctagtaacggccgccagtgtgctggaattcggctttcgagcggccgcccgggcaggtaccccaacctccagcatatcaaggggtaacagttccaacaccaggtcaggatcaagctcagcatccaatgtcacccagcgttatagctcaacctttggacctgggacttaccatcactccagaacccactacagaggttgaacattctacacccctgaagaagattcctcccaagcaccctaaagtgacacttcacatccagaccaggttcagactctccattcgaacctgactcaagtcacagttcaacctttggatctggaacttaccttaactccagaatccactatggaggttgaaccttttccaaccatgcagaagaccccaactcagcctccagagctacgtaaggaggttgtagctcaacctcctgtgtattatgagacgtncatgccaacacgaggccaggatcaagctcagcatccaacatcacccagagtcacagttcaacctttggatctggggcttaccatcacttccagaatccattacaaaaggttnacccggntacagccttgttgattcaggttcttctcaaaggaccttgngngggnncttcccccnccngacaaggntaagggtcaaaatttnaaacctnatttaagntacagttaaactttggacnggggcttccttacttttgaacntctttgtgttaaccgttcacccgggggaactaactntttcanctggntgcnn

SEQ ID NO: 44 - DNA, 795 bp, Homo sapiens
  misc_feature (1)...(795): n = A,T,C or G
agnangnnntnnntnnnnnnntnnnnnnnnnntnnntccnnctcggatccactagtaacggccgccagtgtgctggaattcggctttcgagcggccgcccgggcaggtacaaaattaacatcaaaagacctttgtaaaatgatgccttttgatgatcttgtgacaagggtcaggagatgcggagaagggaaagggaattggtctggcaggctgaggaatgtgctccacatgcaagtgaaaaagcgaggatctgctaagctcccggaaactctgcctcttgattagtcatcctgggacattacccaaccttccttgtgtctgttgctgctgagtttcataatcccattcaatctacactataatcccattcaattttacaatccattataattctgagtatgaaacagatatttggggagtgcgaaaaggagaagggaaaaagcaagcaaagaaagtgtcttcagaccaagcatgcagtcattgagaggaagaatctggagttcgtcaggttgaccatattcataccctgccaaccaagctcaaaagaatcttatctctattggtttncagagaattgtctgtgagagacaacgttgaagctctgctgattaatgnttggngaacaacccaaatttttttcccttngncnggnncncncttagcngattttganatttcatnncctggcggncgttgacattcatttaangggccaattcccttagngnncgtntacaattnctgccgntttnaacgtcggantggaaacttggtnccactantgcttgacaatcc

SEQ ID NO: 45 - DNA, 814 bp, Homo sapiens
  misc_feature (1)...(814): n = A,T,C or G
agnnagnnntnnnnnnnnnnnttntttnccnncttnttccgagctcggatccctagtaacggccgccagtgtgctggaattcggctttcgagcggccgcccgggcaggtactgagccacgatttgttctctcaacacacaggtgtttcttttctctgactcaggattctgagaagctgctgcactggggaccattcttttctcaacctcaacctgaaagtatctgccttctaattcatcaactggctgggtgctttctaaaatggtgtcattgttttcaatgagtttccaatcatcattctccagaaaggaaagaaatctgccatccttgcacagtgcatccttgatggtttctcctttgaaagcataaacacagagtttgcgcccctttttgtgaagcttcccacatttaacaatccttcttttacacttcccaattccaattgcatgaatgtaaaatttgacacattcagtcgatgctttgtcctgcctgccaaatatgtggttatcttccttctgcttacttttactttgggaaaatgtnatgaccacctggccaccttcanggaaacactgaggggcattncaaggttttgtacctcggncgcgancacnctnagccgaattctgcaaantttcatcacaatggcgggcgnttnggcattgcntntaaanggcccaattcgccttttntnantcnnattaacaattnannggcgncgtttaacaacntcnggantgggaaaaccctgngntaccaactatntncttggananatnccctttntccagtngnntaaacaaaagccccnccg

SEQ ID NO: 46 - DNA, 815 bp, Homo sapiens
  misc_feature (1)...(815): n = A,T,C or G
agntnagnnnanntnnnntnnntnnnntnnntttcttnnttttntnnnnantnancctttnacggncgccagtgtgctggaattcggctttcgagcggccgcccgggcaggtaccgggggttaggacttcaacttatattttgggggagggaaccacaattaaatccatgggagccagaaaatgttttcatttgccatttaaggaaacttctgtaaggaaaaaccccatatccctgttgcaggtgtgatggtgaagcaggtgtgagagctgagcaggcaggagttgcacaagtgattcccttgtctattacttgagtggccagacaccaggaggcagcaagtagatttctccaccgcctctgactantgaagccctgggagttgcttaagcttatctactaaccaccttgacaggcttgtaaaaattttgcaacatcattcttaaaaaagaaagaaaatccagcagccagcaaactgtccataacagagccaaatgactcactgccagcactgctctacatgttttaggaagtccttatcatctttccgccaacacatccaattatttttaaagcagcttttttccagagggatgcgtttatttttttgnngtaatggcattctattccaaaagttaaccccgntggttnccaatnggagnttctccagaggcataatnggccagtgtttaaaaacagcnctccaaaaaaaaanctnggngggggggaagggctgnatagtctttgcaatttccntggggtaaccccccnnatggttnattcaacctctatnggangtcctngcnggacccctat

SEQ ID NO: 47 - DNA, 809 bp, Homo sapiens
  misc_feature (1)...(809): n = A,T,C or G
aagnnnntgnnnnnnnnnnnnnnnttnttnntnnnntnnnncngagctcgnatccactagtaacggccgccagtgtgctggaattcggcttagcgtggtcgcggccgaggtacctcatacacacagttctgtgagatacctaggtttgggggccaaattggaatagataaaattctttgtccccgtgaagactgttcctggccagatacctttcctatgggaagtccttcataattcaattttccttcctttatgaaactatatagtggagaaccaggaacagaattaaagtcaccacacataacaatagggcagaagctgccatctttctggtgggcaacactggaaatctctgccagtagcattgccaattgcgtcagcttaatatcacctcgccttggattatacaacagatgcgtatttgctacgcagattgcagggcaggcagcatatggaattttgggctgtaagagtaaaactaatccaacattgtctctgtccaacagagaaatatcagggcggaagaattccactgggttcactgacaagagtgaaaatttggaatgtttgaagcaaatagcacaagccatcaggttccttctggccgcattttatttcacagggntaacccagtgattccaacttggncngantttggtcattaangaacttttngacttttggnaacaaagacctgccgggcggccgttcgaagccgaattntgcagattncntanactgnggccgttgagcttcatttanaggcccaatcgcctataggngcngatacaattactggccgcggtnnt

SEQ ID NO: 48 - DNA, 833 bp, Homo sapiens
  misc_feature (1)...(833): n = A,T,C or G
nagnnnnnngnnnagntnnntntnnnnnntttttttgtngnttntnccggctcggatccactagtaacggccgccagtgtgctggaattcggcttagcgtggtcgcggccgaggtacagggagaactcacagctccaagagcttccctccaaattatgatgaagttcctgtaaataactagatttcctacagcctggtcctctgcccatgggaaggaggggaaatggatgtgaaaagcaatgtggtctgccctcctgtcattcatagatccagttctgagcacagctcgtgtaatctaccagttcctcagggtgaaaccacaagccgatctcnttctctgcactctccacagaatcactgccatgtataatgttcctgccaacttgtatgcagaagtctccacggatggtcccaggctttggagtctncagggttggtcttccccgagcattgacttggcccgtctttaccacattcaagcccttccagaccatggncaactacccggcccttgagtgcatggtntttcaccaaggccggcaaagaaatggaccggnnccttcangncaacgtaagtggttcctttgagaagancnttcggaagcttggcatgaatttcanaccaacaaggnggnatcctttttggtnnaaacgcttggtanttttttcccaagaccccggttggacccatttggtttgatccaattaaaggtccntgcccgggnggccgtttaaagccaattttggaantttcttcacctgggggcgttgagntgcnttaanggccaattcnccttngggggngtttanaattactggcggggttaaacgcg

SEQ ID NO: 49 - DNA, 807 bp, Homo sapiens
  misc_feature (1)...(807): n = A,T,C or G
nnaggnaggtnnnnnnnnnnnntnntntnnnntnnnttnntnnggcnnggatncactagtaacggccgccagtgtgctggaattcggctttcgagcggccgcccgggcaggtacttatgggccagggcaatggcgacagcagacaccagcttctgccaggcagcctgcacttcaggggtgaactccttgccaaagtgagtagccagaataatcaccatcacgttacccaggagcttgaagttctcaggatccacatgcagcttgtcacagtgcagctcactcagcttagcaaaggcgggcttgaggttgtccatgtttttaatagcatctccaaaggaagtcagcaccttcttgccatgggccttgaccttggggttgcccaggatggcagagggagacgacaggtttccaaagctgtcaaaaaaatctctgggtccaggggtaaacaacgaggagtctacccaaggcttcacctccagcctcttcacattcatcttgctccacaggctagtgacggcagccttctcctcagcagtaaaatgcaccatgatgccaggcctgagagcttgctngtgattgcagctgtgtcggaagcagatatgttcccgcgtaccttnggcgcgaacacgctaagccgaattntgngatttccttaaanttggngggcggttcgngcatgcatttaaggggnccaatcncctntatgngtnggttanaattnatngccgnntttaaacgnnggnctgggaaacctgngttnccacttanttcttganaatncccttttgccngtgggannacaaaagg

SEQ ID NO: 50 - DNA, 827 bp, Homo sapiens
  misc_feature (1)...(827): n = A,T,C or G
ngnnanngnnannnnnntnnnnnnnnntttctgtnccattntggnnccgagctcggatccactngtaacggccgccagtgtgctggaattcggcttcgagcggccgcccgggcaggtactttttttttttttttttttttttttttncgttacatggttttatatttattttattgaaattgaacatctnttttacaaatgctaataaatgaatctattcattctctcatttattcaaccaatgttaattgagcacttactattacatgccacttaaccagtttcactcagtttaatgaagcatttntataaatccttgagcacagacgtttctatgtatattgcaagctgtgtttgacaattgagtagcaattgctgatgctggaaaaaaactaatgttgcaaatatttnttgagtaattatatcagttgttttggggtttggcttaggatgtgggttcagaaagtctgaacctttcctggttcttttaatcttcaaagaaaaatngggccctgctgctgagttgananaaaataatcctnggncccccagcaaaccnnaattcnccccccaacatngggntaaaaaanttgtagcccgtaccttggnncgngaccccctaagccgaattttgnagatattcantaacctngngggccgtttgagcatnctttaangggcccantcnccctnannggggggtgnttcantanctngccggggtttcnaacgnttgnntggaaaaccccggggttnccaanttatatgnntnggngnnanccctttgcangtngggaatannaaaggncccncantggctttncnnt

SEQ ID NO: 51 - DNA, 830 bp, Homo sapiens
  misc_feature (1)...(830): n = A,T,C or G
agtnnanagnagtnnnnattttnnnttntttattntnntttntnnngagctcggaccactanttacggccgccagtgtgctggaattcggcttagcgtggtcgcggccgaggtacagggagaactcacagctccaagagcttccctccaaattatgatgaagttcctgtaaataactagatttcctacagcctggtcctctgcccatgggaaggaggggaaatggatgtgaaaagcaatgtggtctgccctcctgtcattcatagatccagttctgagcacagctcgtgtaatctaccagttcctcagggtgaaaccacaagccgatctccttctctgcactctccacagaatcactgccatgtataatgttcctgccaacttgtatgcagaagtctccacggatggtcccaggcttggagtctgcagggttggtctccccgagcatgactcggcccgtctttaccacattnangcccctcccagaccatggcaactaccggccctgagtgcatgtatttnaccaggccggcaaagaatggacggtccttcaangtcnacgtagtgttcctttgagaagatctttggaangcttgcattgatttcagaccaacaaggggggaatcctttttgttaaaaacgcttggntaattttttccacaaaaacccgttggaccccanttggtttggntcgcattgaaagntccntgcccggcnggncgntntaaagccgaattttgcaaattnatcaanctngngggccgttggncttcnttaanggcccaaatcccttanggggggtttcanatacnngcnggtttaaactnngnn

SEQ ID NO: 52 - DNA, 826 bp, Homo sapiens
  misc_feature (1)...(826): n = A,T,C or G
tagagttnaggnnnnnantnnnntntntntttnannntttngtaccgactcggatccctagtaacggccgccagtgtgctggaattcggctttcgagcggccgcccgggcaggtacaagtgcaaaaaaaattccatcatttgtaaagagaaataaaaagtgccagcctgcttgttcttaaaaatgcctcccgacataattgttcaaagacaaattagacagatgcaacattaaaaaaaaaaaacaaaaccttcttagcatgccatgtctaataaacacatatatacacaaaaactagaacaaaatcatgtgaaaccgagttatcaagctggcattgatgaactggtctgtaagttgttgggttacatcataaaatgggatgtttcacatctcgggcacagacgacttggaggttgctctcagagggcagactttttgtanaggaagtccggcttgcttggtgacctcccttccttctgcttattgaatcttccctttccaaatntgtntttctttgangctccttggcccagggactcactgtttgcagtcttctaccggattttttttngggagcttnagagcttcnttccaaaaagtgaaggtncngtntggagtccagcccaaaaagtnanggcagtttttactgnagttcggtccctnncttgggttggaccggtaccttgccgngaccccctangccganttttgnanaattcnnanctgggggccgttngnctncntttaagggcccattcccctttaggngggnntananttannggcggnnttnnacgtgngantgggaaacctgngtcccaattaan

SEQ ID NO: 53 - DNA, 752 bp, Homo sapiens
  misc_feature (1)...(752): n = A,T,C or G
cctagtaacggccgccagtgtgctggaattcggcttagcgtggtcgcggccgaggtactttaaatttagtggttgctggagcacctaaaagtcagattgtcatgttggaagcctctgcagagaacattttacagtaggacttttgccatgctatcaaagtgggagtgaaatatacccaacaaataattcagggcattcaacagttggtaaaagaaactggtgttaccaagaggacacctcagaagttatttaccccttcgccagagattgtgaaatatactcataaacttgctatggagagactctatgcagtttttacagattacgagcatgacaaagtttccagagatgaagctgttaacaaaataagattagatacggaggaacaactaaaagaaaaatttccagaagccgatccatatgaaataatagaatccttcaatgttgttgcaaaggaagtttttagaagtattggttttaatgaatacaaaaggtgccgatggcgggatttggcttcacttaggaatggtaagttgngangnagatatgttttnaaanccttcatggntcagctttnttttaaaagagggcaaacacaggtgctttntncctgccnggcggccgttcgaaagccgaattttganantttcatacacttgnggccgtngngctgcatttaangggccaattgcntttagggntngnttcaattaatggcggggttttcaacnnnnngctgggaacccgngttt

SEQ ID NO: 54 - DNA, 821 bp, Homo sapiens
  misc_feature (1)...(821): n = A,T,C or G
annnnnnannnannagnnntntnntttttnnnntnttgnggnccganctcggatccactagtaacggccgccagtgtgctggaattcggctttcgagcggccgcccgggcaggtacggagctgagaggaagatctccaggcacagcgttttgggtgcagctgtcancgttggagttccacanggcgtttctctcanagtcaagctcaacagggccagtcagacatacttcttccctattcacttgacggaccagcttctgccancgccatgttctatgccacccgtggggcctctagtaggtctactttgccatgcaccgtctgatcatcaaaccatacctcaaggctcagaaagagaaggaattgggagaagcagagggaaagcgccgccacccnatgtgctncagaagaagcaagaggcggagtccgcttgtcccggctgatgcatgaatctgtcccgaaggataattgaggcanaagagtccagaatggacctcatcatcgtcaatgcctggtacctcnggccgcgaccaccgctaagcccgaattctgcagatttccattacactgncngnccgntcgagcatgcatttagaggggcccaattcgcctntanntgaggcgnattacaattcactnggcgggggtttacaacgtctggctggnaaaaccctggnnttncccaactnattgccttgnanaacattcccntttgcnagttngggtaatattanaaaggccggaccgatngcctttccaanaatgcccacctgattggnnatggccccccctttccggccaatananccggggntgngngn

SEQ ID NO: 55 - DNA, 733 bp, Homo sapiens
  misc_feature (1)...(733): n = A,T,C or G
cnnnnnnacngncgncagtgtgctggaattcggcttcctagcaagccagaggaagggcttttcaaagttgtagttacttttggcagaaatgtcgtagtactgaagattcttctttcggtggaagacaatggatttcgccttcactttcctgtccttaatatccactttgttgccacacaacacaatggggatgttttcacacactcgtaccagatctctatgccagttaggcacattcttgtaagtaactctcgatgttacatcaaacattatgatggcacactgggcttggatataatagccatctctcagtccaccgaatttctcctggccggctgtgtcccatacattgaacttaataggtcctctgttggtgtggaacactaggggatgaacctcaacacccaaggtggctacatacttcttctcaaattcaccagtcaaatgacgtttcacgaaggtcgttttttcagtaccaccatcaccaaccaatacaagtttgactggcctggggctntcctgcgcaagccatcgnggggttcctttcanaagcgtttccgcgccccgtccgatgaggctggaagatgggggncccgcgtcctcggccgggacccctaagccgaatntgcaatttcatacactggcggcggtcggcatgctttanagggccaattcgcctntgngagtggatacaattantggccgcgnttacacgcgngatngggaaacccgg

SEQ ID NO: 56 - DNA, 746 bp, Homo sapiens
  misc_feature (1)...(746): n = A,T,C or G
cnnnnnnncngncgcagtgtgctggaattcggcttcgagcggccgcccgggcaggtactctattctcttatccaggactggatcaaatgatttatggcattgtgctgttttaatgttctcatccacagggtcgattccaataactgaagccccaagccgccctagaggttcagttaacagcccaccaccacagccaacgtcaagaatcttcatccccaacaaaggttttcctggctggtgattaggaattgttttcagaagattgtccctaataaatggcaccctcaggtcattcatggaatgaagaggtgcatatactccttgttcatcccaccatttgtgagccagggccaagaaggtttttacctcaccgctgtcgacagtggtttgggaagtacctcggccgcgaccacgctaagccgaattctgcagatatccatcacactggcggnccgtcgagcatgcatctagagggcccaattcgccctatagtgagtcgtattacaattactgggccgtcgttttacaacgtcgtgactgggnaaaccctgggggtaccccaacttnaatggcttgcagcacaatccccttttngcagctggcgtaataacgaagaggccggacccgatcgcctttccaaagntgcncanctgaatggngaatggacgcccccttancngngcattancgcggnggtgngnggttcccncangtgaccgtacattgcaaggcctaaggccgttntt

SEQ ID NO: 57 - DNA, 749 bp, Homo sapiens
  misc_feature (1)...(749): n = A,T,C or G
nnacngncgncngtgtgctggaattcggctttcgagcggccgcccgggcaggtacatatacatttgcaaaacttgagtttatatgcagctttttaaagaacagaacacttgcttgaagtgagtttccccaaagtatctgaatgcctgccacactctcctctcccccattacatggtatttcctcacagttcagattggcaagtgcacttggggtcactccagacacataccttgctcaggagcctctgagtgcagctcctaaatatctggggcctacagcctagacaacccacttaagtgctaggtacctcggccgcgaccacgctaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcaagacaatccccttttcgncagctgggggaataacgnangaggcccggaccgatcgcccttcccaacaagttgcgcagcctgaatggcgaatggacncgcctgtagcggggcaattaagcgccgnnggggggggnggttccccncaggnngaccggttnactttgcaagggcctaacgcccgttctttggttttttccttccttttngcncgttcgccggttttcccgtaaggtttaaatgggggctccttagggtccgattn

SEQ ID NO: 58 - DNA, 735 bp, Homo sapiens
  misc_feature (1)...(735): n = A,T,C or G
tcctncangnctgcccaggtnacctgacctgagtccctgnnntnncccgntcagncncncangntgcacatgtnacaaccgagcntactggatcggtgaaagaaaataataataatgagcatctaagtggttgggttttagagatcaatcaagaataatttaattttcttttgtatttgaaatgtaaatagttttcttttcgattaaaaaaatttcctataactgctaaacagttaaaaactttaaagtagtaaatgagtttatagaaagcatgtattcttgatttttgtgccttggtaaagttgataactatttatgaatatttgaccaaattattccagcatcagaataataagcaaaataactttgttagtgttttgttagactgtttatgaaatataacaagaaatactattttgtgttggatggcggttatttttaaacttaattaatcctgtgaaaaggcctcttaccttatggtatataattaaacaaaatcnggagaaatgtatcagnttgctcaattatcaagncttgcttttgccgcttatggnnctctacaccctgtaagggttgttgaaggaaagagaactaaggaaggtntatttgctagcgttttgtaagtngcttataagnaatggggcctttcaangcttctgnggatgattgcttgtctggttctncgttaaggaaatgaacttgganaaggaagcccttttgaggtcaccgtcccnc

SEQ ID NO: 59 - DNA, 742 bp, Homo sapiens
  misc_feature (1)...(742): n = A,T,C or G
gnnnnnngtntgctggaattcggcttagcgtggtcgcggccgaggtactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatgacaggagggcagaccacattgcttttcacatccatttcccctccttccatgggcagaggaccaggctgtaggaaatctagttatttacaggaacttcatcataatttggagggaagctcttggagctgtgagtttctccctgtacctgcccgggccgggcgctcgaaaccgaattctgcagatattcatacactggccgggcgnttcgagcatgccntttagagggcccaattcgcctatagtgagtcgganttacaattaacttggccggcgttttacaaacgtcggggactggggaaaaccctggcgttacccaacttaaatcgcttgnagnacatcccctttcgccagctggngtaatagcnaaaaggccgaccgatcgnctttncaacaattgcncaccngatggnaatggacccctgtacgggcattaacccgggggg

SEQ ID NO: 60 - DNA, 736 bp, Homo sapiens
  misc_feature (1)...(736): n = A,T,C or G
gncgcagtgtgctggaattcggctttcgagcggccgcccgggcaggtacaaaaagtatctatgacactagtattaaattaaaattaattttaaattttaccacaataaatgtatacatagtataaggaaaactacagatgctttcagatcaccagtctagaacatattctgcaaagtggctaattcacattatatttttcttttaatactgagttccataaaaatgcaagttcttccattgtttgagttctgaagtcagtattcattcttgcaaccaatgaacagtgttttgagaaaacagttttctttgtaaatattccaccaattcacgtcttgattttagcatcccaatctcatgataggggatctgaacaacttgataaccgagtaactgtaggtgtctttgtttaatagcttctttccccagtaagtgtttgctattgggagcaaaacctttttggaccatcaatacacaagggctatccttttatnggatatcttcattagctgngggatggcaatacaaatccttcttcattaatttaatttcaacatctatggttaacaatagggtggcaacacttttggagcaaaatataatcntgctcctaaaaggtagtcagcccaatctttancatatntttaaagtgggaatccnangggtctccanggaccacatggggnaagaaagacttccttgtnttaggaaggngtttggncctttngaaaaggnatt

SEQ ID NO: 61 - DNA, 776 bp, Homo sapiens
  misc_feature (1)...(776): n = A,T,C or G
tnnnnnnnnnnnnnnnnnnnnnnnnnnnnnnnnnnnnnnnnntnnnnngnncgncngngtgctggaattcggcttagcgtggtcgcggccgaggtacatgttggaaaaatccttctaccagtttcagcattatagagcaattccaggagtagtagagaaggtaaagaattcagaagaacagtataataaaatagtaattcccaatgaagaaagtgtggttatctattataagattagacagcaacttgccaaattgggtaaagaaattgaagaatatattcacaaaccaaaatactgcttaccttttctacaaccaggtcgtttggtaaaggtaaagaatgaaggagatgactttggctggggagtagtggtgaatttctcaaaaaagtcaaatgttaagcctaactctggtgaactggatcctttgtatgtagtagaagtacattcttcccattcatatgtattatttaacctcagagcatttgcgggtttatacttataactctgtatagaangatattatatatnaaccgtaacattaaanggaagaatatcaagtctagtcatctccatagaactttttccccctaatcttctaaatattggtaatcaccgaaaatgtttagatgctctcatggnaattctgggttacaatttnttaaaacattcttggntnttttaaatacaccatanagggagctttccatatggttanaaaaaataaattgcctttaatgcccaaaaaaaaaaaaaaaaaaaaagtcctgccggcggcc

SEQ ID NO: 62 - DNA, 785 bp, Homo sapiens
  misc_feature (1)...(785): n = A,T,C or G
tnnnnnnnntnnnnantnntnnntnnnntnnnanacntnntnacggccgccagngtgctggaattcggctttcgagcggccgcccgggcaggtacatcccccggctgcacagactcttcagatctgttngcttgaacagggacccatagtatgtggccagtgcagggtcatctctctgaatgagaaggggcttctgctnccagaattccttgaaaaaagtctctgtcttgatgggcgagattaaactttcaaagagactactgggactatcaaagtttaaagctgaaggccccccagctgcttctaacttcatctgcttacagggagccggcccctcttccttcccactccctgtaggctttgctttctttggcatcgttctgtcttcaagacaaagcagtaaggaaatgcaaacctcaatgtattttcgtgaccatgaggttcaaagcttgatgtccaggaggcaaaaaccagtacctcggccgcgaccacgcttaagccgaattctgcagatattcatcacactgggcgggccgttcgagcatgcattttagagggcccaaattcgccctatagngagtcgtattacaattcactggcccgtggtttacaaacgtcntgactggggaaaaccctggcgttnccaacttaatcgccttgangacatcccctttcgncaggtggngtaataccgaanagcccccaccgancgcctttncaanantnccaanctgnatggcgaatggnccncccttancgcnantaagcccggnggggnngnggnn

SEQ ID NO: 63 - DNA, 741 bp, Homo sapiens
  misc_feature (1)...(741): n = A,T,C or G
tccctannaacggcccgccagtgtgctggaattcggctttgagcggccgcccgggcaggtacctttcccattgtggtcatgccatttggcagggggagaatgggaggcttggccttctttgtgaggcagtgtgagcagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcangaaccacatccccatctgttaggccttggtgaggaggccctgggcaaaaaaagggtctttcgcaaagcgatgtcaganggccgggtttgagcctttctataagctatagcntttgttatttcanccgntcacttactgnataatttaaaaacatttatgtactgagacacttttntatttcaatcatatcatgaacattttatttgcttaaacttgngtcatgggnaggctggaatatgtgaccttgggccggaccacnctaagccaatnttgaaatttcatacactggnggccgttgncatcattaanggccaatcnctattnnntct

SEQ ID NO: 64 - DNA, 785 bp, Homo sapiens
  misc_feature (1)...(785): n = A,T,C or G
cttnnnnnttnncncngcttcncctnctngttccctntacggccgccagtgtgctggaattcggcttagcgtggtcgcggccgaggtaccagaatatggacacattccaagctttcttgtcgatgcttgcacatctttagaagaccatattcataccgaagggctttttcggaaatcaggatctgtgattcgcctaaaagcactaaagaataaagtggatcatggtgaaggttgcctatcttctgcacctccttgtgatattgcgggacttcttaagcagttttttagggaactgccagagcccattctcccagctgatttgcatgaagcacttttgaaagctcaacagttaggcacagaggaaaagaataaagctacactgttgctctcctgtcttctggctgaccacacagttcatgtattaagatacttctttaacttttctcaggaatgtttttcttagatccagtgagaatangatggacagcaagcaatcttgcagtaatatttgcacccnatnttntttagacaagtngaaggacatgaaaagatgtctttntaacacagaaaaagaagcttacgaattnccggcntgcagtaagnccntgcccgngnctactttntttnnccntntctcctcnccttgntnccctntncnnncactntcnnttnntcnatctcncnnnnttccnttttntnnnctccnntccttcttncaanattnnncgnncctcactctncgnatcctncactcntcnntnanccactcntccatcnnnnctccc

SEQ ID NO: 65 - DNA, 730 bp, Homo sapiens
  misc_feature (1)...(730): n = A,T,C or G
cntnnnnncggncgncngngtgctggaattcggcttagcgtggtcgcggccgaggtacatttagaaagccaagtcgaaaaaaaaaaaagggtgaatttccaagtgccttataagaggatataatgtaaaattttctaaatgattcaagtaattatctttggtatattctagaatataccagaaacactttgacaaacacagaaacaactgggtcgtgagcattttagtgggggtaattttggattctttctccaagcattactggtaaataacaccagctaaaggcattcaaagaacagtatcttatttatgggttcacccacccctaaaaagtaataaatatggattagcagatgcacatttctcaagagaagtgaagtcagtgttatgcaggagacagtgattgtcaacttaattttatattcttcatgtttagcctccaagtccttgaggagtttacagttttaggaattgtaaacagttgcagcatattgntctgtatgactaaacttggtagcattcagagacccaaaatttaataagcncatttttntccttaanggattaaaattttgaatgngacncccagtaaaccaatcctcaaaaaaatcnaggtnttaaaagnggaaaagcctancccctacctataagccccatccccccgtacagttaatacataaccttcannatttttaggttaaattactattttntaggtccngncggcgcc

SEQ ID NO: 66 - DNA, 737 bp, Homo sapiens
  misc_feature (1)...(737): n = A,T,C or G
tccntngnncggncgncagtgtgctggaattcggcttagcgtggtcgcggccgaggtacacttcacccatgcttttaaaatgtaagcaacatttaatcttcaaaatgaaaaaatagacaaatttttctaataccaaaatataagattaaaatacttgtaaaataaaatgttagtgcgaaatagggctttttttttttacatcaaaaaggaaatatttttgacttgcttttcttctgtaaatcctcccatctcactaatatttacaacaatccagagtagcgtttatgagacactgaaaaagacagggaggaaatcctttttcaagatatgaagtcagaacctgaatgtagacatcggacagagaagtcctcaaccacaaacctgtcctccagctctagagagagtaaggctgtatttccaaccttgagatttttcattacattttcccctttttgggtgttaaattctttccaagaatgctgtacctgcccnggcggccgctcgaagcccgaattctncanatttccttacctggcggncgttcgagcatgcttttagagggccaattcgccctatagnggtcgnataccaatcactggccgtcgtttacaacgtcgngctgggnaaaccccngcggtacccaacttaatcgcctgnagcanatncccnttngccantggngtaatancgaanaggnccgacccatcgccttccaanaattggcancctgatgggga

SEQ ID NO: 67 - DNA, 913 bp, Homo sapiens
  misc_feature (1)...(913): n = A,T,C or G
tntatncctagtacggccgcagtgtgctggaattcggcttagcgtggtcgcggccgaggtactgccagagctatacacccagaggtggtgtgtggtcccaaactggatgagtggaccctacaccaacctgatgtagatgttttatagttgaatataatgaaagatatggatatagacttttattataagagctgtgcctaggtagatgagtctgtgggtagaaagtcctcaggttgtgtctgtcacatcactggtggttctagtgcttctagtctgggccctctgagacctctaatttctgcattaaattgttacataatgatagcctttgtgctgtcatgtggcttttgggggtaatggcaagtacctgcccgggcggtcgctcgaaagccgaattctgcaatatccatcacactggcggccgctcnagcatgcatctagagggcccaattcgccctatagtgagtcgtattacaattcactggccgtcgttttacaacgtcgtgactgggaaaaccctgcgttcccaacttaatcgccttgcacacattccctttcgccagctggcgtaatagcgaagaaggcccgacccgatcgncctttccacagttgcgcaccctgaatggtgaatggacgcnccctgtancggcgcattaagcgccggcggtgtggtggttacnccnaggtgaccgtncacttgccacgcctaacgcccgtcctttgtttnttccttctttttcncccgtagcggtttncccnnanntntaatgggctccttaggtccattantgttacgnnctcncccaaaanttnttgggangntactatggcntccctaaacggtttccttgngtggaccnttntantnggcttttcaatgaaaccnacntttggntttttttaaggattcnng

SEQ ID NO: 68 - DNA, 908 bp, Homo sapiens
  misc_feature (1)...(908): n = A,T,C or G
ancntntacggncgnngtgtgctggaattcggcttagcgtggtcgcggccgaggtactcaggtatgaaaagaggaaacatgttatatcagtcatataaaattccagaaaattgacattaatctactgttaagcaaatattgtaattcagtgattgtctggagcatggagaggccagagagaaagatagtatttattttttaagagacagggtcttactatgggacccaggctagtctcaaacttcttacctcaagcaatactcccaccctggactcccaaagtgttgggataacaggcatgggtcaccatgcccagtcttattacagctttaaaaatttaatttgaaagaactatctggatttaacattatttttgacaaataggttatgtgattttacattgactctcctaactctnatcacgccctaaggggggcaattagcctgtgtgataccaaatacttatgagccaggaagatcattttcacctgattaaattggaatgctggctggttgcaaaggcccatcactggactaagagattcctttctcctatcctcttcctcctctaanttaaggatggtttttggtatgcagcccatataggctcagaaatccacaaancttagatttagtctgccatgacctaagctacactagatcataatatggagggcnnaaaattntntnccnccccggcgggccgntcaaagcccantntgagatttcttacactggcggccgttgancatgcattanaggcccattcnctntanggtngatacaatactggccgcnttacacgcgggcgggaaacctgggttccactattgcttaganacccttnncgtggtannnaaagccnccccncctcccantgccccaangaagccccttgnntancgngg

SEQ ID NO: 69 - DNA, 914 bp, Homo sapiens
  misc_feature (1)...(914): n = A,T,C or G
cnatccctagtnncggncgcagtgtgctggaattcggcttagcgtggtcgcggccgaggtacttcctctgtaaattatctaaacgtgcttgaactcttttagcctgaatgtctatcttcctgttttcttcacttgcttcatttaactgtttttttaaggtatcattcaattctttcaatgcatcaaaagaggacctcagtctcttggtttctttattctttnccttaagaacttcggttaagtgttcaacttctgcatcatgctgctcttttataatttgaaatcttgtaagaacctcttcttcaacacctttaattttactcaaggcattttcatgtctgaaaagcaagttgttctctttcaagtnaaaaacgntctcgcttttgaagttcttcagcaaatttctgttgtgctgcagtttcacactgcaatttcagatgtatctgaaataattcgtcatatatctgactgatctgatgtggaagtaaactccgcttgtctgatgctttagattttggacagctgcaggtttcaaaggagagtcacctgcatcactacaacacttcagttccgaggtctcgatgaggncgtattagttggcaaggcaccttttctatttctttaaaagactgcttgggtaattttcgaggacctgcccggcnggcgctcgaaagccgaatcttcagtatcntccactggcggccggtcgagcatncattanagggcccaatcgccnntagngagcgtttacaatnnttgccgcgtttccacgtnggctnggaaacccgngtaccacttaatncttgancnatccctttccagtgngtannanaaangccnccatcncctccanatgcnccgaggaaggcccccccngcntaancgggnggggtnccnnccccncccccctccct

SEQ ID NO: 70 - DNA, 918 bp, Homo sapiens
  misc_feature (1)...(918): n = A,T,C or G
cggatccctagtaacggccgccagtgtgctggaattcggcttagcgtggtcgcggccgaggtacaagacttgaaatgtttagttttcagtcttattggattacacttcaagattaaaccaatttaaattgtatgttttcaggctgtttgtatatttaattaagggatgggaggggttatttgtcatttacagtattggggtttttatgaatgtgaagcaaacaaaaaaaatttgtatgtaaactgaaaataagaaaatacattagcaagcttaatggttatccttacttgagtccatatgggttggacagtccccacacacattaaattctgtaaatgaaagccaccttttgttaaaaatttgctctaataaaacataccaaatccacntnaatanacnntancttanaaagtacccttncccggncgnattatnncttcaatctctttnncataatttcacntanttgtctcttctttntntttccatnctctnntctctctttcttttattccncccttntntgantttactccttctctaccncnccnccnaaattcccaattttttttctttcttctttattnnttaaacncttatctttnactcttantnccaatagttnaattcttttctnnttanantttntntcanntcactgtcnnccctttnntntnnatnctcntcatattatnttaancnttctnntntcattattccccttctctttttgctcctatnnnccttcattantatcntatcnttcntcatatcatacnnnncnacttntcncnatntattantctatnnttaanttcatcttcncntttatcnacactcncntnttnncntnctnatanttntntttnntcatcctcntntatttnaattntntcctatcnttatntncntcttnng

SEQ ID NO: 71 - DNA, 896 bp, Homo sapiens
  misc_feature (1)...(896): n = A,T,C or G
tnannantnnnacggncgcagtgtgctggaattcggctttcgagcggccgcccgggcaggtacgcggggacatatctgcttccgacacagctgcaatcactagcaagctctcaggcctggcatcatggtgcattttactgctgaggagaaggctgccgtcactagcctgtggagcaagatgaatgtggaagaggctggaggtgaagccttgggcagactcctcgttgtctacccctggacccagagattttttgacagctttggaaacctgtcgtctccctctgccatcctgagcaaccccaaggtcaaggcccatggcaagaaggtgctgacttcctttggagatgctattaaaaacatggacaacctcaagcccgcctttgctaagctgagtgagctgcactgtgacaagctgcatgtggatcctgagaacttcaagctcctgggtaacgtgatggtgattattctggctactcactttggcaaggagttcacccctnaagtgcaagctgcctggcaaaagctggtgtctgctgtcgccantgccctggcccataagtaccttcggncgcgaccacgctaagccgaaatctgcagatattcatcacactngcggccnttgacatgcatctagagggccaattcccctatatgagcgtattacaaatcactggncggcttttacaactcgtgnttgggaaacctggcgtaccacttatcgcttgagacatccctttgcanctnggtataccnaaagcccncatcgccttcanattncaacctatggaatggccccctnacggccttacccggggtggggtncccaatnacntcattcaccctnncctcttttttctttttncaatnggttccaaantaaanggcntnggcntaag

SEQ ID NO: 72 - DNA, 908 bp, Homo sapiens
  misc_feature (1)...(908): n = A,T,C or G
atnnctnnannctcncagantgctggaattnggnttagcgnggtcgcngccgangnacagnagnggnatatctagccgccacctcttcaaaaatccatggcctggactggcacccanacagcgagcacattcttgctaccttcagtcaagacaattntgngaagttctgggattaccgccagcctcggaaatacctcaatattcttccttgccaggtgcctgtctggaaggccagatacacacctttcggcaatggattggtgactgtgatggttccccagctgcggagggaaaacagccttctctgtggaatgtctttgacttgaacaccccantncacaccttcgnggggcatgatgatggggcctggagttcagtggaggaagcataangaagggtccaaggactatcaactggngacgtggtccgggatcagaccttgagaatgtggtgggtggattcccanatgcaaangctttgtgcaaangacatattatacggtgntgatgaagtcattgagagtattttcctttttgccggnacctnagaaggccctgcacactgaatatacagatcaccatnacacttgcaacctggggaggaagagccctaaaagaaaaccccctanaantctnntggaaaaaagnaataaancaatggggntngcctanaccttgcanangaatttttcttatcatntccaatccgaaantnatttgtnatggtgcggataaggactnacatgttntcctgacaacntctggcaanngtgggaantccttgnaaanctccgggggcgtnaaaccaatttnatatcaccacngngcgtcnnatctctangggcaatnccntatggtgttcatcttgggggttaaccgncggggaantgntccatttccttggacccg

SEQ ID NO: 73 - DNA, 878 bp, Homo sapiens
  misc_feature (1)...(878): n = A,T,C or G
tnactaatnccgncgncttgtgctggaatnnnnctngcgagggcccggncgaggttccttgngnangagcaanannctcaccngttaancagnntccaggttatcaaanggnactggtngtccangtggnnggnnngggnggccnaccacnnnctcaaattnccnntctgnntntcctnnctcgntntggactnntngtnanggggtcnngngntnntattgctnnnntngccnnctttgngnangggctngngntaacccnggggcctnncttttatggggangttatgnacnnnatcggatagaccccntnnnntttcttcaanatagtcangctttcagnccnntcctccnaatgaaaacccaggtgantanaagtccatggggtngatggcatccnctcctttgctttgangatttngaagttcnctctctntgacngaaaaangggntttncaaggggaannctctcncntnangggtcacgattgcntannntgacaatgtttnactntgaaccctagnacncttctccnatattcatntcttgggnctttgcncanaagacggggnnatcccttcncttannggtgcgtgggccaatcttgngcaccnntttttttggcntnagntttnccttgggaaancaaccctttnnancnttaattgggccanaaggncccntccaccgcccnccgngtntatgttattgntgncgaannccattttgctccgggccngggngtanacttntnngaaaggaccaaccatgccttcttctgaantgggnttatttttcacnagccagggaaatntntccccaaanttttttntttntntttngcnnactgcaaattanttcntggagtaccaancnn

SEQ ID NO: 74 - DNA, 932 bp, Homo sapiens
  misc_feature (1)...(932): n = A,T,C or G
ttnannncnncnntnnnntntnacnnncgtcantgtgctggaattcggctttcgagcggccgnccgggcnggtacgcacagncgtctcagaagccagatacaaatgttagtcagagagaacacagacctccgggaagaaataaaagtgatggaaagattccgactggatgcctggaagagagcagaagccatagagagcagcctcgaggtggagaagaaggacatgcttgcgaacacatctgttcgatttcaaaacagtcagatttcttcaggaacccaggtagaaaaatacaanaaaaattatcttccaatgcaaggcaatccacctcgaagatccaagtctgcacctcctcgtgatttaggcaatttggataagggacaagctgcctctccagggagccacttgaaccactgaacttcccagatcctgaatataaagaggaggaggaagaccannacatacagggagaaatcantcatnctgatggaaaggnggaaaaggtttataagaatgggtgccgtgttatactgtttccaatggaactcnaaaggaagtgagtgcngatnggaanaccatcactgncactttctttaatggtgaccgtgnagcaagtcatgccntnaccaangagtgatctactctatgcccttgccataccactcacacgacattcccggggggactggagccttcaattcttaagtggncaaattnanacattcccaatnganaaaagaatcacgttcttgncagctnttaaactatttcttgntgcaataagaanatttcctatggaccttgccgnaccctaaccnatttttaattcntaacttgggcgtcgcatcttnagggcaattcctagggggttcattatngcggttacggggnggaaaccnggtccatttctggcatccttccgggnnaaaaaccg

SEQ ID NO: 75 - DNA, 899 bp, Homo sapiens
  misc_feature (1)...(899): n = A,T,C or G
tnntctnngnnacngncgcagngtgctggaattcggctttcgagcggccgcccgggcaggtacgcgggaggtctctgtcgagcagcggacgccggtctctgttccgcaggatggggtttgttaaagttgttaagaataaggcctactttaagagataccaagtgaaatttagaagacgacgagagggtaaaactgattattatgctcggaaacgcttggtgatacaagataaaaataaatacaacacacccaaatacaggatgatagttcgtgtgacaaacagagatatcatttgtcagattgcttatgcccgtatagagggggatatgatagtctgcgcagcgtatgcacacnaactgccaaaatatggtgtgaaggttggcctgacaaattatgctgcagcatattgtacctcggncgcgaccacgctaagccgaattctgcagatattcatcacactggcggncgctcgagcatgcatctngagggcccaattcgccctatagtgagtcgttttacaattcactgcccgtcggtttacaacgtcttgactgggaaaaccctgcgttacccaacttaatcgcctttgcagnacatcccctttcgncagctggcgtaataacgaananggccnntnccgatncgcccttcctaacaagttgnncngcctgatggcnaatggacgcccctgtaccgngcataacgccgcngtgtgnggtaccncangtgccgtnattcaaggcctnggccgtctttgttttcctcttttgccttggggtttccnnagttaangggctcntangtccttatgtncggnntaccaaantgtnngntgnnnatggctcccnaanggttccctaactgccattatagnnnttnaagaantanttttntttttt

SEQ ID NO: 76 - DNA, 884 bp, Homo sapiens
  misc_feature (1)...(884): n = A,T,C or G
tnnnnnnntttaacggccgcagtgtgctggaattcggcttcgagcggccgcccgggcaggtaccctctgctatccatccacacacagtgaaatggatgggagggatgagggaacaacacatcatgggagacaaagggaagaccaaggaaagtgctggcagtctccgcttttccattcagaatggatggaactgggcccgttagtggcactaagacagggcagggctatttttttttttaaataaaatattttaaagatgcataaaatggacaccttagcacgtgagtgtcctttgggctcttcaagacatcttctttaaggtttacccctggagtagcagagggtctcctcacccaggtggaaagagatgggctccatctgcagatgaaggaaaggcactggccgcacagcactcagcaatgtagatacaggcaccangtangagcaggatggcttacccagcaacctgcctcttagctcctgctagtgttcttcctcctcccaagtttcagttaaaagtacctcggccgcgaccacgctaaccgaattctgagatatccatnacactggcggccgttcgagcattcatntagagggcccattcgccctatagtgagncgattacaaattactngccgcggtttacacgcnngctgggaaacctgngtaccacttatgcttgannnatcccntttgcagtgggtatacnaaagcccccgatcccttccaaattccacttatggaatgaccccctttcngccntnannngggntngngttccaagggcgttctttcagccctagcctttttttttcttttttcnttgcgttcccnaattaanggnncttgntnttattttcgcccccaatttnnggtcnnggcct

SEQ ID NO: 77 - DNA, 868 bp, Homo sapiens
  misc_feature (1)...(868): n = A,T,C or G
cctngtacggncgcagngtgctggaattcggctttcgagcggccgcccgggcaggtactctgcataagggggaaaagcttccagaaagcagccatgaaccaggctgtccaggaatggcagctgtatccaaccacaaacaacaaaggctaccctttgaccaaatgtctttctctgcaacatgggcttcggcctaaaatatgcagaagacagatgaggtcaaatactcagttggctctctttatctcccttgcctttatggtgaaacaggggagatgtgcacctttcaggcacaagccctagtttggcgcctgctgctccttggttttgcctggttagactttcagtgacagatgttggggtgtttttgcttagaaaggtccccttgtctcaccttgcagggcaggcatgccagtctctgccagttccactgcccccttgatctttgaaggagtcctcagcccctcgcggcataaggatgttttgcaacttttcagaatctggcccagaaattanggctcaatttcctgattgtagtagaggtaaaaatgctgtgagcttatagataaaaaccgagagaagtagctgggtcttgnattccttgggngtggnggaataacaggggaatttgaacaaggagagnaaaaagggatttggtttntggggggggggtttttctaggtttgccngtgggtttaaggagaanaaatgcaatcntggccggncccttaccaattnnaantcatcancngggcgtcgganattaaggccatcccntaggngntaaanantgcnggttnacctggangaaacgggcantaatttgnnaccttcccgnggaagaagccccnctcaatcccntnnggagccc

SEQ ID NO: 78 - DNA, 884 bp, Homo sapiens
  misc_feature (1)...(884): n = A,T,C or G
cnnctnnntccctntacggncgcagtgtgctggaattcggcttagcgtggtcgcggccgaggtacagtgtatgaaggcactcctggtgcccagttccccagaatgtccagttagcatctgcacatttggcttgatacatttatacatttataaaaaactctgtggtcttagagttcctgctactcttaggcctggcccagctcaaccagagaaggggttcctcctgtgtcggattccctgggagagaggatctgccttggactcaagggtccagaaggtccaggggcacattcaggattgggcctgtgagaagaaatcccagaactctgtaacctgccaccaaagcttcttacattatggaggggaaagggggaaggaacaagttttctttaaaaaaatttttttttcatacaaaaatagatccatttgcaaaacaatttctcagccaggaggctccacctcccatttnctttgtagacaagaggggtgaggtggttgggatggtcacaagtacctgcccgggcggnccgctcgagcatgcatntagagggccaattcgccctatagtngtcgtattacaaattactggncgtcgntttacaacgtctgactgggaaaacctgcgtacccaacttaatcgcnttgaagacattnccttttnnngctgcgtaatacgaaangcccnaccgacgccntncacanttgncacctgatggnaaaggncccccctttcgncataaccngggntngggtccccagtggccntnattcaagccttgccgtnttttttttctttttcnatgcggttcnnagttanngggtccttngtnnnttgttcgncccccaanttanggnggnnngnggcncccnaagttncttngnnc

SEQ ID NO: 79 - DNA, 888 bp, Homo sapiens
  misc_feature (1)...(888): n = A,T,C or G
ctnnnatccctagtacggccgncagtgtgctggaattcggcttagcgtggtcgcggccgaggtactttttttgtttttgtttttgtattttttttttcttttgaaagggtttgttaatttttctaattttaccaaagtttgcagcctatacctcaataaaacagggatattttaaatcacatacctgcagacaaactggagcaatgttatttttaaagggtttttttcacctccttattcttagattattaatgtattagggaagaatgagacaattttgtgtaggctttttctaaagtccagtacctgcccgggcggccgctcgaagccgaattctgcagatatccatcacactggcggccgctcgagcatgcatctagagggcccaattcgccctatagtgagtcgtattacaattcactggccgtcgttttacaacgtcgtgactgggaaaaccctgcgttacccaactttaatcgccttgcagcacatccccctttcnccagctggcgtaataacgaaaaggcccgcaccgatcgccttccaacagttgccacctgaatggcgaatggaccccccctntngcggggccttaancccggnggttgtgtgggttccccccagnggaccgttacacttggcagngccctaacgcccggtcttttggttntttccttcntttttgccncgtnnccggtttcccgaaagtttaanaggggnnccttagggtcncattaggtttccggncccccccaaaaattnttgggngngtccgatggcccccccntaangttttcctttngnggccctntttaanngcnttttaaanaancaacccttcgttttttttaagggtnnctnccctgtaaanggtaaaaancnantant

(19) United States
(12) Patent Application Publication
(10) Pub. No.: US 2004/0076978 A1
(43) Pub. Date: Apr. 22, 2004

(54) Method to identify genes associated with chronic myelogenous leukemia

(76) Inventor: Catherine Verfaillie, St Paul, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/416,907

(22) PCT Filed: Nov. 14, 2001
(86) PCT No.: PCT/US01/43781

(51) Int. Cl. C12Q 1/68

(52) U.S. Cl. 435/6

(57) ABSTRACT

A method to detect genes that are differentially expressed in chronic myelogenous leukemia is provided.

### CROSS-REFERENCE TO RELATED APPLICATIONS

This application claims the benefit of the filing date of U.S. application Serial No. 60/248,403, filed Nov. 14, 2000, under 35 U.S.C. § 119(e).

### STATEMENT OF GOVERNMENT RIGHTS

The invention was made with a grant from the Government of the United States of America (grants ROl-HL-49930 and ROI-CA-74887 from the National Institutes of Health). The Government may have certain rights to the invention.

### BACKGROUND OF THE INVENTION

Chronic myelogenous leukemia (CML) is a lethal disease of hematopoietic stem cells, characterized by a specific chromosomal translocation between human chromosome 9 and human chromosome 22. The chromosome resulting from this translocation is commonly referred to as the Philadelphia chromosome (Darnell et al., 1990). The c-abl gene (ABL), a tyrosine kinase thought to be involved in growth control, resides on the distal arm of human chromosome 9, while the c-bcr gene (BCR) resides on human chromosome 22. The translocation places the promoter distal three exons of ABL, including those elements which encode the tyrosine kinase domain, downstream of either the first or second exon of BCR (Chung and Wong, 1995). The product of the translocation between human chromosome 9 and human chromosome 22 is a chimeric gene, BCR-ABL, which encodes a fusion protein, often referred to as p185^{BCR-ABL} or p210^{BCR-ABL}, depending upon the inclusion of the second exon of BCR (Bartram et al., 1983). p185^{BCR-ABL} causes acute leukemia, typically lymphoblastic; p210^{BCR-ABL} usually causes CML, but can occasionally also cause acute leukemia.

Following the chromosomal translocation between chromosomes 9 and 22 within a single, primitive myeloid stem cell, the progeny of the affected cell gradually populate the entire intermediate and late hematopoietic maturational compartments. Despite the presence of the Philadelphia chromosome, these progeny, referred to as Ph⁺ cells, are able to differentiate and mature along the various myeloid lineages while retaining the capacity to function as their normal, unaffected counterparts.

Clinically, CML is characterized in its initial chronic phase by the circulation of malignant progenitors in the peripheral blood (Kantarjian et al., 1985). After three to five years, the disease transforms into a blast crisis, in which presumed additional genetic abnormalities prevent an early myeloid or lymphoid progenitor from differentiating (Clarkson and Strife, 1993; Daley and Ben Neriah, 1991; Deisseroth and Arlinghaus, 1991; Sawyers et al., 1991). In the chronic phase, the pool of malignant progenitors and precursors is also massively expanded (Kantarjian et al., 1985). It is thought that the massive expansion of the malignant cell population is partly due to decreased cell death (Cotter, 1995) and to the fact that, in contrast to normal progenitors (Clarkson et al., 1997; Verfaillie et al., 1997), CML progenitors are never quiescent and proliferate continuously.

Compared with the native p145^{ABL} protein, which is found mainly in the cell nucleus, p210^{BCR-ABL} is located exclusively in the cytoplasm (Verfaillie et al., 1997; Verfaillie, 1998). The tyrosine kinase function and F-actin-binding function of p210^{BCR-ABL} is significantly elevated compared with that of the native p210^{BCR} protein (Konopka et al., 1989; Verfaillie et al., 1997; Verfaillie, 1998). The increased kinase activity and cytoplasmic location of the BCR-ABL gene product are essential elements of its transforming abilities (McWhirter et al., 1991).

Several in vitro and in vivo studies demonstrate that the presence of BCR-ABL is necessary and sufficient for transformation (Daley et al., 1990). For example, the introduction of BCR-ABL cDNA into hematopoietic cell lines causes growth factor-independent growth in vitro and tumorigenicity in vivo. Moreover, transplantation of murine stem cells transduced with BCR-ABL cDNA causes a CML-like phenotype, and transgenic expression of BCR-ABL causes a syndrome with myeloproliferative or acute leukemia-type characteristics.

Although a causative role of p210^{BCR/ABL} for the pathophysiology of CML has been demonstrated in cell line models and animal transplantation models, the mechanism(s) underlying p210^{BCR/ABL}-mediated transformation and how it causes the characteristic features of CML remain unclear.

Clinical treatment of CML has remained essentially unchanged for many years. Treatment of Ph⁺ CML with intensive chemotherapy alone does not induce persistent cytogenetic remissions (Kantarjian et al., 1985). With the exception of marrow ablative chemotherapy and/or total body irradiation followed by allogeneic bone marrow transplantation, no effective cure has been developed for the disease. And although allogeneic stem-cell transplantation can be curative (Enright et al., 1997), this therapy is available to <40% of patients because the disease commonly affects patients over the age of 50 (Kantarjian et al., 1985). Autologous transplantation is being considered as an alternative therapy. There is evidence to suggest that a Ph⁻ state can be induced by this treatment strategy and that autografting might increase survival (Bhatia et al., 1997). Unfortunately, almost all patients will suffer leukemic relapse after autografting, partly because of disease persistence in the host after the preparative regimen (Pichert et al., 1994) and partly because of persistent disease in the graft (Deisseroth et al., 1994). Novel therapeutic approaches are therefore needed desperately.

What is needed is a systematic method to identify genes associated with p210^{BCR-ABL}-mediated transformation, e.g., so as to identify therapeutic targets other than p210^{BCR-ABL} for CML.

### SUMMARY OF THE INVENTION

The invention provides a method for the differential isolation of nucleic acid sequences that are present in one nucleic acid population and not in another. The method is based upon employing a first population of cells which express a gene product that is associated with a particular phenotype or disease, and another (second) population of cells which does not express that gene product. The method comprises contacting nucleic acid from a first sample with nucleic acid from a second sample under hybridization conditions so as to form binary complexes. Preferably, the first sample comprises nucleic acid from cells which express a protein that is associated with malignancy, e.g., hematopoietic stem or progenitor cell malignancy, and the second sample comprises nucleic acid from cells which do not express the protein. Then a nucleic acid molecule is identified which is present in the first sample which is not present in the second sample. Preferably, the identified nucleic acid molecule is isolated and characterized.

In one embodiment of the invention, the first population of cells comprises a vector that encodes a first gene product such as a chimeric protein, e.g., p210^{BCR-ABL}, p185^{BCR-ABL}, p230^{BCR/ABL}, TEL-ABL, PDGF-ABL, AML-ETO, PML-RARα, MDS/EV1 and the like. Preferably, the vector also comprises a marker gene which encodes a second gene product that is detectable. In this embodiment of the invention, the second population of cells preferably comprise a vector comprising the marker gene. Cells from each population that express the second gene product are selected or detected, e.g., by sorting in a FACS, and nucleic acid molecules are identified that are preferentially expressed in the first population. The identified nucleic acid molecules, e.g., isolated RNAs or cDNAs, are then characterized, e.g., by sequencing. Thus, the method of the invention can be applied to identify genes associated with disease. However, the nucleic acid molecules of the invention are not limited to those identified by any particular method.

To define if and how p210^{BCR/ABL} affects expression of downstream genes, subtractive hybridization was employed to identify transcripts that are differentially expressed in human CD34⁺ cells that express p210^{BCR/ABL} relative to human CD34⁺ cells that do not express p210^{BCR/ABL}. Cord blood (CB) CD34⁺ cells were transduced with an MSCV-retrovirus vector containing either eGFP alone (eGFP) or BCR/ABL cDNA-IRES-eGFP (p210-eGFP). GFP⁺ cells were FACS selected (>90% purity), and subtractive hybridization performed between the two populations using the Clontech PCR-Select™ System. Seventy-nine cDNA clones that were expressed in p210-eGFP but not eGFP transduced CD34⁺ cells were sequenced and analyzed. Forty-one of the sequences did not encode a characterized human protein. Of these, at least 15 were closely homologous to expressed sequence tags (ESTs), at least 10 had homology to genomic clones for which no mRNA transcripts or proteins had been identified, while 1 sequence had no match in available genomic, RNA or protein databases. In addition, at least 22 sequences had significant homologies to known genes. These genes are involved in protein degradation, signal transduction, cell cycle regulation, and in RNA splicing. Interestingly, genes hypothesized to be downstream molecules for p210^{BCR-ABL}, i.e., c-myb, c-KIT, c-rav and c-myc, were not identified by the method of the invention. The observation that 4 differentially expressed genes (SRPK1, Sty, Gu, SNRNP-G) are involved in RNA splicing is remarkable, and may provide an explanation for the finding that CML CD34⁺ cells and cell lines express a number of alternatively spliced proteins, including Pyk2, beta₁-B integrin, CSCP, and MPP1 (Verfaille et al., unpublished results; Deininger et al., 2000).

The invention also provides isolated nucleic acid molecules comprising nucleic acid segments encoding polypeptides that are expressed in cells that express a protein, e.g., p210^{BCR-ABL}, that is associated with disease. For example, the invention includes isolated nucleic acid molecules comprising an open reading frame comprising any one of SEQ ID NOs:1-79, or the complement thereof, or nucleic acid molecules which hybridize thereto, e.g., under moderate and/or stringent hybridization conditions. Preferred nucleic acid molecules comprise an open reading frame comprising any one of SEQ ID NOs:1, 3-4, 11, 14-17, 19, 22, 25, 27, 29, 32, 35, 37-39, 41, 44-47, 52-54, 57-58, 60-62, 64, 67-79, the complement thereof, or nucleic acid molecules which hybridize thereto, e.g., under moderate and/or stringent hybridization conditions. Moderate and stringent hybridization conditions are well known to the art, see, for example sections 9.47-9.51 of Sambrook et al. (1989). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS.

The sequences of the nucleic acid molecules are useful as probes, to obtain full length sequences, i.e., a sequence that comprises an open reading frame that encodes a full length polypeptide, and in expression cassettes, as well as to prepare primers (oligonucleotides) for amplification or non-amplification-based methods to detect expression of the corresponding genes, e.g., using RT-PCR or linear amplification, in cells, such as in primary CML cells or CML lines, relative to normal cells.

Thus, the invention also provides probes and primers comprising at least a portion of the nucleic acid molecules of the invention. The probes or primers of the invention are preferably detectably labeled or have a binding site for a detectable label. Preferably, the probes or primers of the invention are at least about 7, more preferably at least about 15, but less than about 200, more preferably less than about 50, contiguous nucleotides bases having at least about 80% identity, more preferably at least about 90% identity, to the isolated nucleic acid molecules of the invention. Such probes or primers are useful to detect, quantify, isolate and/or amplify DNA strands that are related to the nucleic acid molecules of the invention.

Also provided is an expression cassette comprising an open reading frame comprising any one of the nucleic acid molecule of the invention operably linked to a promoter functional in a host cell, as well as a host cell, the genome of which is augmented with the expression cassette. Preferred host cells are vertebrate cells, e.g., mammalian cells. Further provided are isolated polypeptides encoded by the nucleic acid molecules of the invention.

The expression of partial or full length sequences corresponding to the nucleic acid molecules of the invention in sense or antisense orientation may be employed to further characterize the role of the encoded gene(s) product in diseased and/or in normal cells. Thus, overexpression or aberrant expression, e.g., expression which is spatially or temporally different, of one or more of the identified genes, may result in identifying the mechanism associated with transformation, e.g., p210^{BCR-ABL} transformation, and identifying other molecular target(s) for therapy. Once a target is identified, agents that interact with that target, including RNA or polypeptides, are employed to inhibit or prevent disease. For example, antisense sequences or ribozymes specific for the RNA target, or agents that bind to the encoded polypeptide and inhibit its activity, may be used to inhibit or prevent disease.

In a preferred embodiment of the invention, a PCR-based subtractive hybridization method is employed to identify genes, the expression of which is altered in cells that express a gene associated with hematopoietic stem cell malignancy. Those identified genes are useful as probes or primers to detect expression of those genes in malignant and normal cells, as well as therapeutic targets, e.g., via antisense expression or agents that alter the activity or amount of the encoded gene product.

Also provided is a method to identify an agent that inhibits or reduces the expression of a gene associated with hematopoietic cell malignancy. The method comprises contracting a cell or cell extract thereof with the agent, wherein the cell expresses a nucleic acid molecule comprising any one of SEQ ID Nos: 1-79. Then an agent is identified that inhibits or reduces expression of the nucleic acid molecule or the polypeptide encoded thereby. For example, the agent may a ribozyme, DNAzyme, antibody, e.g., a polyclonal, monoclonal, humanized or ScFv antibody, or antisense molecule. Preferably, the agent inhibits or reduces cell migration, cell proliferation, cell death or genetic instability, or increases cell adhesion.

### BRIEF DESCRIPTION OF THE FIGURES

FIG. 1 shows a schematic of a method to identify a gene, the expression of which is altered in p210^{BCR-ABL} cells.

FIG. 2 depicts the sequence identifier number associated with each sequence identified by the method of the invention. A) Sequence identifier number associated with the genes identified in Example 1. B) Sequence identifier number associated with the known genes identified in Example 1. C) Sequence identifier number associated with the unknown genes identified in Example 1.

FIG. 3 shows the nucleic acid sequences identified by the method of the invention.

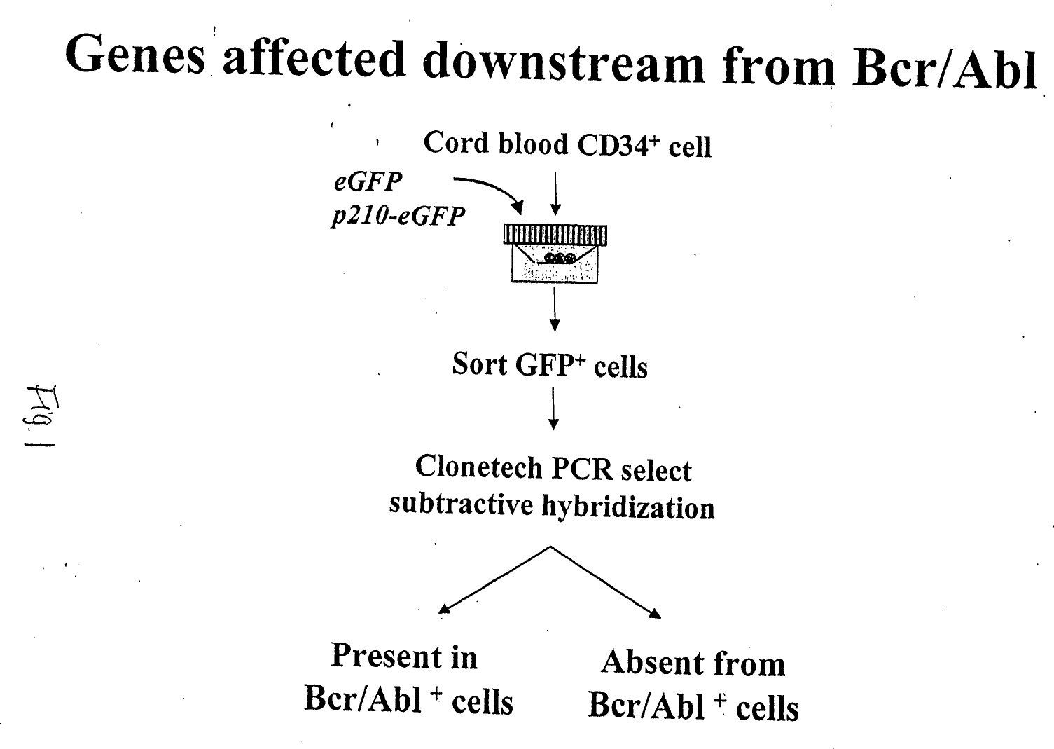

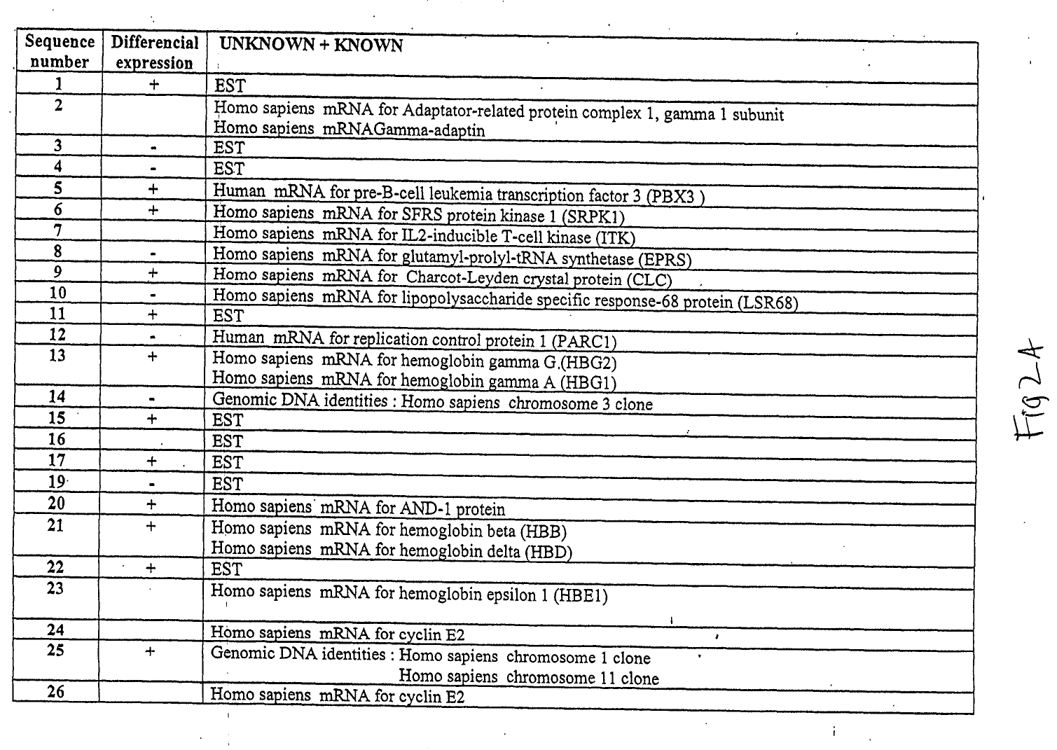

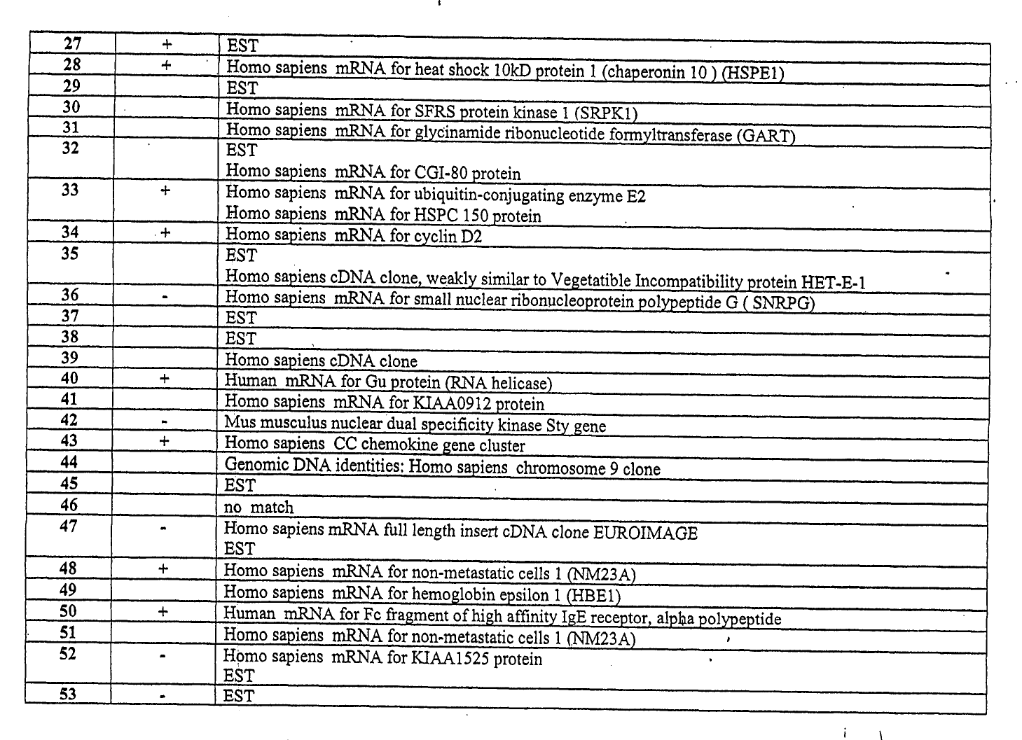

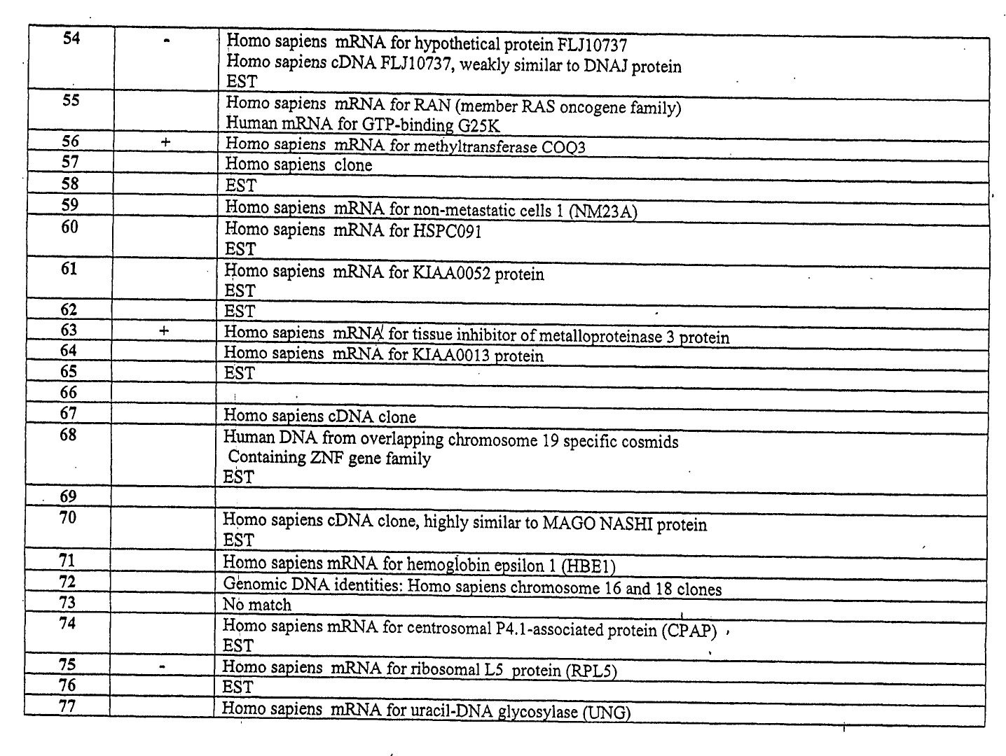

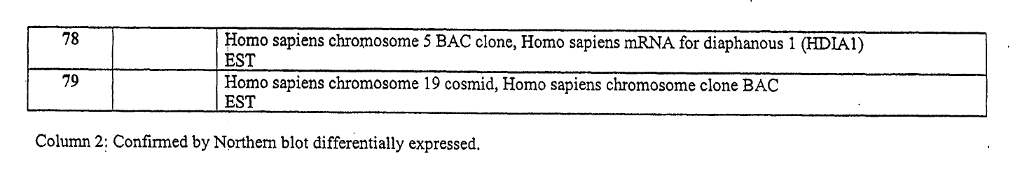

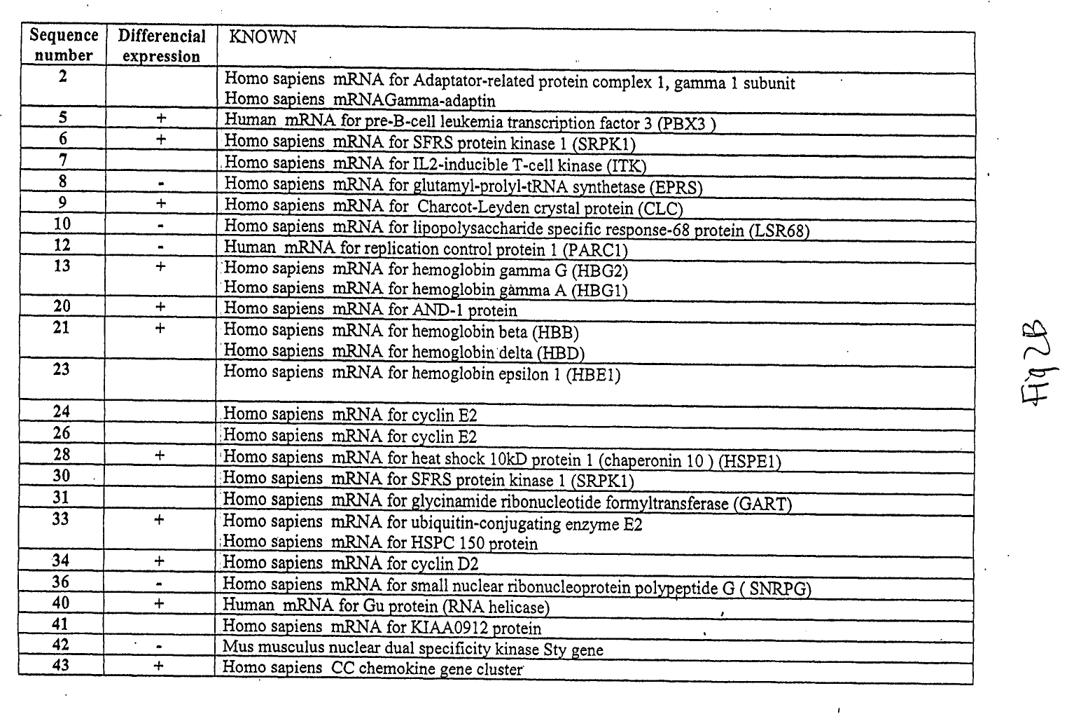

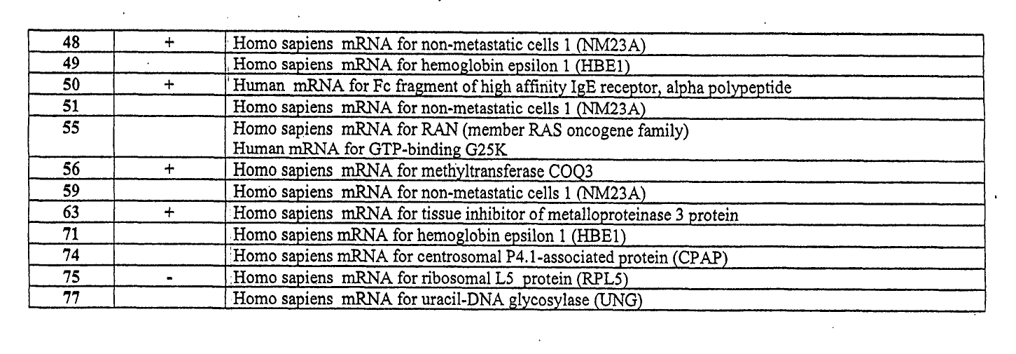

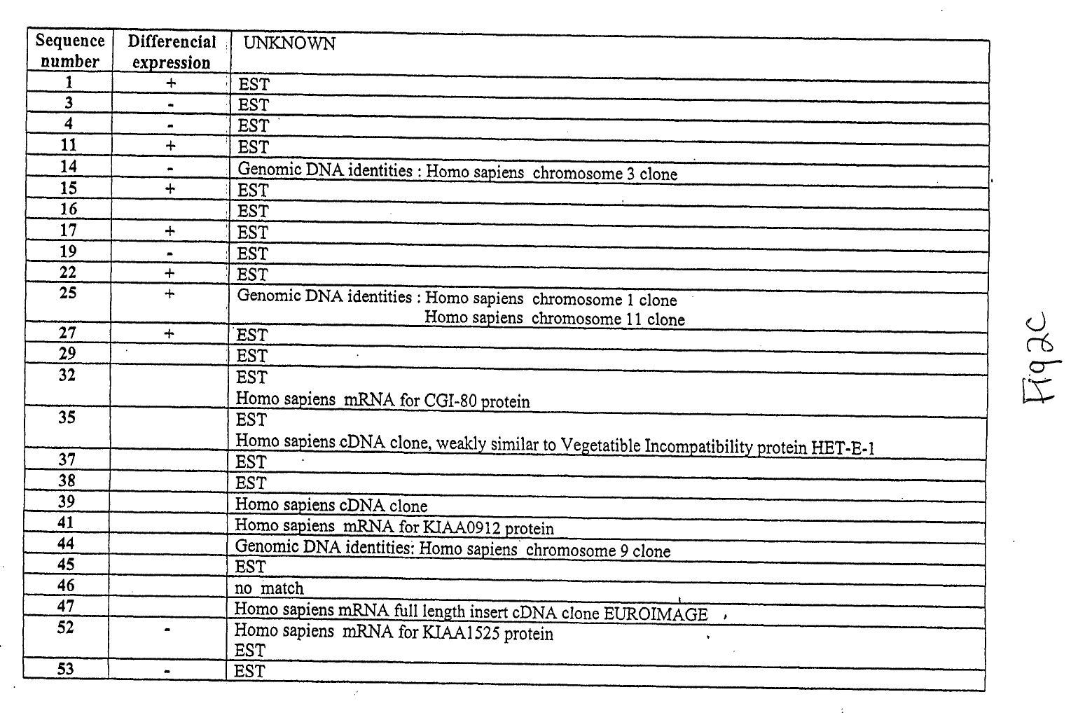

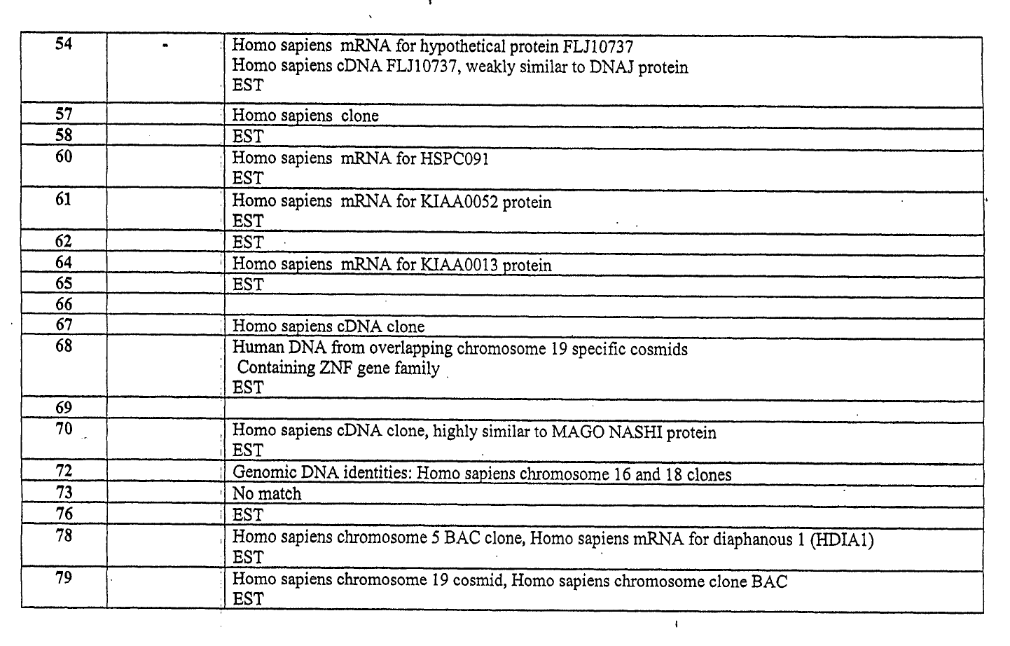

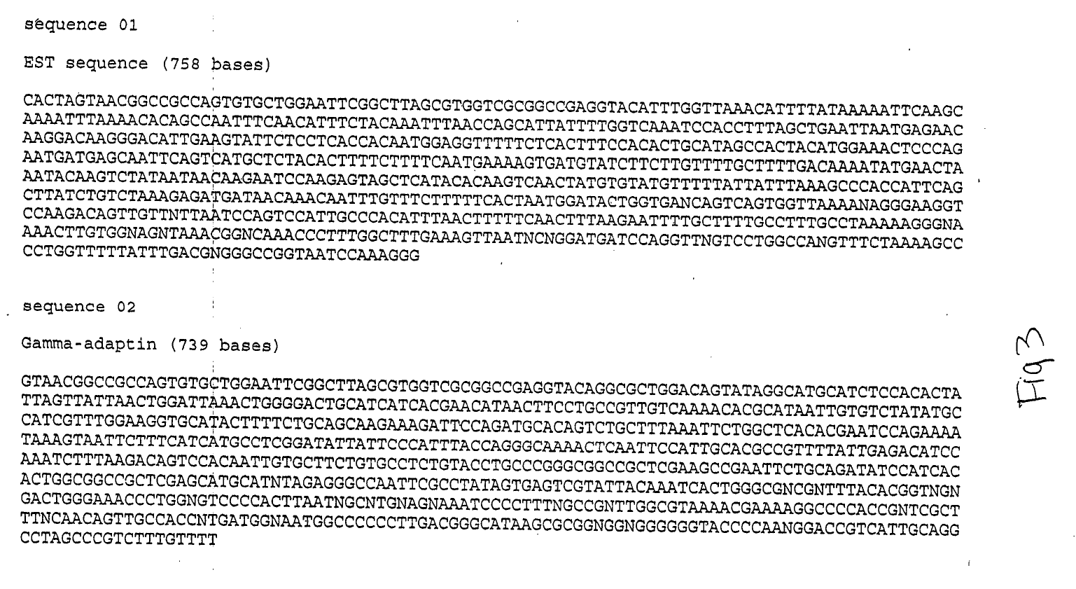

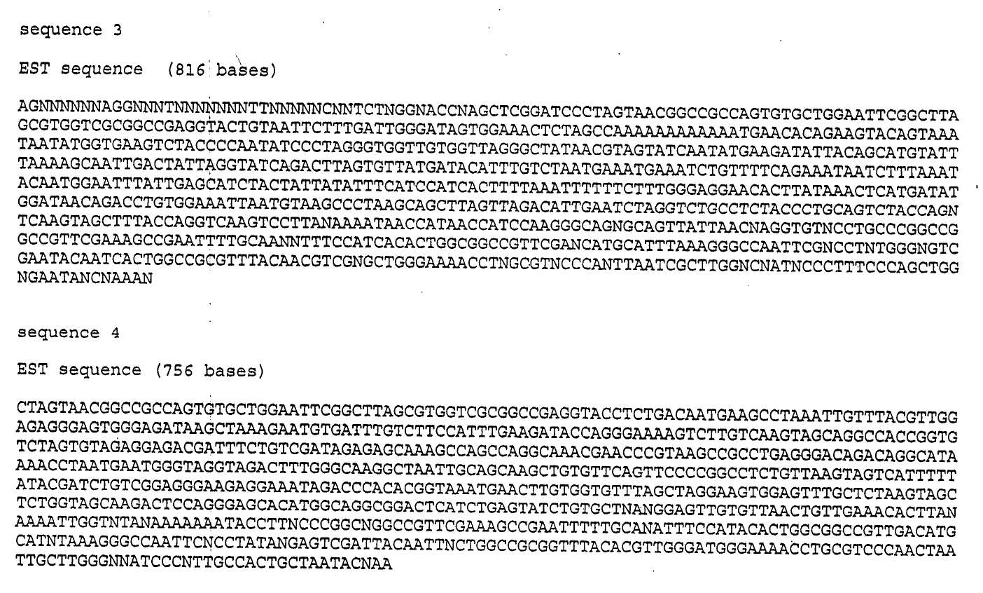

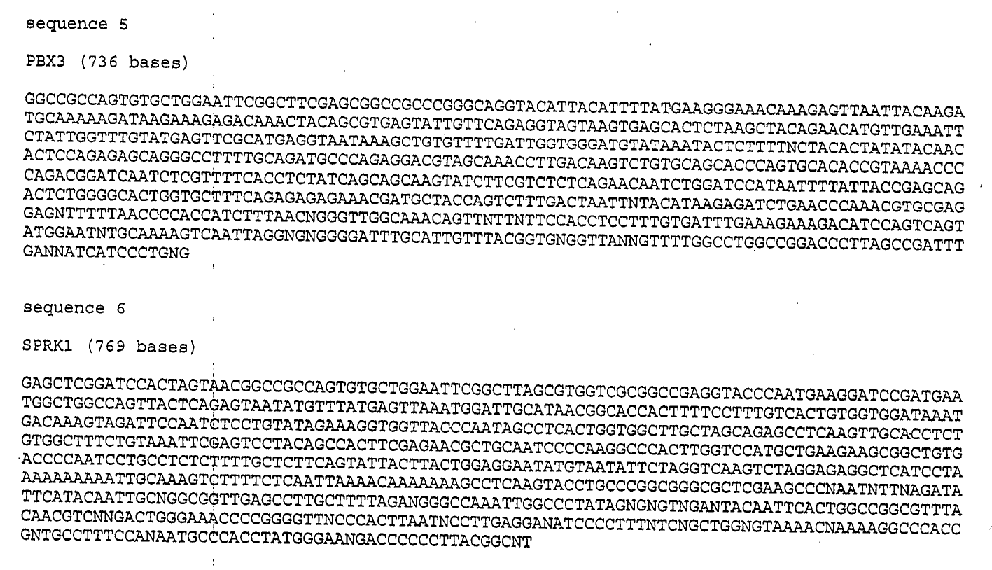

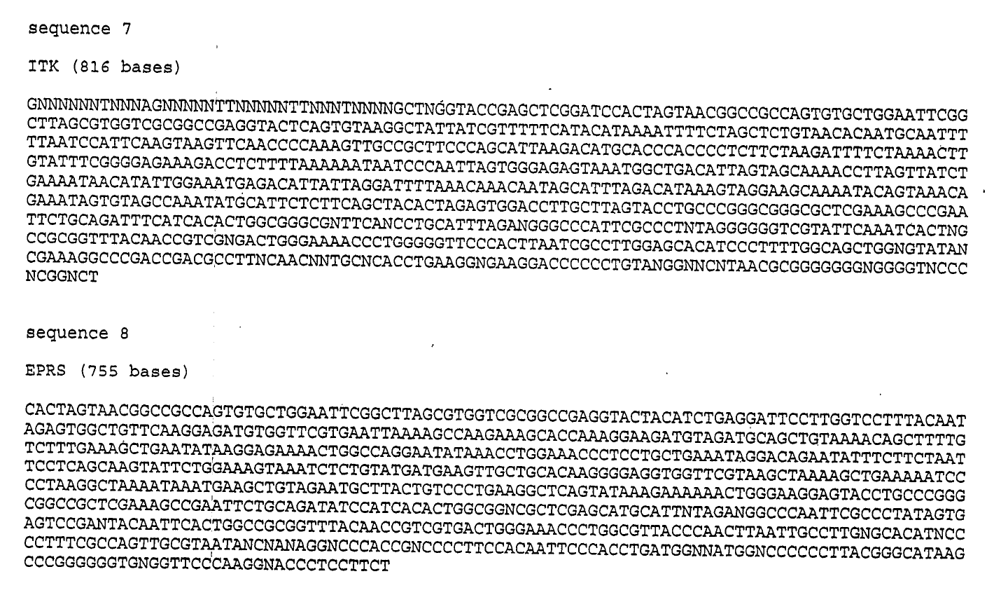

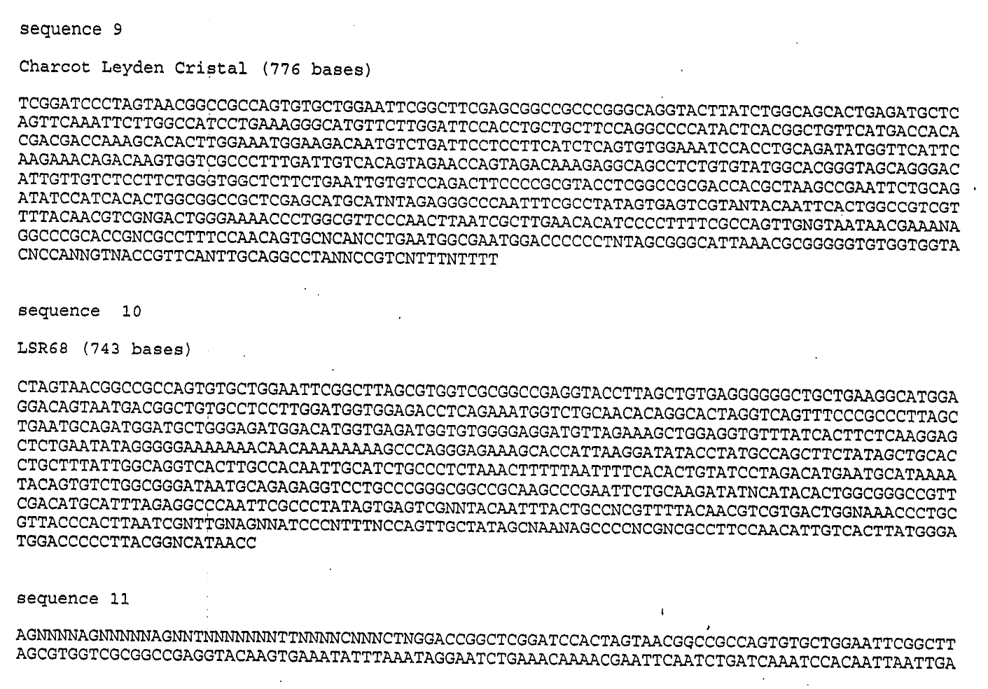

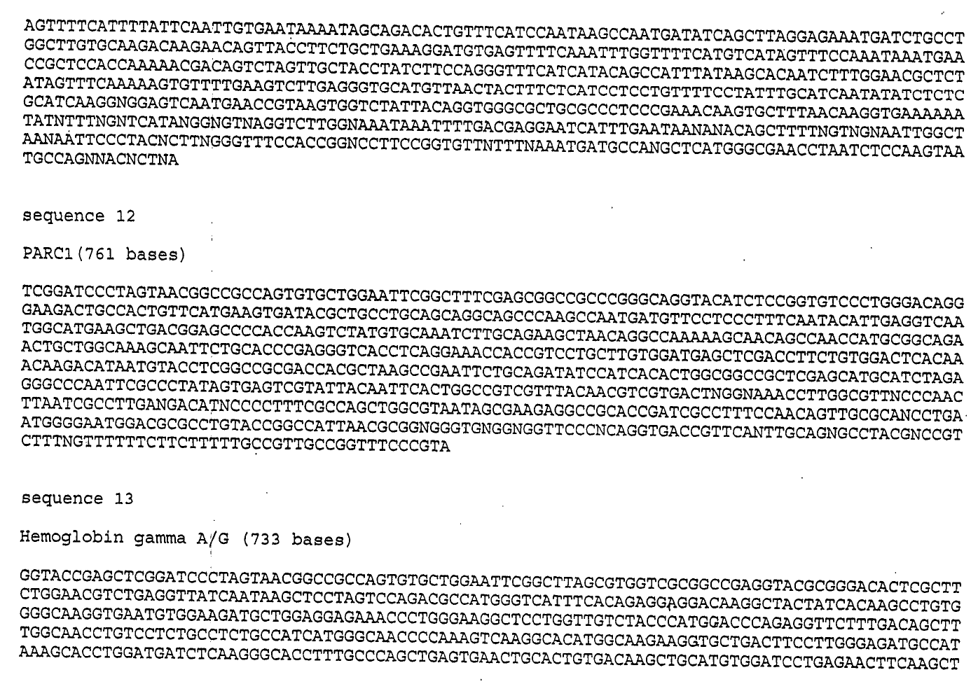

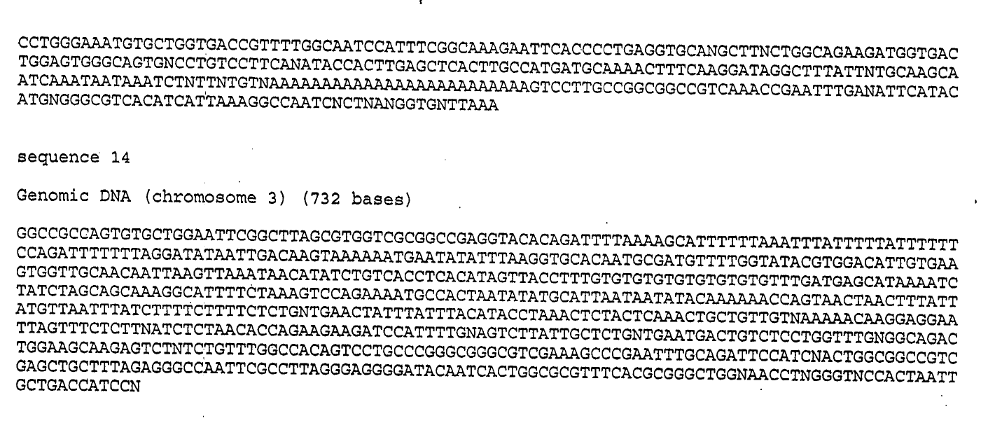

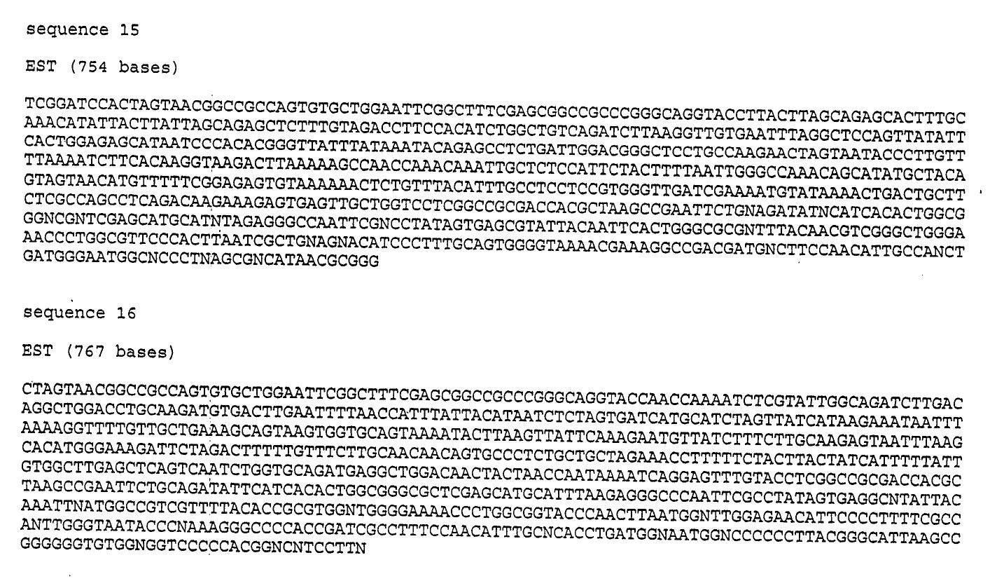

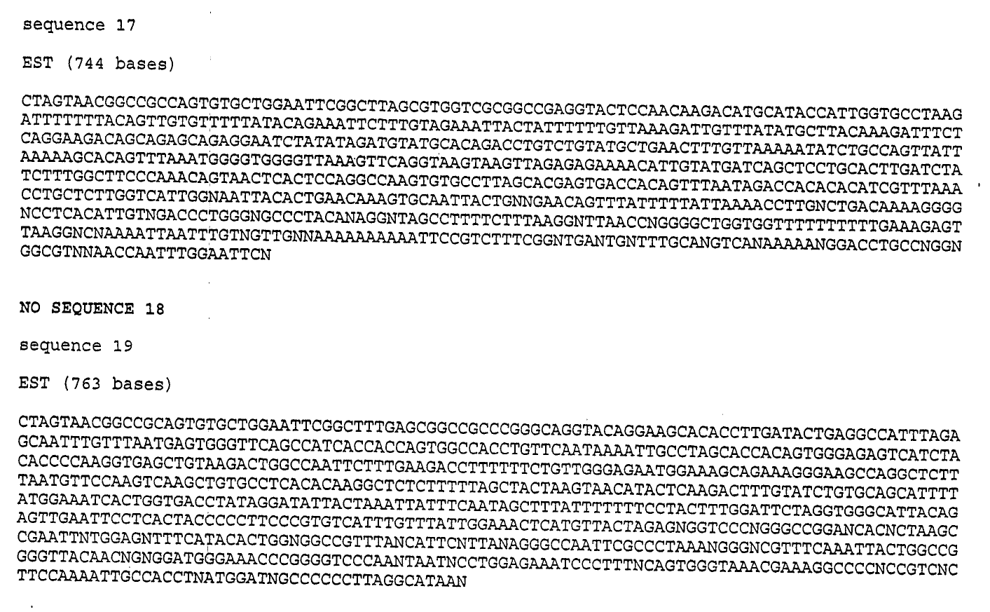

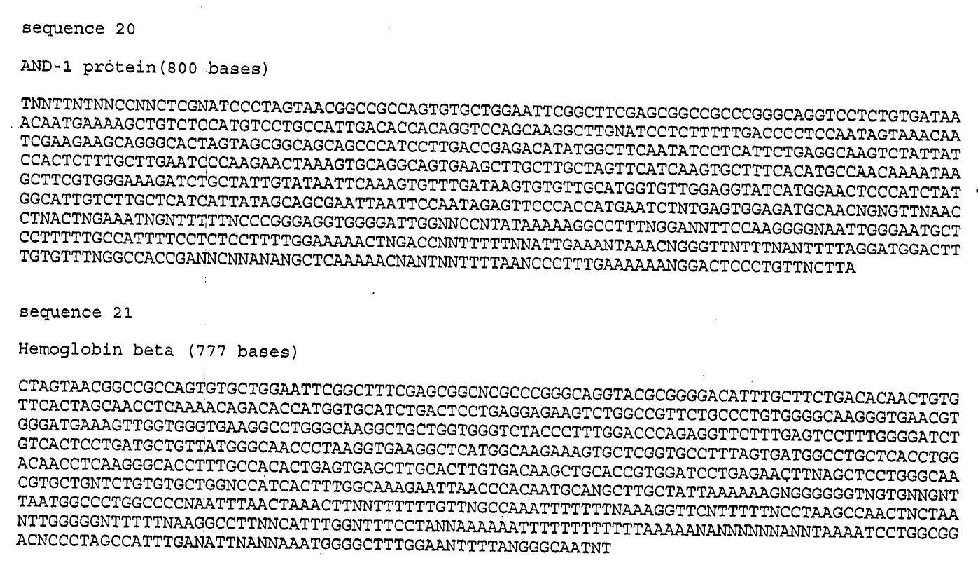

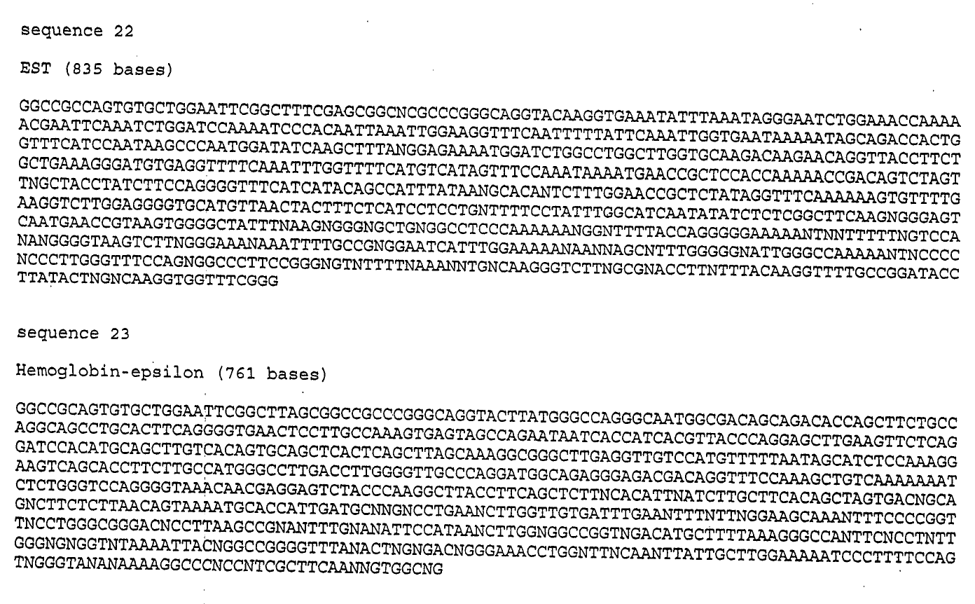

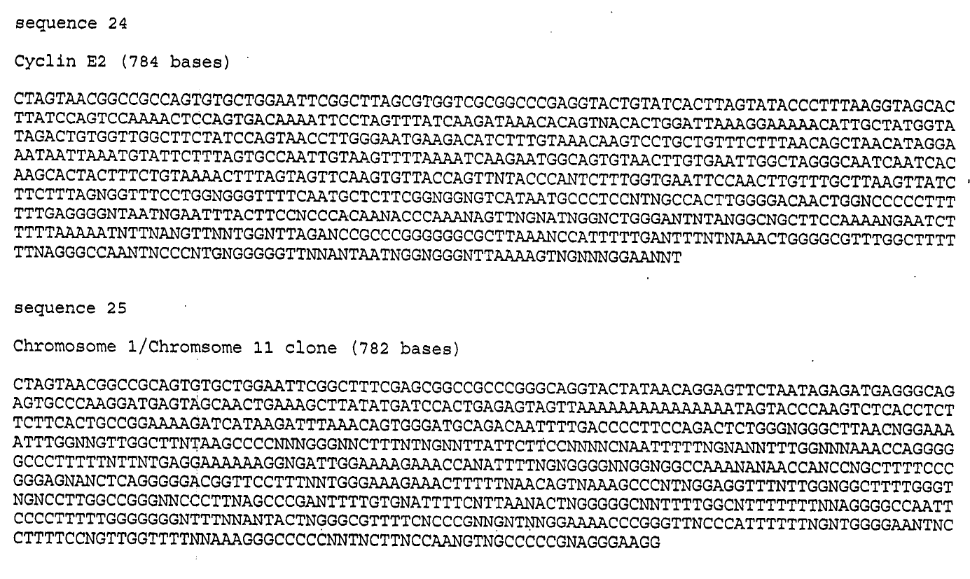

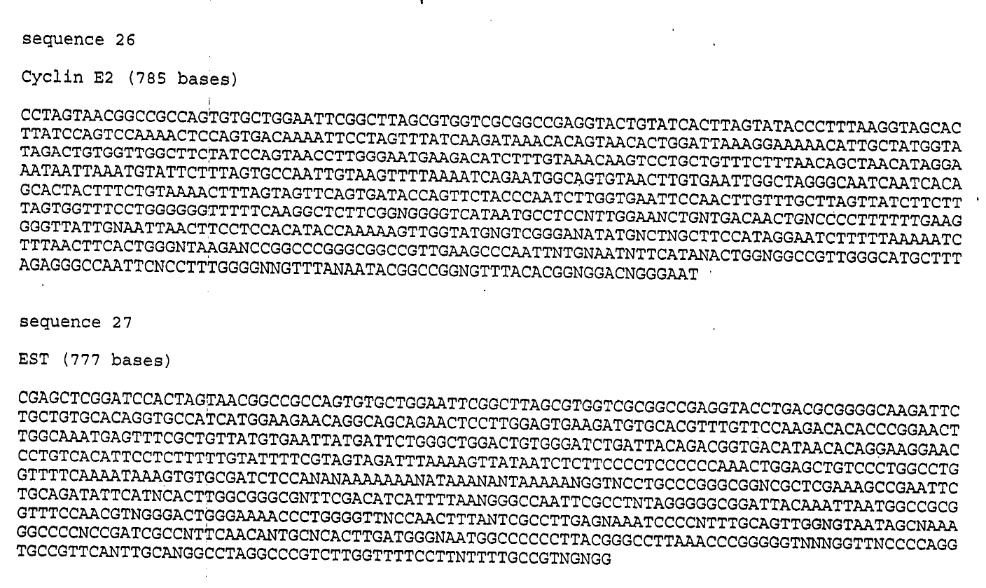

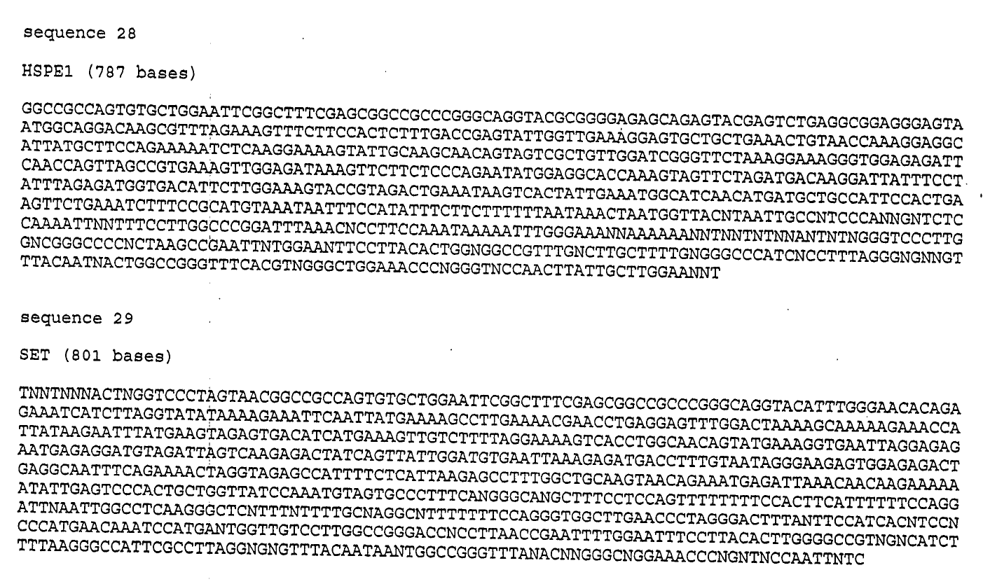

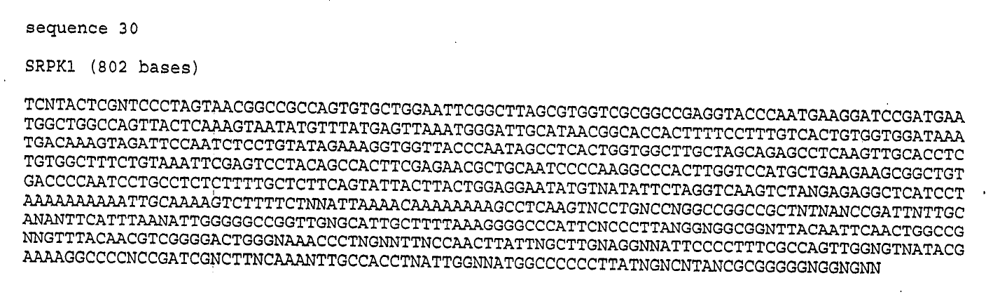

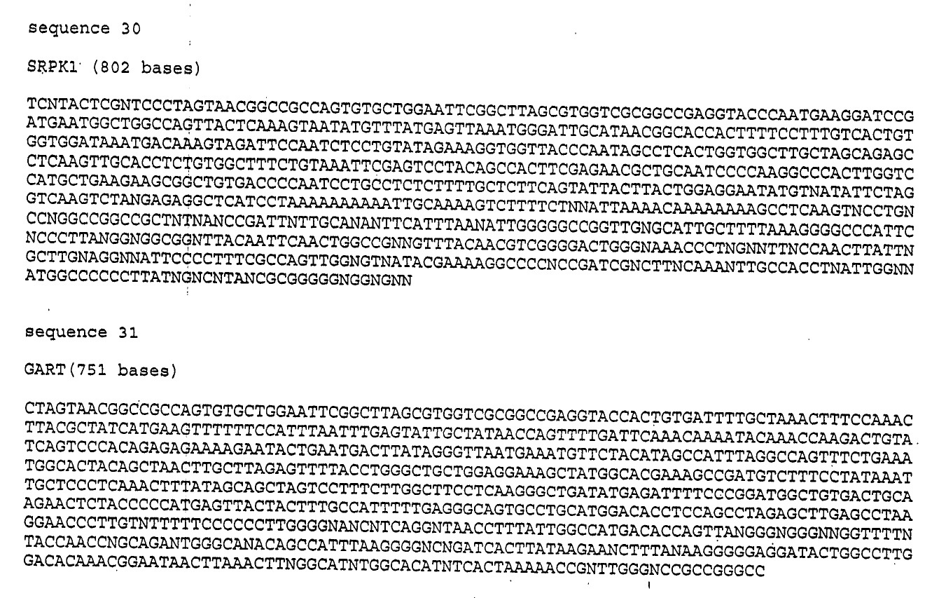

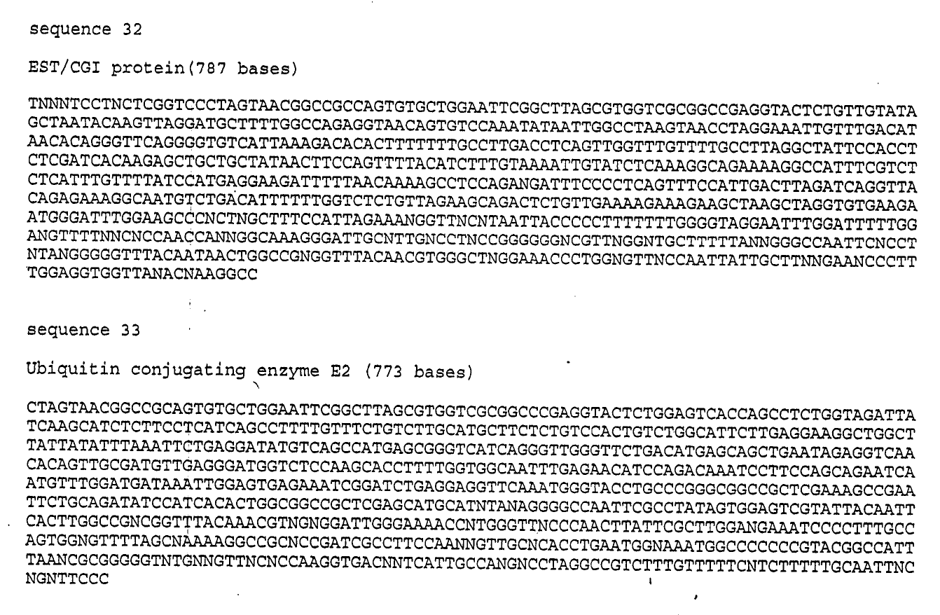

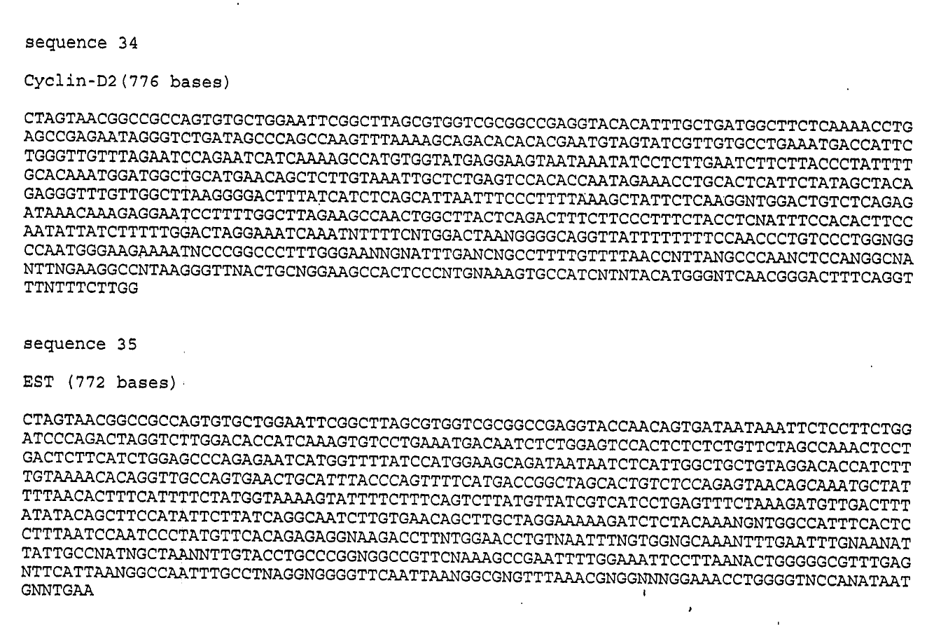

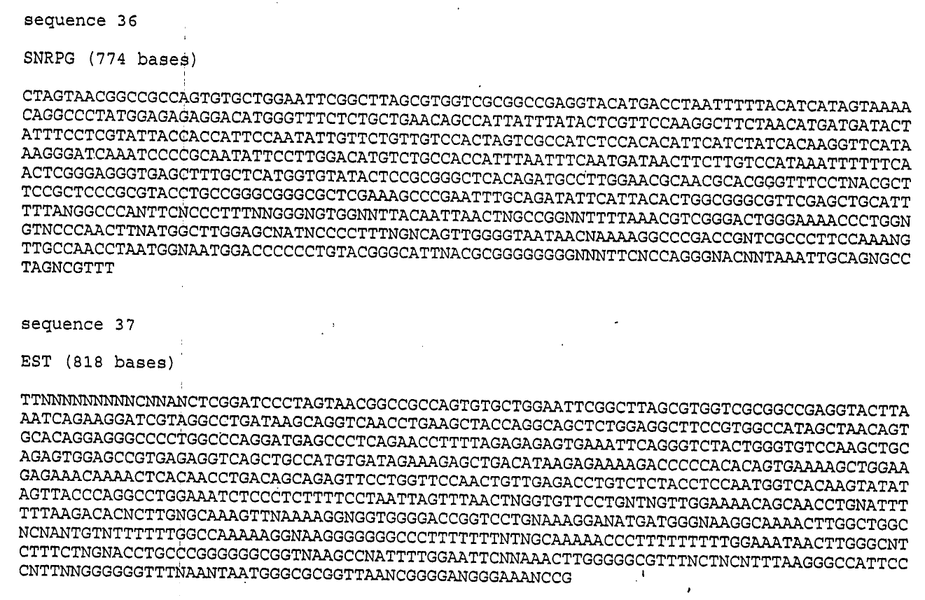

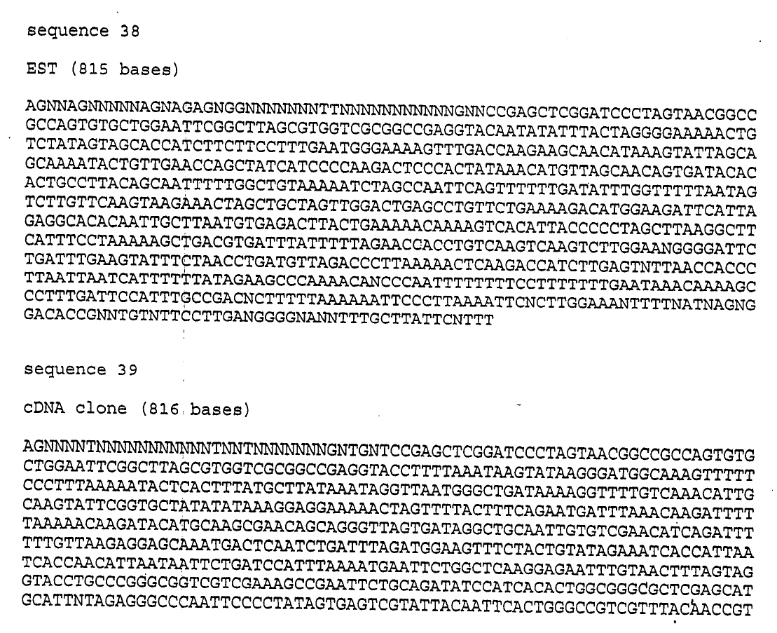

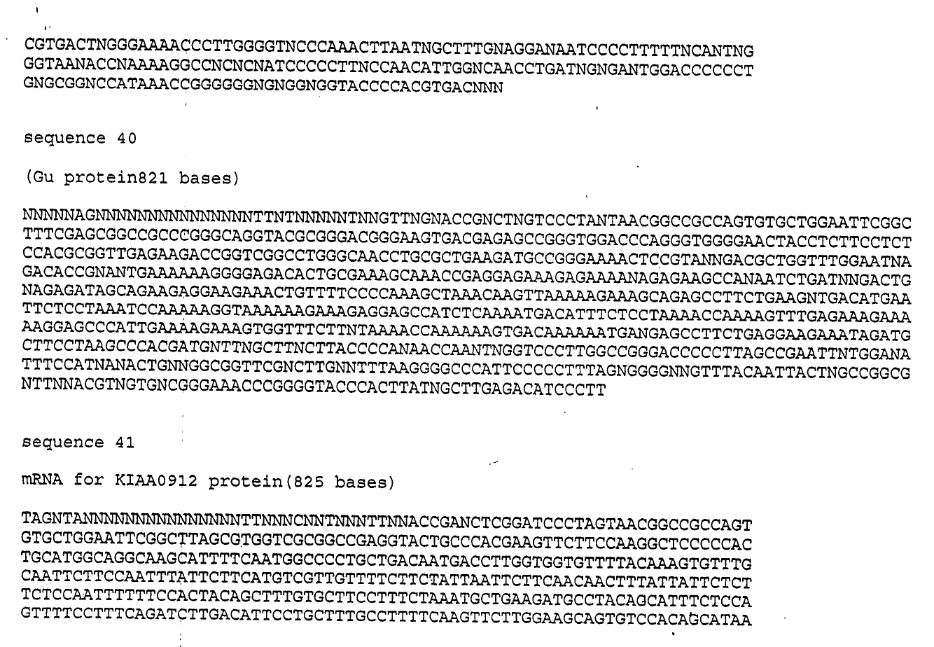

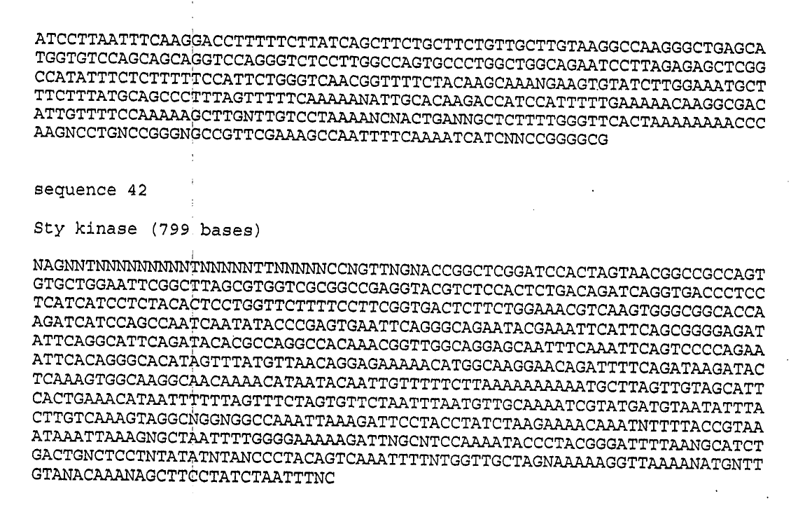

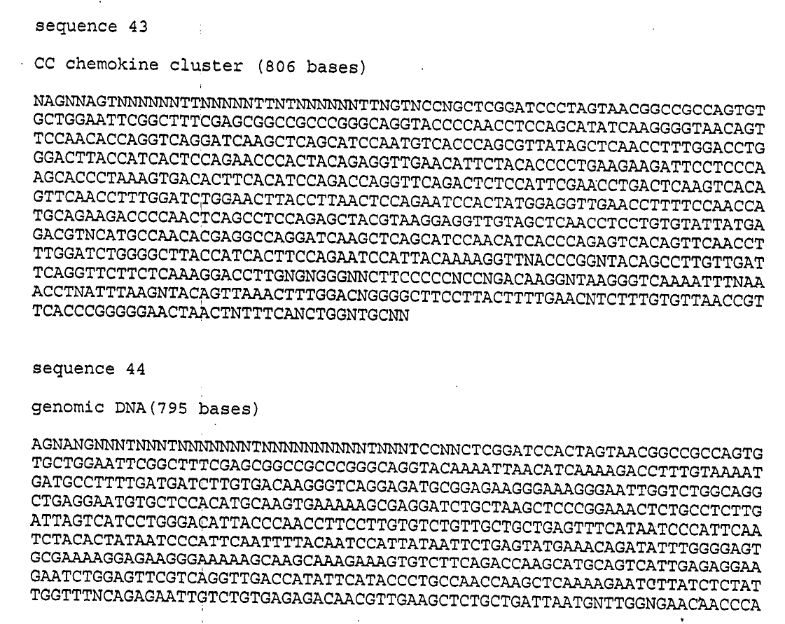

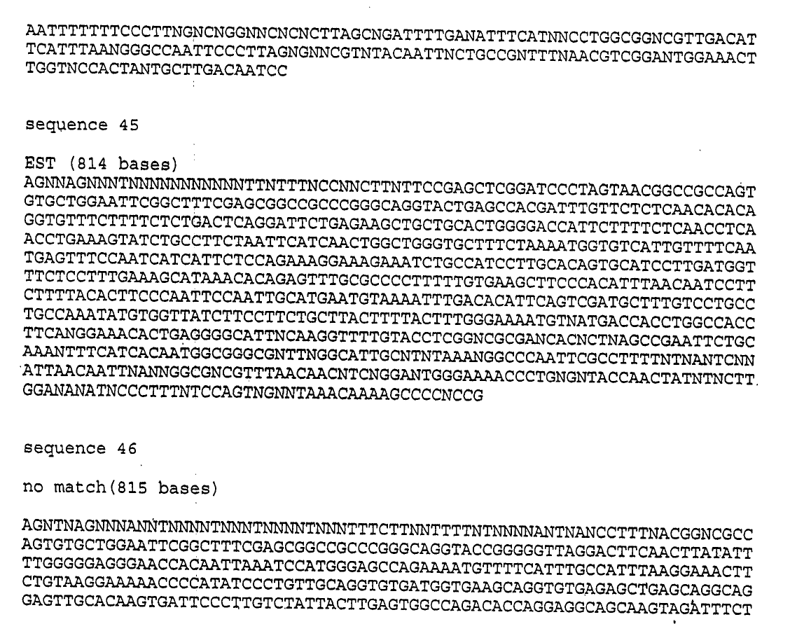

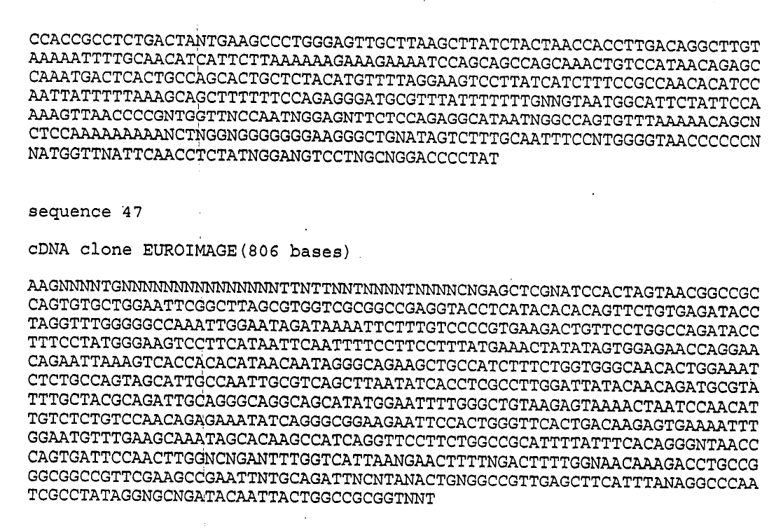

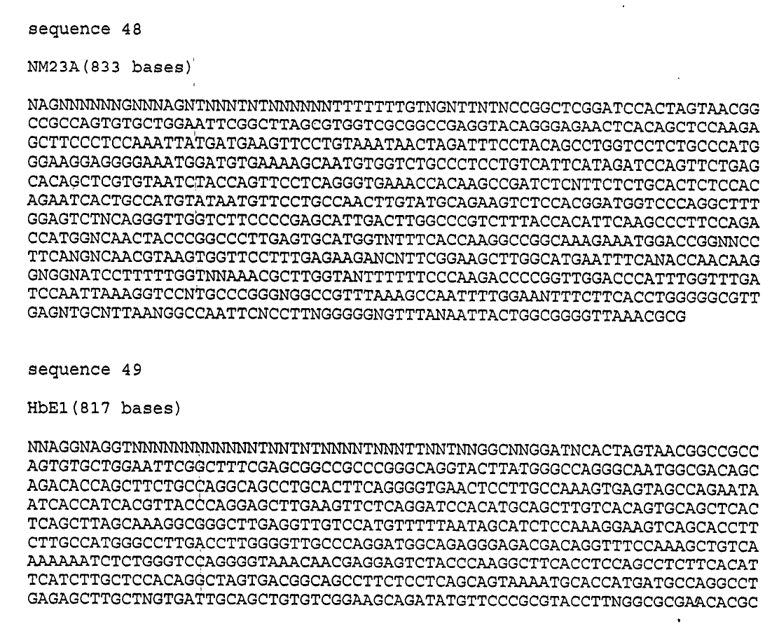

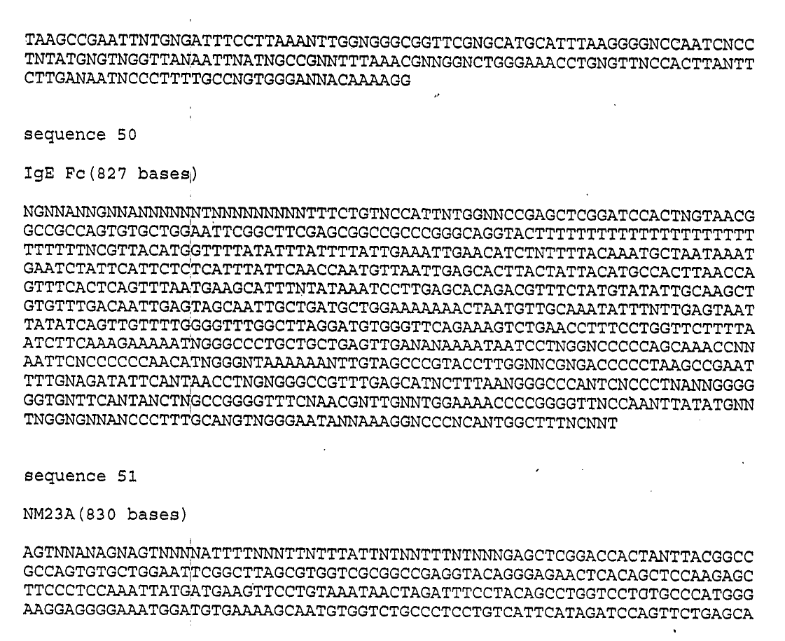

### DETAILED DESCRIPTION OF THE INVENTION

Definitions

“Marker genes” are genes that impart a distinct phenotype to cells expressing that gene and thus allow such cells to be distinguished from cells that do not have the marker gene. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a “reporter” trait that one can identify through observation or testing, i.e., by ‘screening’. Of course, many examples of suitable marker genes are known to the art and can be employed in the practice of the invention. Screenable markers that may be employed include, but are not limited to, a β-glucuronidase or uidA gene (GUS), a β-lactamase gene, a β-galactosidase gene, a luciferase (luc) gene, an aequorin gene, a green fluorescent protein gene (GFP) gene, a blue, red or yellow fluorescent protein gene, a chloramphenicol acetyltransferase gene (CAT), horseradish peroxidase gene (HRP), an alkaline phosphatase gene (AP) and others. Thus, a “marker gene” is one which is detectable by spectroscopic, photochemical, biochemical, immunochemical, or chemical means. Preferred marker genes are those which are detectable without disruption of a cell, including genes that confer resistance to a chemical or drug.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like.

Similarly, enzymatic labels may be detected by providing appropriate substrates for the enzyme and detecting the resulting reaction product. Finally, simple colorimetric labels are often detected simply by observing the color associated with the label.

As used herein, the terms “isolated and/or purified” refer to ill vitro isolation of a RNA, DNA or polypeptide molecule from its natural cellular environment, and from association with other components of the cell, such as nucleic acid or polypeptide, so that is can be sequenced, replicated and/or expressed. For example, “an isolated nucleic acid molecule” of the invention is RNA or DNA containing greater than 7, preferably 15, and more preferably 20 or more sequential nucleotide bases that hybridize to the RNA or DNA corresponding to any one of SEQ ID Nos. 1-79, or the complement thereof, and remain stably bound under moderate or stringent conditions, as defined by methods well known to the art, e.g., in Sambrook et al. (1989).

A nucleic acid molecule which “hybridizes” to a reference nucleic acid sequence duplexes or binds to that nucleic acid. A nucleic acid molecule which “hybridizes” to a reference sequence can include sequences which are shorter or longer than the reference sequence. Typically, the DNA:DNA hybridization is done in a Southern blot protocol using a 0.2×SSC, 0.1% SDS, 65° C. wash. The term “SSC” refers to a citrate-saline solution of 0.15 M sodium chloride and 20 mM sodium citrate. Solutions are often expressed as multiples or fractions of this concentration. For example, 6×SSC refers to a solution having a sodium chloride and sodium citrate concentration of 6 times this amount or 0.9 M sodium chloride and 120 mM sodium citrate. 0.2×SSC refers to a solution 0.2 times the SSC concentration or 0.03 M sodium chloride and 4 mM sodium citrate. Accepted means for conducting hybridization assays are known and general overviews of the technology can be had from a review of: Hames and Higgins, 1985; Meinkoth and Wahl, 1984; Sambrook et al., 1989; and Innis et al., 1990.

“Moderate” and “stringent” hybridization conditions are well known to the art, see, for example sections 9.47-9.51 of Sambrook et al. (1989). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS.

“Denaturation” refers to the process by which a double-stranded nucleic acid is converted into its constituent single strands. Denaturation can be achieved, for example, by the use of high temperature, low ionic strength, acidic or alkaline pH, and/or certain organic solvents. Methods for denaturing nucleic acids are well-known in the art.

“Annealing” or “hybridization” refers to the process by which complementary single-stranded nucleic acids form a double-stranded structure, or duplex, mediated by hydrogen-bonding between complementary bases in the two strands. Annealing conditions are those values of, for example, temperature, ionic strength, pH and solvent which will allow annealing to occur. Many different combinations of the above-mentioned variables will be conducive to annealing. Appropriate conditions for annealing are well-known in the art, and will generally include an ionic strength of 50 mM or higher monovalent and/or divalent cation at neutral or near-neutral pH. An annealing mixture is a composition containing single-stranded nucleic acid at the appropriate temperature, pH and ionic strength to allow annealing to occur between molecules sharing regions of complementary sequence.

A “duplex” refers to a double-stranded polynucleotide.

“Amplification” is the process by which additional copies of a nucleic acid sequence or collection of nucleic acid sequences are generated. Amplification is generally achieved enzymatically, using a DNA polymerase enzyme. Current techniques allow exponential amplification of any sequence flanked by binding sites for a pair of oligonucleotide primers, through reiterative application of denaturation, primer annealing and polymerase extension steps, commonly known as a polymerase chain reaction. U.S. Pat. No. 4,683,202, Saiki et al., 1988; Innis et al., 1990; Ehrlich, 1989. Under the most widely-practiced conditions of the polymerase chain reaction, the rate of polymerization is approximately 1,000-2,000 nucleotides per minute. Accordingly, the maximum length of amplifiable sequence will be limited by the reaction conditions (for example, the duration of the extension step). The ability to control the extent of elongation in a polymerase chain reaction can be used to advantage to generate lower-complexity subsets of amplified fragments from an initial fragment collection of high complexity.

Nucleic acid molecules of interest in the present invention may be cloned or amplified, e.g., by in vitro methods, such as the polymerase chain reaction (PCR), the ligase chain reaction (LCR), the transcription-based amplification system (TAS), the self-sustained sequence replication system (3SR) and the Qβ replicase amplification system (QB). A wide variety of cloning and in vitro amplification methodologies are well-known to persons of skill. Examples of these techniques and instructions sufficient to direct persons of skill through many cloning exercises are found in Berger and Kimmel; Sambrook et al., 1989; Ausubel et al., 1994; U.S. Pat. No. 5,017,478; and European Patent No. 0,246,864.

A “polynucleotide”, “nucleic acid”, “nucleic acid molecule”, “nucleic acid sequence” or “nucleic acid segment” is a polymer of nucleotides, and the terms are meant to encompass both RNA and DNA, as well as single-stranded and double-stranded polynucleotides, as well as molecules containing modifications of the base, sugar or phosphate groups as are known in the art.

A “population” of polynucleotides, or nucleic acid molecules, sequences or segment is any collection comprised of different nucleotide sequences. Examples of polynucleotide populations include, but are not limited to, those which represent the genome of a normal cell, the genome of an infected cell, the genome of a neoplastic cell, the genome of a cell existing in a pathological state, the DNA that is characteristic of a particular cell, multicellular structure, organism, state of differentiation, pathological or non-pathological state, the total RNA population of a cell, the polyadenylated RNA population of a cell, or a cDNA population representative of the mRNA population of a particular cell, multicellular structure, organism, state of differentiation, pathological or non-pathological state. Using the methods of the invention, a sample population of polynucleotides comprising more than one different polynucleotide sequence is compared to a sample from a control population of polynucleotides to identify nucleic acid molecules that are unique to the sample population.

A “primer” is an oligonucleotide capable of base-pairing with a polynucleotide and serving as a site from which polymerization can be initiated.

An “oligonucleotide” is a short nucleic acid, generally DNA and generally single-stranded. Generally, an oligonucleotide will be shorter than 200 nucleotides, more particularly, shorter than 100 nucleotides, even more particularly, 50 nucleotides or shorter, but greater than 7 nucleotides, and preferably greater than 10 nucleotides, in length.

“Genomic” DNA is DNA obtained from a cell representing all or part of the genome of that cell.

cDNA or “complementary DNA” is DNA obtained from copying RNA by reverse transcription. It most often represents the population of mRNA molecules found in a particular cell, cell type, state of development or pathological state.

A “variant” polypeptide of the invention has at least about 80%, more preferably at least about 90%, and even more preferably at least about 95%, but less than 100%, contiguous amino acid sequence identity to a polypeptide having an amino acid sequence encoded by an open reading frame comprising any one of SEQ ID NOs:1-79, or a fragment thereof. A preferred variant polypeptide includes a variant polypeptide or fragment thereof having at least about 1%, more preferably at least about 10%, and even more preferably at least about 50%, the activity of the polypeptide having the amino acid sequence encoded by DNA comprising any one of SEQ ID NOs:1-79.

A “variant” nucleic acid sequence of the invention has at least about 80%, more preferably at least about 90%, and even more preferably at least about 95%, but less than 100%, contiguous nucleic acid sequence identity to a nucleic acid sequence comprising any one of SEQ ID NOs:1-79, or a fragment thereof. The amino acid and/or nucleic acid similarity (or homology) of two sequences may be determined manually or using algorithms well known to the art.

The term “sequence homology” or “sequence identity” means the proportion of base matches between two nucleic acid sequences or the proportion amino acid matches between two amino acid sequences. The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

Gaps (in either of the two sequences) are permitted to maximize matching; gap lengths of 15 bases or less are usually used, 6 bases or less are preferred with 2 bases or less more preferred. When using oligonucleotides as probes; the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. Preferably, default parameters are employed.

As applied to polypeptides, the term “substantial identity” means that two peptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

Nucleic Acid Molecules of the Invention and the Polypeptides Encoded Thereby

The ability to identify nucleic acid sequences which are expressed in one nucleic acid sample and not in another is of intense interest in the field of molecular biology. The identification of differentially expressed nucleic acid sequences can provide valuable clues as to genetic bases for disease, inherited dominant and recessive traits, genetic alterations which give rise to diseases such as cancer, determining species similarities and differences, genotyping, and taxonomic classification.

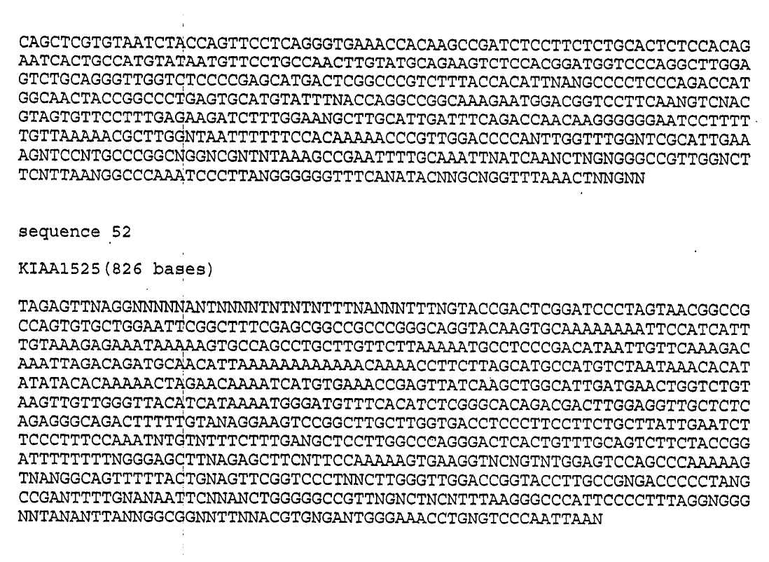

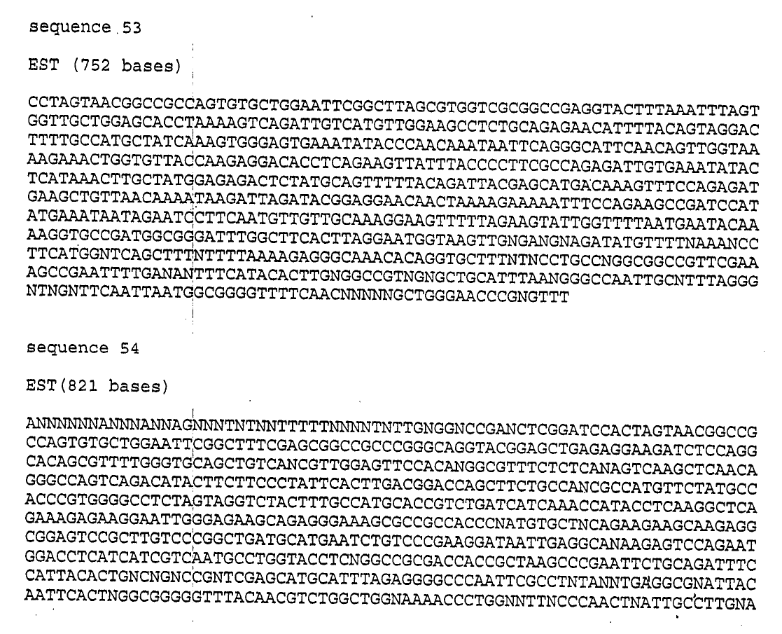

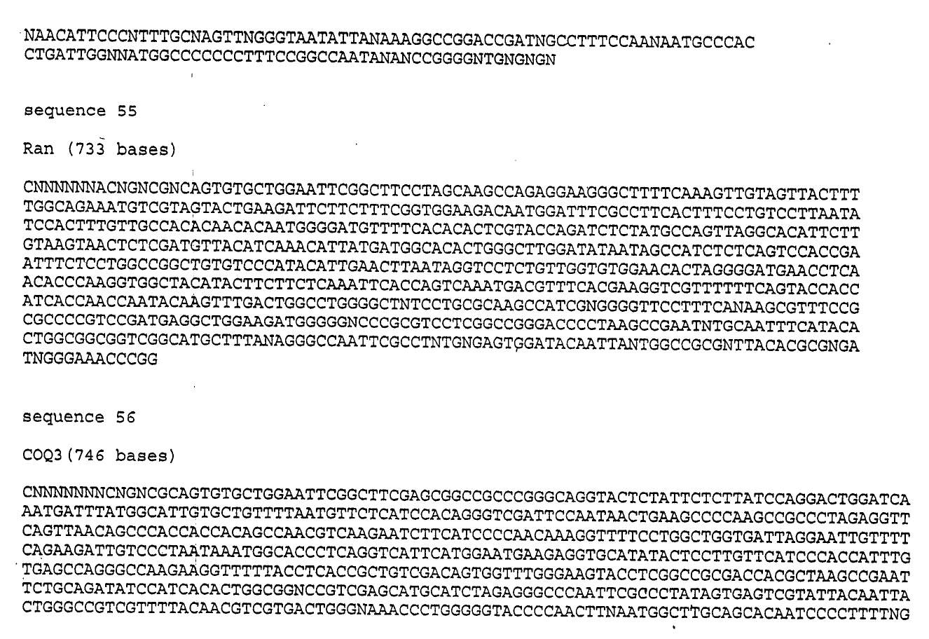

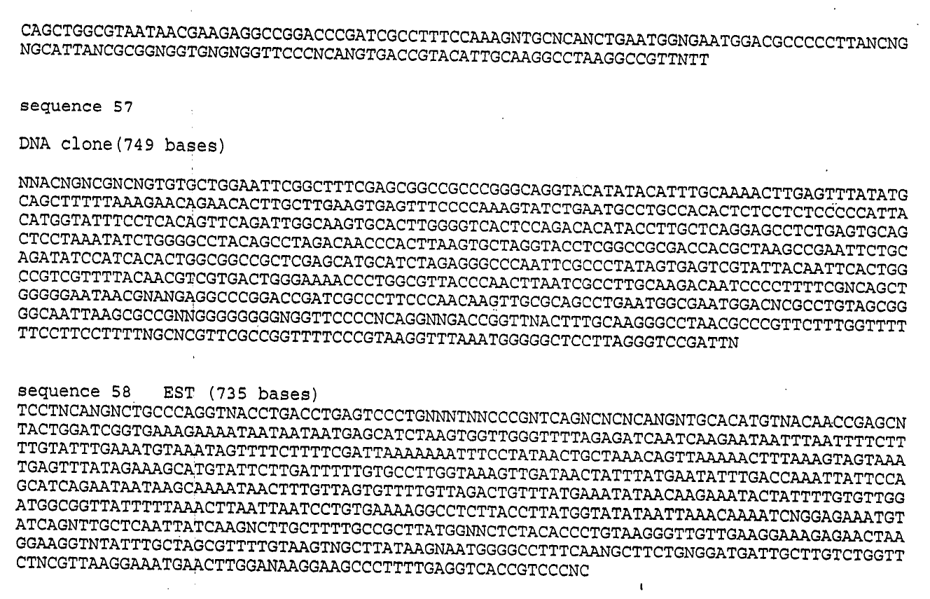

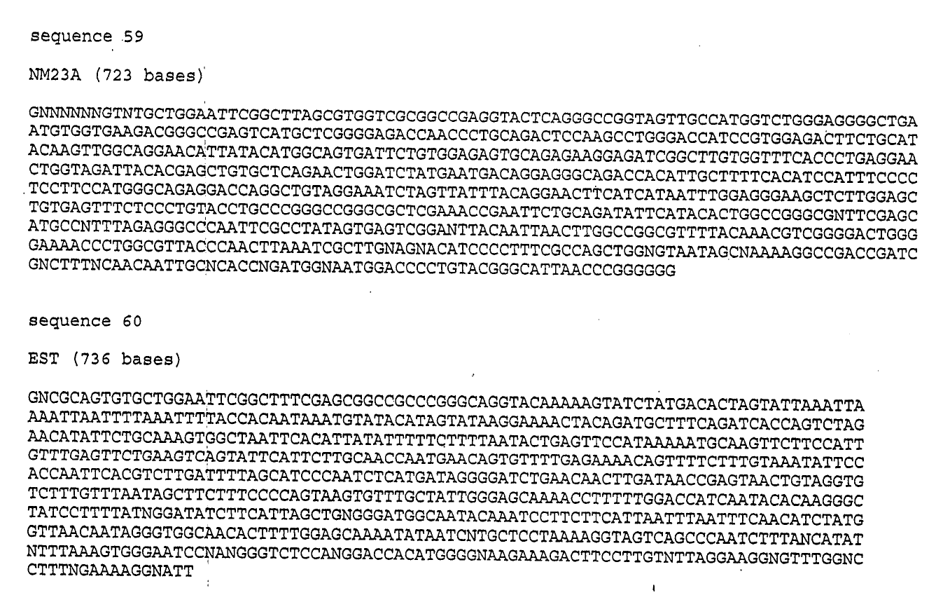

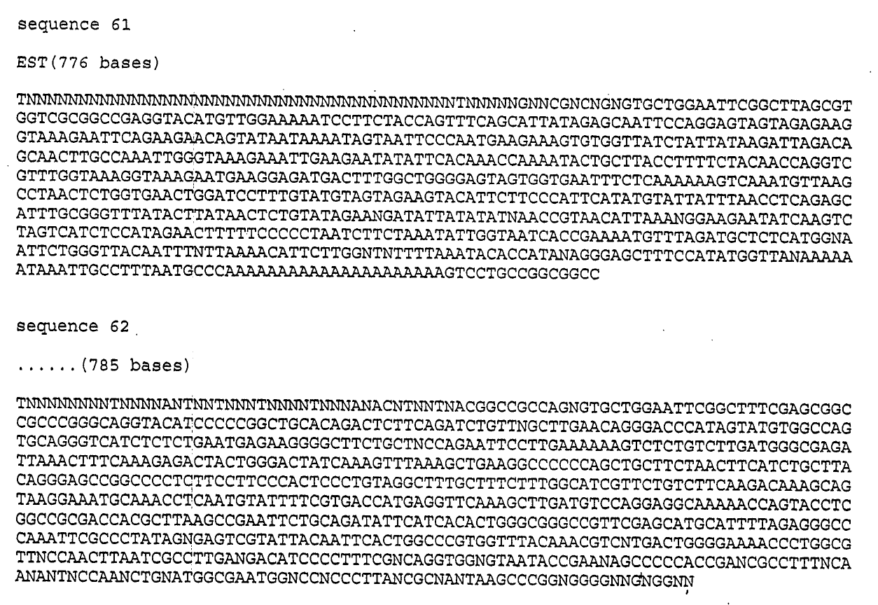

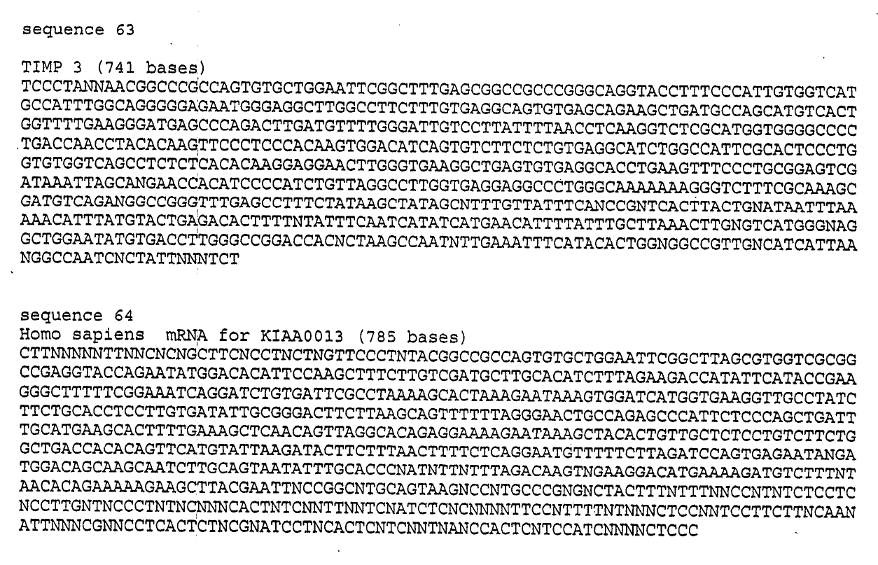

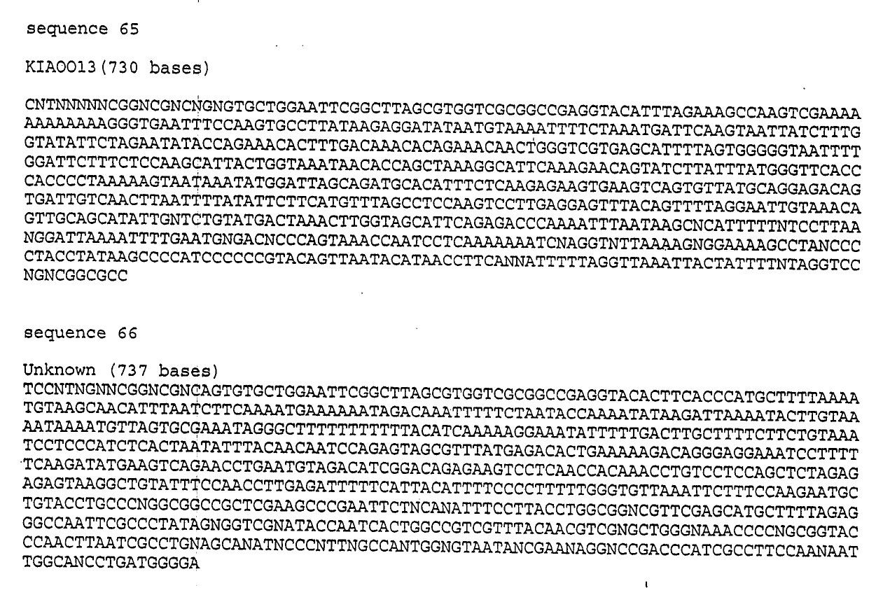

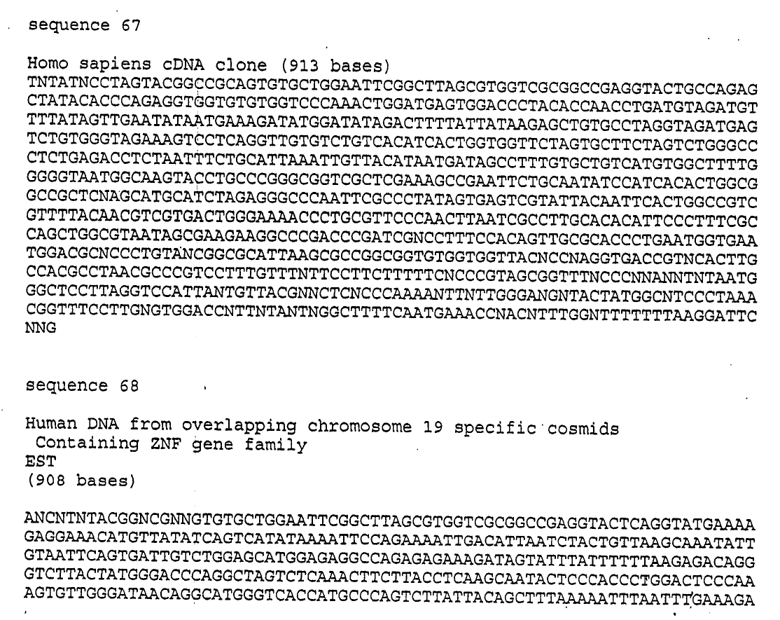

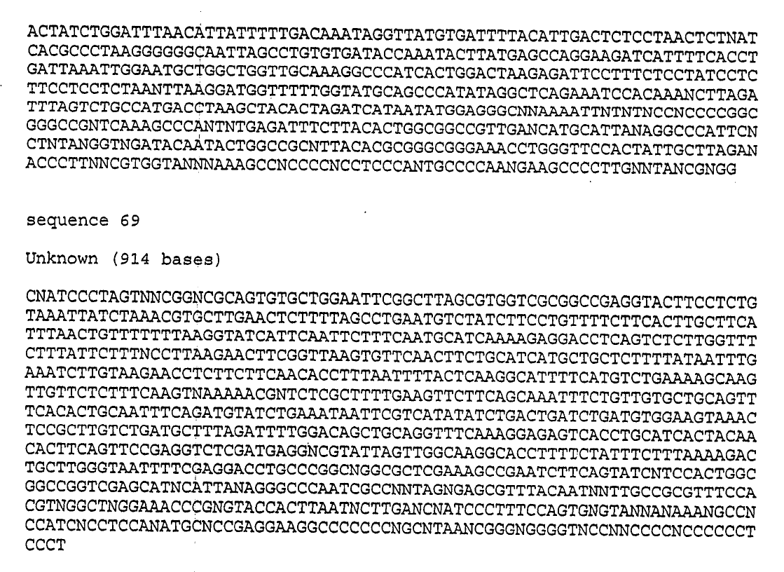

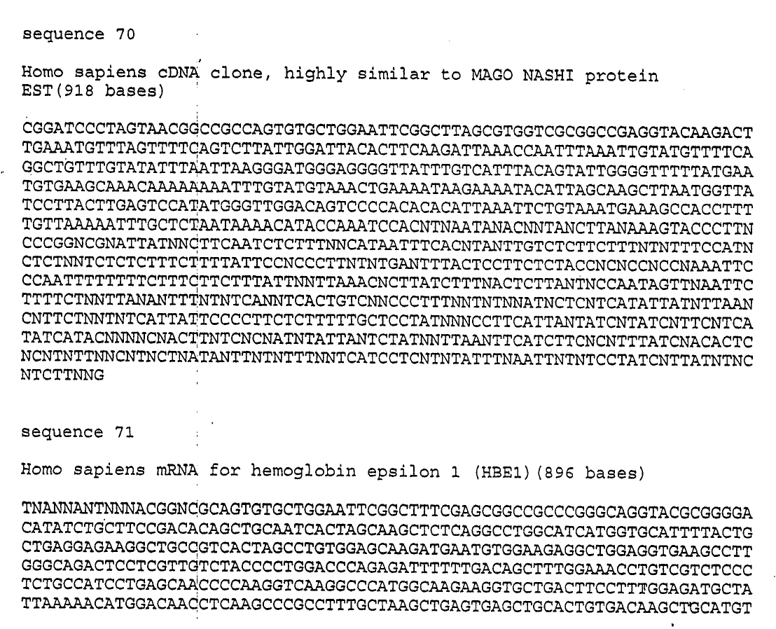

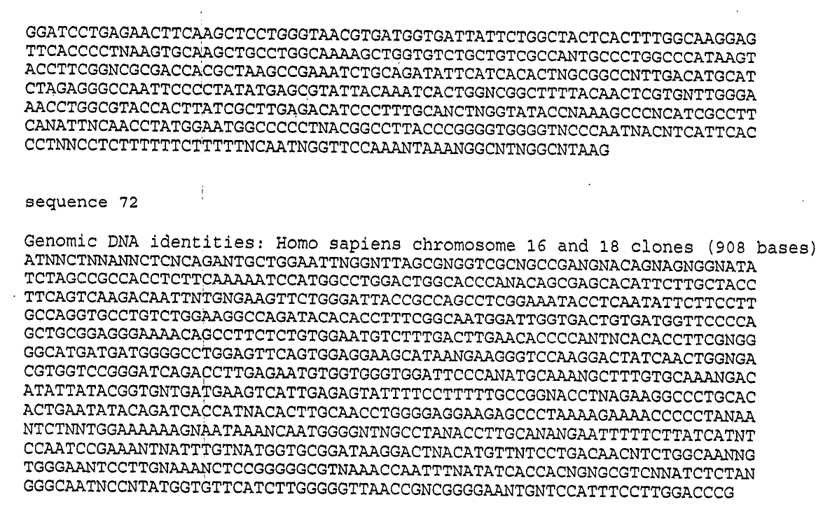

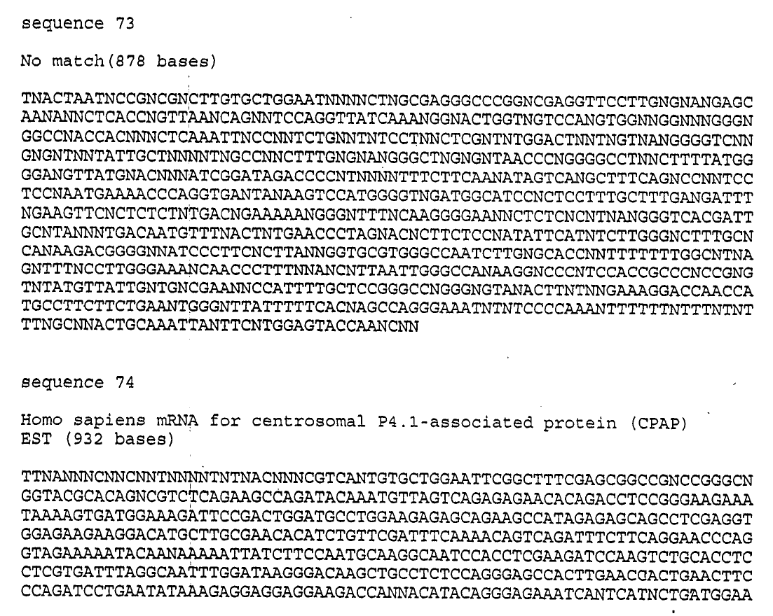

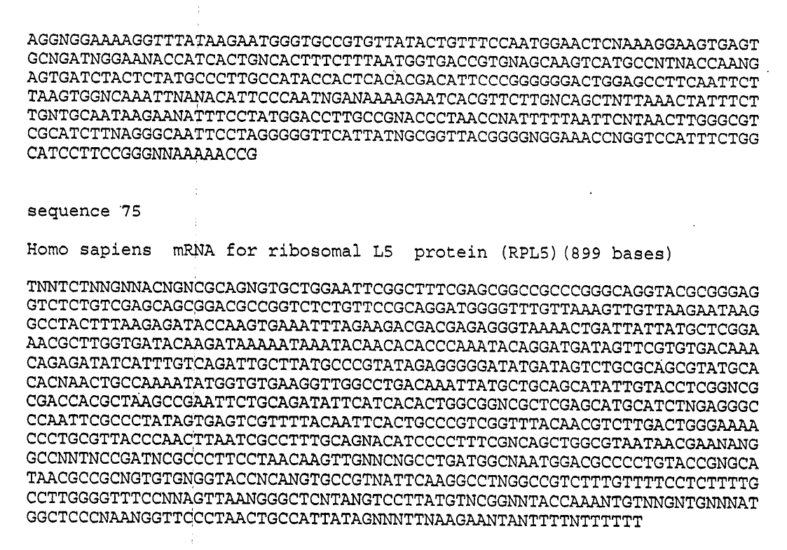

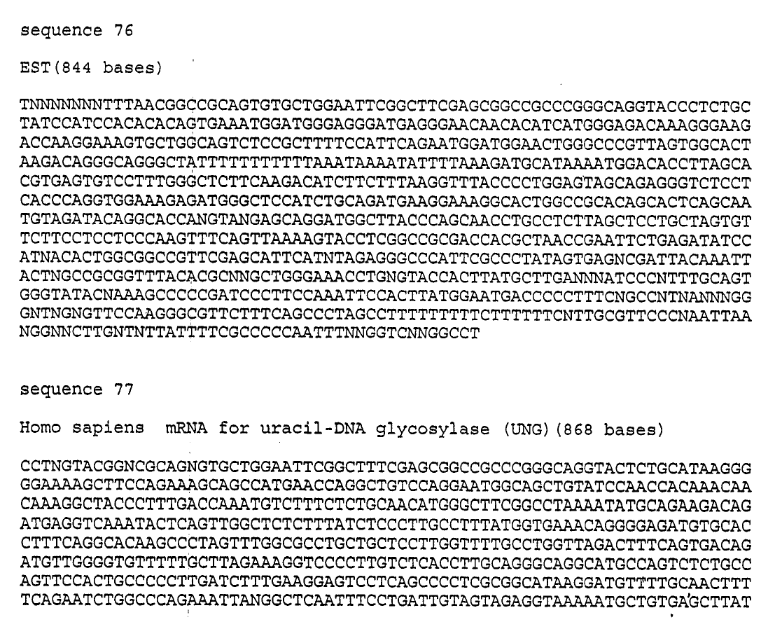

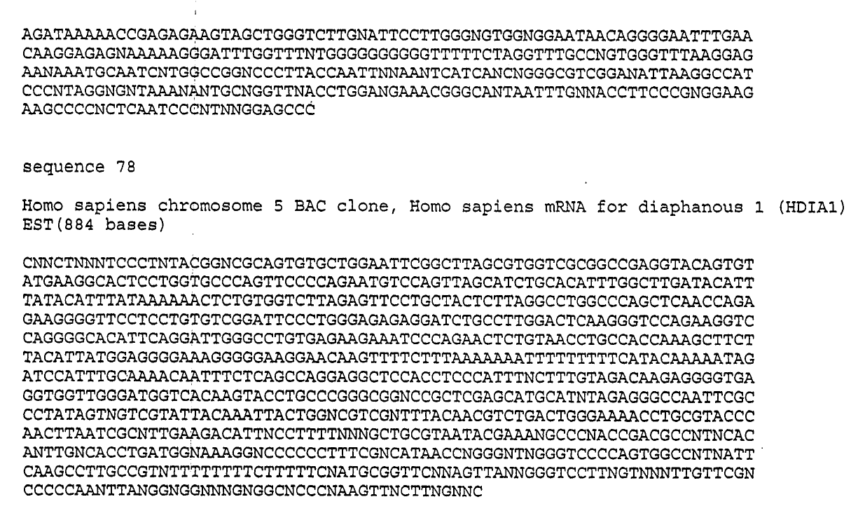

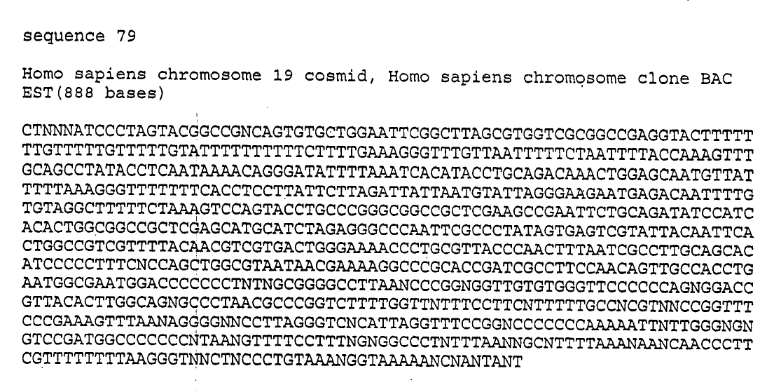

The present invention relates to isolated nucleic acid molecules that are specifically or differentially expressed in cells which express a protein associated with disease. Thus, the expression of the nucleic acid molecules of the invention is induced by the protein associated with disease or are otherwise downstream of the expression of the disease-associated protein in a pathway that links the two. Exemplary nucleic acid molecules which are expressed in p210^{BCR-ABL}-expressing cells are described in FIG. 3. These sequences are useful as nucleic acid probes for hybridization assays and as primers, e.g., for use in amplification reactions. The present invention also relates to isolated polypeptides, as well as to methods for obtaining isolated polypeptides, e.g., by producing recombinant polypeptides, encoded by the nucleic acid molecules of the invention. The present invention also relates to antibodies which specifically bind to the encoded gene products, and methods of therapy for diseases, e.g., for ALL and CML and other types of cancer.

In one embodiment of the invention, over 50 different cDNAs were isolated and sequenced, the RNA of which is expressed in p210^{BCR-ABL} expressing cells. Exemplary nucleic acid molecules include RNA or DNA corresponding to any one of SEQ ID NOs:1-79, the complement thereof, or a variant thereof. Thus, this embodiment of the invention includes nucleic acid molecules comprising: a) the nucleotide sequence of any one of SEQ ID NOs:1-79 or a portion thereof; b) DNA, genomic or cDNA, comprising an open reading frame comprising any one of SEQ ID NOs:1-79, or the complement thereof, e.g., one which encodes a fill length polypeptide; or c) DNA which hybridizes to any one of SEQ ID NOs:1-79, the complement thereof, or a portion thereof, e.g., under moderate hybridization conditions. The nucleic acid molecules can be obtained from sources in which it occurs in nature (e.g., tissue or cell samples), from a DNA library, by means of recombinant technology or amplification procedures, or by synthetic techniques. Preferably, the nucleic acid source is mammalian, preferably primate, and more preferably human, DNA.

The nucleic acid molecules of the present invention can be antisense nucleic acid molecules. Antisense nucleic acid is complementary, in whole or in part, to a sense strand and can hybridize with the sense strand. The target can be DNA, or its RNA counterpart (i.e., wherein T residues of the DNA are U residues in the RNA counterpart). When introduced into a cell, antisense nucleic acid can inhibit the expression of the gene encoded by the sense strand. Antisense nucleic acids can be produced by standard techniques. In a particular embodiment, the nucleic acid molecule is wholly or partially complementary to and hybridizes with a nucleic acid having a sequence comprising any one of SEQ ID NOs:1-79.

Nucleic acids referred to herein as “isolated” are nucleic acids separated away from the nucleic acids of the genomic DNA or cellular RNA of their source of origin (e.g., as it exists in cells or in a mixture of nucleic acids such as a library), and may have undergone further processing. “Isolated” nucleic acids include nucleic acids obtained by methods described herein, similar methods or other suitable methods, including essentially pure nucleic acids, nucleic acids produced by chemical synthesis, by combinations of biological and chemical methods, and recombinant nucleic acids which are isolated.

Nucleic acids referred to as “recombinant” are nucleic acids which have been produced by recombinant DNA methodology, including those nucleic acids that are generated by procedures which rely upon a method of artificial recombination, such as PCR and/or cloning into a vector using restriction enzymes.

A nucleic acid molecule of the invention can be operably linked to one or more expression control elements (an expression cassette) which is incorporated into a vector, e.g., a viral vector, and the resulting construct introduced into host cells, which are maintained under conditions suitable for expression of the encoded polypeptide. The construct can be introduced into cells by a method appropriate to the host cell selected (e.g., transformation, transfection, electroporation, or infection). The encoded polypeptide can be isolated from the host cells or medium.

The nucleic acid molecules can be also used as probes to detect and/or isolate (e.g., by hybridization with RNA or DNA) variants or homologs (i.e., in other species) thereof, and/or to identify sequences corresponding to full length open reading frames. Moreover, the presence or frequency of the RNA corresponding to a nucleic acid molecule of the invention may be indicative of a disease.

The nucleic acid molecules of the invention are also useful for therapeutic purposes. For example, sense or anti-sense DNA fragments can be introduced into expression cassette which are subsequently cells in which expression of a particular nucleic acid sequence is to be reduced. The sense or anti-sense DNA fragments are expressed so as to result in reduced expression of the corresponding gene.

Pharmaceutical compositions which comprise nucleic acid molecules of the present invention and a suitable carrier (e.g., a buffer) are also the subject of this invention. The compositions can include additional components, such as stabilizers.

The present invention also relates to polypeptides encoded by the nucleic acid molecules of the invention, including a variant polypeptide which has at least 80% or more contiguous amino acid sequence identity to the polypeptide encoded by an open reading frame comprising a nucleic acid molecule of the invention, and which variant polypeptide has at least 1%, preferably 10% or more, of the activity of the non-variant (wild type) polypeptide, which can be obtained (isolated) from sources (e.g., cells) in which they occur in nature, produced using recombinant or genetic engineering methods or synthesized chemically. It also relates to pharmaceutical compositions which comprise the polypeptide and an appropriate carrier, such as a buffer. It may also comprise other components, such as stabilizers and other drugs.

Polypeptides referred to herein as “isolated” are polypeptides purified to a state beyond that in which they exist in cells in which they are produced. “Isolated” polypeptides include polypeptides obtained by methods described herein, similar methods or other suitable methods, including essentially pure proteins or polypeptides isolated from the source in which they occur, polypeptides produced by chemical synthesis (e.g., synthetic peptides), or by combinations of biological and chemical methods, and recombinant polypeptides which are isolated.

Polypeptides referred to herein as “recombinant” or “recombinantly produced” are polypeptides produced by the expression of nucleic acids encoding the polypeptides in a host cell which is modified to contain the nucleic acids encoding the polypeptide (e.g., by transfection with exogenous DNA which encodes the polypeptide) or is modified to express a gene which induces the expression of the nucleic acid molecules of the invention.

Another aspect of the invention relates to a method of producing polypeptide encoded by a nucleic acid molecule of the invention, a variant, or a fragment thereof. Recombinant polypeptide can be obtained, for example, by the expression of a recombinant DNA molecule encoding the polypeptide, a variant or a fragment thereof in a suitable host cell. Alternatively, recombinantly produced polypeptide is expressed in a suitable host cell by turning on or enhancing expression of a gene corresponding to the nucleic acid molecule of the invention present in (endogenous to) the host cell. Constructs suitable for the expression of the polypeptide or a variant can be introduced into a suitable host cell. Cells which express a recombinantly produced polypeptide or variant thereof, can be maintained in culture. Such cells are useful for a variety of purposes and can be used in the production of polypeptide for characterization, isolation and/or purification (e.g., affinity purification), and as immunogens, for instance. Suitable host cells can be prokaryotic, including bacterial cells such as E. coli, B. subtilis and other suitable bacteria (e.g., Streptococci) or eukaryotic, such as fungal or yeast cells (e.g., Pichia pastoris, Aspergillus species, Saccharomyces cerevisiae, Schizosaccharomyces pombe, Neurospora crassa), or other lower eukaryotic cells, and cells of higher eukaryotes, such as those from insects (e.g., Sf9 insect cells) or mammals, including humans (e.g., Chinese hamster ovary cells (CHO), COS cells, HuT 78 cells, 293 cells, hematopoietic cell lines, and the like), including cell lines and primary cells. See, e.g., Ausubel et al., 1993.

Host cells which produce recombinant polypeptide or a variant thereof can be produced as follows. A nucleic acid (e.g., DNA) encoding the polypeptide is inserted into a nucleic acid vector, e.g., a DNA vector, such as a plasmid, cosmid, phage, virus or other suitable replicon for expression. The resulting vector is introduced into a host cell, using known methods, and the host cell is maintained under conditions appropriate for growth of the host cell and expression of the endogenous DNA. For example, a nucleic acid encoding the polypeptide or a variant thereof can be incorporated into a vector, operably linked to one or more expression control elements, and the construct can be introduced into host cells, which are maintained under conditions suitable for expression of the polypeptide. The construct can be introduced into cells by a method appropriate to the host cell selected (e.g., transformation, transfection, electroporation, or infection). The encoded polypeptide can be isolated from the host cells or medium.

A variety of vectors is available, including vectors which are maintained in single copy or multiple copy, or which become integrated into the host cell chromosome. Suitable expression vectors can contain a number of components, including, but not limited to one or more of the following: an origin of replication; a selectable marker gene; one or more expression control elements, such as a transcriptional control element (e.g., a promoter, an enhancer, terminator), and/or one or more translation signals; a signal sequence or leader sequence for membrane targeting or secretion (of mammalian origin or from a heterologous mammal or non-mammalian species).

The present invention also relates to antibodies, both polyclonal and monoclonal, which bind the polypeptides of the invention in vitro and/or in vivo and, optionally, inhibit an activity or function characteristic of the polypeptide. The present invention further relates to pharmaceutical compositions which comprise anti-polypeptide antibodies and a suitable carrier, such as a buffer; they can also include further components, such as stabilizers. The antibodies of the present invention are useful in a variety of applications, including separation techniques, research, diagnostic and therapeutic applications.

In addition, antibodies of the present invention can be used to detect and/or measure the level of the polypeptide in a sample (e.g., tissue or body fluid) obtained from an individual (e.g., a human). For example, a sample (e.g., tissue and/or fluid) can be obtained from an individual and a suitable immunological method can be used to detect and/or measure polypeptide levels.

In an application of the method, antibodies which bind the polypeptide are used to analyze tissues or cells in mammals for reactivity and/or expression (e.g., immunohistologically). Thus, the antibodies of the present invention are useful in immunological diagnostic methods of assessing expression of the polypeptide in normal tissues or cells and cancerous tissues or cells.

Anti-polypeptide antibodies also have therapeutic uses. An anti-polypeptide antibody can be administered in an amount effective to inhibit the activity of the polypeptide. For therapy, an effective amount is sufficient to achieve the desired therapeutic and/or prophylactic effect. The antibody can be administered in a single dose or multiple doses. The dosage can be determined by methods known in the art and is dependent, for example, upon the individual's age, sensitivity, tolerance and overall well-being. Suitable dosages for antibodies can be from 0.1-1.0 mg/kg body weight per treatment.

According to the method, an antibody can be administered to an individual (e.g., a human) alone or in conjunction with another agent, which is administered before, along with or subsequent to administration of the antibody.

Compositions of the present invention can be administered by a variety of routes, including, but not limited to, parenteral (e.g., injection, including but not limited to, intravenous, intraarterial, intramuscular, subcutaneous; inhalation, including but not limited to, intrabronchial, intranasal or oral inhalation, intranasal drops; topical) and non-parenteral (e.g., oral, including but not limited to, dietary; rectal).

The formulation used will vary according to the route of administration selected (e.g., solution, emulsion, capsule). An appropriate composition comprising the nucleic acids, proteins or antibodies to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. See, generally, Remington's Pharmaceutical Science, 16th Edition, Mack, Ed. (1980). For inhalation, the compound can be solubilized and loaded into a suitable dispenser for administration (e.g., an atomizer, nebulizer or pressurized aerosol dispenser). Nucleic acids, proteins and antibodies can be administered individually, together or in combination with other drugs or agents (e.g., other chemotherapeutic agents, immune system enhancers).

Methods for Identifying Differentially Expressed Transcripts

The invention in its broadest sense provides a method to identify genes associated with a particular phenotype, e.g., disease or malignancy, by identifying genes that are differentially expressed and so are associated with the phenotype. Methods for identifying differentially expressed transcripts, transcripts which differ in abundance between samples being compared, are known to the art.

The technical approaches used to compare RNA samples in vitro and identify differentially expressed transcripts, though being extremely diverse, all stem from the same origin: the method of differential hybridization or differential screening. Differential screening involves checking sequences (clones), picked at random from two libraries, for presence in one of the original RNA samples in higher concentration than in another. An enhancement of differential screening, called subtractive hybridization, enriches samples for differentially expressed transcripts prior to differential screening. Exemplary subtractive hybridization methods are described hereinbelow, although any other methods which identify differentially expressed genes may be employed to identify the nucleic acid molecules of the invention (e.g., see U.S. Pat. Nos. 5,726,022, 5,827,658, 5,958,738 and 6,066,457 and Matz et al., 1998).

For example, an alternative approach to differential screening is ‘differential display’ (DD). The basis of DD is as follows: (i) simplified pools of cDNA fragments (called subsets) are produced from the RNA samples being compared, their content being strictly defined by the intrinsic features of the protocol; (ii) the analogous pools obtained from the compared RNA samples are resolved side by side on a polyacrylamide gel (the produced band patterns are called fingerprints); (iii) fragments (defined by length) that are present in only one sample (or much more abundant in one than in the other) are excised from the gel and investigated; (iv) by changing the parameters of the pool generation protocol, other pools are produced and studied in a similar way to compare more mRNAs. The advantage of such an approach is that the abundance of several dozen randomly picked cDNA fragments can be checked simultaneously. Another important feature of DD is the ability to compare more than two RNA samples at once.

The methods of the invention may be employed with any source of nucleic acid in which one is interested in comparing for differences. The sources may be eukaryotic, prokaryotic, invertebrate, vertebrate, mammalian, non-mammalian, plant and others. Preferably, the source is a vertebrate source such as a mammalian source, e.g., from canine, bovine, murine, ovine, caprine, equine, and primates such as humans. RNA may be isolated by any known means as a subset of the genomic nucleic acid and subsequent synthesis of cDNA. It is also desirable to use cDNA as the first nucleic acid sample in assays in which cDNA or RNA is used as the second nucleic acid sample to prevent the isolation of products that are derived from intronic genomic sequences.

One subtractive hybridization method employs first and second nucleic acid samples that are separately subjected to at least one restriction endonuclease. The restriction endonuclease may provide for blunt ends or staggered (sticky) ends, usually staggered ends. It is preferred that both first and second nucleic acid samples are subjected to the same restriction endonuclease and that such endonuclease is one which recognizes and cuts at a four base site. For the subsequent steps it is further preferred that such restriction endonuclease be one which recognizes a four base sequence found within a longer six base or eight base sequence recognized by a restriction endonuclease. Almost 1500 restriction endonucleases are now known and at least 150 are commercially available. Complete lists plus details of restriction sites and reaction conditions are published, for example in Brown, 1991.

Once the first and second nucleic acid samples have been separately fragmented to produce first and second nucleic acid sample fragments, double-stranded oligonucleotide adaptors are ligated onto the ends of each of the strands of the fragments. The adaptor will usually be staggered at both ends, with one strand being longer than the other. The adaptors will generally serve to provide the sequence complementary to a primer to be used when a subsequent amplification step is employed. Thus, typically one end of the adaptor will be double-stranded and have one end complementary to the ends of the double-stranded nucleic acid fragments from the digestion, sometimes referred to herein as the proximal end of the adaptor. Each adaptor will preferably further contain a restriction site located distal to the proximal end.

The restriction site in the adaptor is preferably one which has a six or eight base consensus sequence, and most preferably is such a one that further contains a 3′ sequence that ends in a four base consensus sequence that has ends that are complementary to the same ends that are created by the six or eight base cutter that is adjacent and external to it. Examples of such restriction endonucleases include, but are not limited to, DpnII (′GATC); BglII (A′GATCT); BamHI (G′GATCC); Tsp509I (′AATT), EcoRI (G′AATTC) and PacI (TT′AATTAA).

It is preferred that the adaptors used for the first nucleic acid sample fragments contain a restriction site which is different than the one used in the adaptors for the second nucleic acid sample fragments. The adaptor may further optionally contain a ligand binding end. A ligand binding end is particularly important if the fragments will not be amplified. A ligand or ligand binding end is a molecule which binds to another molecule and so permits the physical or chemical separation of molecules which are physically linked to the ligand or ligand binding end, e.g., biotin binds to avidin or streptavidin and digoxigenin binds to anti-digoxigenin antibodies. It is preferred that the adaptor have one strand longer than the other to serve as a complement to primers if the fragments are to be amplified.

For example, in one embodiment only the first nucleic acid sample fragments will contain adaptors having a restriction site. The second nucleic acid sample fragments do not necessarily need to have adaptors or a primer used for amplification with a restriction site. If this embodiment is employed, the adaptors and/or primers for the second nucleic acid sample fragments will have a ligand binding moiety to enable capture of the second nucleic acid sample fragments.

Alternatively, it is possible to ligate the same adaptors onto nucleic acid of the first nucleic acid sample and the second nucleic acid sample if different primers are subsequently used to amplify the two sample populations so long as a restriction endonuclease site is encoded within the primers used to amplify the first nucleic acid sample fragments.

Additionally, the adaptor ligated onto the first nucleic acid sample fragments may have “non-nested” restriction endonuclease sites; e.g. 5′EcoRI-GATC3′, where the EcoRI site is external to an initial DpnII digestion site. This protocol is less preferred, however, because when the EcoRI site is subsequently targeted by the restriction endonuclease in order to release the homoduplex from its biotinylated adaptors, approximately 1/16 of the cDNA molecules may contain an internal EcoRI site.

The first and second nucleic acid fragments may be separately amplified to enhance the assay, preferably by PCR or other methods, using primers containing a sequence complementary to the respective adaptors and a ligand binding end.

Thus, the second nucleic acid sample fragments and the first nucleic acid sample fragments may be amplified separately by adding appropriate primers complementary to the adaptors using PCR, typically for about 10-35 cycles, more typically about 20 cycles, depending upon the initial concentration of second or first nucleic acid sample fragments being amplified. For a general overview of PCR, see Innis et al., 1990; and U.S. Pat. Nos. 4,683,195 and 4,683,202. The adaptors do not need to be removed.

The amplified first and second nucleic acid sample fragments are combined under hybridization conditions such that the fragments hybridize together creating essentially several possible complexes: first nucleic acid/second nucleic acid matches, second nucleic acid/second nucleic acid matches, and first nucleic acid/first nucleic acid matches. It is preferred that the second nucleic acid fragments are present in excess of the first nucleic acid sample fragments to increase the probability that the first nucleic acid/first nucleic acid complexes are representative of nucleic acid not found in the second nucleic acid sample. For example, second nucleic acid sample fragments are combined with the adaptor-ligated first nucleic acid fragments, with the second nucleic acid sample fragments present in excess, usually at least 5-fold excess and less than 500-fold excess, preferably about 100-fold excess for the first cycle of hybridization. Hybridization will be allowed to proceed at high stringency temperatures, usually about 60°-70° C. Various buffers and salt concentrations may be used to adjust for the desired stringency as will be appreciated by those in the art.

The first nucleic acid/first nucleic acid complexes present in the combined first and second nucleic acid solution can be readily separated from the other complexes depending upon the ligand used. Most conveniently, all of the combined fragments will be subjected to a restriction enzyme which recognizes the site in the first nucleic acid sample adaptors which will effectively remove the ligand binding end from the first nucleic acid/first nucleic acid molecules and not from the others. Thus, by capture technology which will attract the ligand binding end of the second nucleic acid/first nucleic acid complexes, one can readily separate out the first nucleic acid/first nucleic acid complexes. The first nucleic acid/first nucleic acid complexes may be further amplified and isolated, by, for example, ligating new adaptors onto the ends of the molecules and amplifying by PCR.

It may be of interest to carry out the process more than once, where different restriction endonucleases are used. Different fragments may be obtained and result in additional information.

Any resulting unique first nucleic acid sequences (i.e. those not found in the second nucleic acid sample) can be used as probes to identify sites in the first nucleic acid sample which differ from the second nucleic acid source. For this purpose they may be labeled in a variety of ways. Desirably in order to obtain substantially homogeneous compositions of each of the first nucleic acid sample sequences, the first nucleic acid sample sequences may be cloned by inserting into an appropriate cloning vector for cloning in a prokaryotic host. If desired, the cloned DNA may be sequenced to determine the nature of the target DNA. Alternatively, the cloned DNA may be labeled and used as probes to identify fragments in libraries carrying the target DNA. The target DNA may be used to identify the differences which may be present between the two sources of nucleic acid.

The resulting target DNA will be greatly enriched. It may be used as a probe to identify sites on the first nucleic acid sample sequences which differ from the second nucleic acid. The target nucleic acid may be sequenced directly by PCR or it may be cloned by inserting it in a cloning vector for cloning into a host cell. The cloned DNA can be sequenced to determine the nature of the target DNA through the use of dot blotting or other procedure. It may also be labeled and used as probes to identify fragments in libraries carrying the target DNA. Sequences can be identified and cloned for sequencing. Comparative searches with sequences described in accessible libraries such as Genbank (National Center for Biotechnology Information, Natl. Library of Medicine, National Institutes of Health, 8600 Rockville Pike, Bethesda, Md. 20894); Protein Identification Resource (PIR, Natl. Biomedical Research Foundation, 3900 Reservoir Road NW, Washington, D.C. 20007; EMBL, European Molecular Biology Laboratory, Heidelberg, Germany) can aid in identifying the sequence.

In another substractive hybridization method, the genes are isolated from tumorigenic tissue or cells having a malignant or neoplastic phenotype, as well as from normal cells. Thus, the method entails substantive hybridization of two sets of cDNA, one provided from the tissue of interest and the other from normal tissue.

Any technique may be employed to produce the cDNAs, including any methods of purifying total RNA or mRNA and any primers. For example, primers for first strand synthesis can include polyT, “anchored” polyT, primers with restriction enzyme recognition sequences build-in, primers to complement a polyN tail created by the terminal transferase, where N stands for any one of the four nucleotides to be used. The polymerases can be any of the available and known polymerases. Either set of cDNAs could be maintained and amplified by cloning into a plasmid. A set of cDNAs from the normal tissue can serve as subtractor cDNA for annealing to cDNA from samples from multiple tissues of interest. Further variations and options would be readily apparent to one skilled in the art.

In a preferred embodiment, a double stranded adapter oligonucleotide (oligo) is attached to the ends of the cDNA set prepared from the tissue of interest. The oligo set is composed of two, at least partially complementary synthetic oligos. They are attached to the cDNA by a DNA ligase. The attachment can be preceded by creation of protruding ends on the cDNA by cleavage with a restriction endonuclease for which a recognition side was built-in on the cDNA ends by the choice of oligos used to create the cDNA set. In this embodiment, the oligo set to be attached is designed to create, after self annealing, complementary ends to the cDNA. Alternatively, the cDNA is made blunt-ended by enzymatic reaction, Klenow fragment by example. Then the oligo set would be ligated to the cDNA by a blunt-ended ligation.

In a preferred embodiment, the cDNA ends are made blunt-ended as described above and ligated to an adapter set which is blunt ended at least at one end. Preferably, the cDNA derived from the normal tissue is biotinylated. Again, this requirement can be achieved by any of a number of methods readily apparent to one skilled in the art. By way of example, but not limited to those examples, the biotin label can be incorporated into the cDNA starting with the synthesis of a second strand or can result from PCR amplification of a pre-made cDNA set. The label can also be introduced by PCR amplification or by “nicktranslation” of a cDNA set or by photobiotinylation.

The invention also includes a mixing of the two cDNA sets, derived from the tissue of interest and from the normal tissues, followed by denaturation and annealing. An excess of subtractor cDNA will increase the efficiency of annealing (and eventual removal, see below) of the sequences that are common to the two cDNA sets and are not present in the tissue of interest. The melting and annealing conditions are standard for such experiments and known to one skilled in the art. The annealing results in populations of hybrid cDNAs.

Magnetic spheres make the job of removal of biotin containing DNA easier. Thus, little biotin labeled DNA should escape untrapped, reducing the background levels of cDNA recovered from the subtractive hybridization. Streptavidin coated beads are available commercially. They are used by other to remove biotin labeled DNA, unlike the current disclosure that employs them within in a subtractive hybridization protocol.

The subtractive hybridization results in a cDNA fraction, hereafter called flow-through, enriched in cDNAs representing genes expressed in tumorigenic tissue or cells having a malignant to neoplastic phenotype, but not free of all other cDNAs. Initial analysis is sometimes facilitated by cloning the cDNAs of the flow-through. The cloning step itself is facilitated by first carrying out a PCR amplification of the flow-through cDNAs. Both of these steps can be accomplished by use of the previously described adapter set that can a) contain a restriction enzyme recognition site and b) one of the oligos in the set can be used as PCR primer. All these analysis employ standard molecular biology techniques and numerous options and shortcuts will be readily apparent to one skilled in the art. In either or both case, cloned flow-through cDNAs or direct sequencing, “single lane” dideoxy sequencing may suffice if the sequence is known. Sequencing reactions could employ as primer the same oligo described above as part of the oligo set.

Another analysis would be either a RT-PCR or Northern blot analysis. The chosen cloned cDNA(s) described above would be hybridized to equivalent amounts of nucleic acids, DNA and/or RNA, from both the tissue of interest and the normal tissue. The relative intensity of the bands would be compared spectrophotometrically and result in a estimate of copy number. To a person skilled in the art, variations and shortcuts will be readily apparent. For example, but not limited to this examples, one could use dot blots rather than gels and blotting, or one can incorporate a control hybridization with a probe not expected to hybridize to the gene, to standardize the amount of nucleic acids from the two tissues used.

The invention will be further described by the following non-limiting example.

### EXAMPLE

Cord blood (CB) CD34⁺ cells (Zhao et al., 1999) were transduced with a MSCV-retrovirus vector containing either eGFP alone (eGFP) or BCR/ABL cDNA-IRES-eGFP (p210-eGFP). GFP⁺ cells were FACS selected (>90% purity), and subtractive hybridization performed between the two populations using the Clontech PCR-Select™ System, according to the manufacturer's recommendations.

The PCR-Select method requires only one round of subtractive hybridization to subtract and equalize cDNAs, combined with suppression PCR for efficient amplification of target molecules. This dramatically increases the probability of obtaining differentially expressed, rare transcripts. In contrast, traditional subtractive hybridization methods require a large amount of poly A⁺ RNA and the tedious separation of ss and ds cDNA fractions. For use as tester and driver, cDNA is synthesized from only 0.5-2.0 μg of poly A⁺ RNA prepared from the two types of tissue or cells under comparison.

A total of 79 subtracted cDNA clones were expressed in p210-eGFP⁺ but not eGFP⁺ transduced CD34⁺ cells. The cDNAs were between 400-900 bp in length. Forty-one of the cDNA sequences represent uncharacterized human proteins. Of these, at least 15 were closely homologous to expressed sequence tags (ESTs), at least 10 had homology to genomic clones for which no mRNA transcripts or proteins had been identified, while 1 sequence had no match in available genomic, RNA or protein databases. In addition, at least 22 sequences were identified that have significant homologies to known genes, which are involved in protein degradation, signal transduction, cell cycle regulation, and in RNA splicing. The observation that 4 differentially expressed genes (SRPK1, Sty, Gu, SNRNP-G) are involved in RNA splicing may provide an explanation for the finding that CML CD34⁺ cells and cell lines express a number of alternatively spliced proteins, including Pyk2, beta₁-B integrin, CSCP (chondroitin sulfate core protein), and MPP1 (human palmitoylated erythrocyte membrane protein).

The identified sequences and/or their full length genes are used as probes to detect expression patterns in normal and CML cells, and/or cloned into expression vectors. The identified sequences and/or their full length equivalent may also be used to prepare primers for RT-PCR analysis of the expression of these sequences in primary CML cells. Thus, gene expression analyses will lead to important new insights in the molecular mechanisms underlying CML and may identify critical targets for novel therapies for this disease.

### REFERENCES

Bartram et al., Nature, 306:277-280 (1983).

Berger and Kimmel, Guide to Molecular Cloning Techniques, Methods in Enzymology 152 Academic Press, Inc., San Diego, Calif.

Bhatia, R., Verfaillie, C. M., Miller, J. S. and McGlave, P. B., Blood 89:2623-2634 (1997).

Bolli et al., Nucleic acids Res., 24:4660-4667 (1996).

Brown, Molecular Biology Labfax, BIOS, Oxford (1991).

Chomczynski et al., Anal Biochem, 162:156-9 (1987).

Chung and Wong, Oncogene, 10:1261-1268 (1995).

Clarkson, B. D. et al., Leukemia, 11:1404-1428 (1997).

Clarkson and Strife, Leukemia, 7:1683-1721 (1993).

Cotter, T. G., Leuk. Lymphoma, 1:231-244 (1995).

Current Protocols in Molecular Biology, F. M. Ausubel et al., eds., Current Protocols, a joint venture between Greene Publishing Associates, Inc. and John Wiley & Sons, Inc., (1994 Supplement).

Daley and Ben Neriah, Adv. Cancer Res. 57:151-184 (1991).

Daley, G. Q., Van Etten, R. A. and Baltimore, D., Science, 247:824-829 (1990).

Darnell et al., Molecular Cell Biology, 2nd Ed., W. H. Freeman and Co., New York (1990).

Deininger et al., Cancer Res., 60:2049-2055 (2000).

Deisseroth et al., Blood, 83:3068-3076 (1994).

Deisseroth and Arlinghaus, eds. Chronic Myelogenous Leukemia-Molecular Approaches to Research and Therapy, New York, Marcel Dekker (1991).

Ehrlich, ed., PCR Technology, W. H. Freeman and Company, N.Y. (1991).

Enright, H. and McGlave, P. B., Oncology, 11:1295-1300 (1997).

Hames and Higgins, eds.: Nucleic Acid Hybridization: A practical Approach, IRL Press, 1985.

Innis et al., PCR Protocols: A Guide to Methods and Applications, Academic Press, San Diego (1990).

Kantarjian, H. M. et al., Blood, 66:1326-1335 (1985).

Kerr and Sadowski, J. Biol. Chem., 247:311-318 (1989).

Konopka et al., Cell, 37:1035-1042 (1984).

Lisitsyn et al., Science, 259:946 (1993).

Matz et al., Nucl. Acids Res., 26:5537-5543 (1998).

McWhirter et al., Mol. Cell Bio., 11:1553-1565 (1991).

Meinkoth and Wahl, Analytical Biochemistry, 238:267-284, (1984).

Needleman and Wunsch, J. Mol. Biol. 48: 443 (1970).

Nielsen et al., Science 254:1497-1500 (1991).

Nikiforov et al., PCR Meth. & App., 3:285-291 (1994).

Pearson and Lipman, Proc. Natl. Acad. Sci. (U.S.A.) 85: 2444 (1988).

Pichert, G. et al., Blood, 84:2109-2114 (1994).

Saiki et al., Science, 239:487-491 (1988).

Sambrook et al., Molecular Cloning—A Laboratory Manual (2nd ed.) Vol. 1-3, Cold Spring Harbor Laboratory, Cold Spring Harbor Press, N.Y. (1989).

Sawyers et al., Cell, 64:337-350 (1991).

Smith and Waterman, Adv. Appl. Math. 2: 482 (1981).

Verfaillie, C. M., Hematol. Oncol. Clin. North Am., 12:1-30 (1998).

Wetmur and Sninsky in “PCR Strategies” ed. Innis et al., Academic Press pp.69-83 (1995).

Zhao et al., Blood, 90:4687-4686 (1999).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to identify a population of nucleic acid molecules present in one nucleic acid sample and not in another nucleic acid sample, comprising:
a) contacting nucleic acid from a first sample with nucleic acid from a second sample under hybridization conditions so as to form binary complexes, wherein the first sample comprises nucleic acid from cells which express a protein that is associated with hematopoietic cell malignancy and wherein the second sample comprises nucleic acid from cells which do not express the protein; and
b) identifying a population of nucleic acid molecules present in one of the samples and not in the other sample.

2. The method of claim 1 wherein the nucleic acid molecules which are identified are present in the first sample and not in the second sample.

3. The method of claim 1 wherein the nucleic acid molecules which are identified are present in the second sample and not in the first sample.

4. The method of claim 1 further comprising isolating at least one of the identified nucleic acid molecules.

5. The method of claim 1 wherein the cells are CD34⁺ cells.

6. The method of claim 1 wherein the first sample comprises nucleic acid from cells which express a chimeric protein that is associated with the malignancy.

7. The method of claim 6 wherein the chimeric protein is p210^{BCR-ABL}.

8. The method of claim 6 wherein the chimeric protein is p185^{BCR-ABL}.

9. The method of claim 6 wherein the chimeric protein is any one of p230^{BCR/ABL}, TEL-ABL, PDGF-ABL, AML-ETO, PML-RARα, or MDS/EV1.

10. The method of claim 1 wherein the first sample comprises nucleic acid from cells which are augmented with an expression cassette comprising a first nucleic acid segment encoding a protein that is associated with the malignancy.

11. The method of claim 10 wherein the expression cassette further comprises a second nucleic acid segment comprising a marker gene.

12. The method of claim 10 wherein the marker gene is a drug resistance gene.

13. The method of claim 11 wherein the marker gene encodes a fluorescent protein.

14. The method of claim 13 wherein the marker gene encodes a green fluorescent protein.

15. The method of claim 11 wherein the second sample comprises nucleic acid from cells which are augmented with an expression cassette comprising a second nucleic acid segment comprising the marker gene.

16. The method of claim 1 wherein the identified population includes a nucleic acid molecule comprising an open reading frame comprising any one of SEQ ID NOs:1-79.

17. An isolated nucleic acid molecule, the expression of which is increased in p210^{BCR-ABL}-expressing cells, comprising an open reading frame comprising any one of SEQ ID NOs: 1, 3-4, 11, 14-17, 19, 22, 25, 27, 29, 32, 35, 37-39, 41, 44-47, 52-54, 57-58, 60-62, 64, 67-79, or the complement thereof.

18. An isolated nucleic acid molecule which hybridizes under moderate stringency conditions to any one of SEQ ID NOs: 1, 3-4, 11, 14-17, 19, 22, 25, 27, 29, 32, 35, 37-39, 41, 44-47, 52-54, 57-58, 60-62, 64, 67-79, or the complement thereof.

19. The isolated nucleic acid molecule of claim 17 or 18 which consists of any one of SEQ ID NOs: 1, 3-4, 11, 14-17, 19, 22, 25, 27, 29, 32, 35, 37-39, 41, 44-47, 52-54, 57-58, 60-62, 64, or 67-79.

20. An expression cassette comprising an open reading frame comprising any one of SEQ ID NOs: 1, 3-4, 11, 14-17, 19, 22, 25, 27, 29, 32, 35, 37-39, 41, 44-47, 52-54, 57-58, 60-62, 64, 67-79, or the complement thereof, operably linked to a promoter functional in a host cell.

21. An expression cassette comprising an open reading frame comprising a nucleic acid sequence which hybridizes under moderate stringency conditions to any one of SEQ ID NOs: 1, 3-4, 11, 14-17, 19, 22, 25, 27, 29, 32, 35, 37-39, 41, 44-47, 52-54, 57-58, 60-62, 64, 67-79, or the complement thereof, operably linked to a promoter functional in a host cell.

22. A host cell, the genome of which is augmented with the expression cassette of claim 21.

23. A method to detect a gene, comprising:
a) contacting a eukaryotic nucleic acid sample with a probe comprising any one of SEQ ID NOs:1, 3-4, 11, 14-17, 19, 22, 25, 27, 29, 32, 35, 37-39, 41, 44-47, 52-54, 57-58, 60-62, 64, 67-79, or the complement thereof, or a portion thereof, under hybridization conditions so as to form a complex; and
b) detecting or determining complex formation.

24. A method to detect a gene, comprising:
a) contacting a eukaryotic nucleic acid sample with at least one oligonucleotide specific for any one of SEQ ID NOs: 1, 34, 11, 14-17, 19, 22, 25, 27, 29, 32, 35, 37-39, 41, 44-47, 52-54, 57-58, 60-62, 64, or 67-79 under conditions so as to yield an amplified product; and
b) detecting an amplified product corresponding to SEQ ID NOs: 1, 3-4, 11, 14-17, 19, 22, 25, 27, 29, 32, 35, 37-39, 41, 44-47, 52-54, 57-58, 60-62, 64, or 67-79.

25. The method of claim 23 wherein the amount of complex formation is compared to the amount of complex formation in a control sample.

26. The method of claim 24 wherein the amount or presence of the amplified product is compared to the amount or presence of the amplified product in a control sample.

27. The method of claim 23 or 24 wherein the sample is a RNA sample.

28. The method of claim 23 or 24 wherein the sample is genomic DNA.

29. The method of claim 23 or 24 wherein the sample is a cDNA sample.

30. An isolated polypeptide encoded by the nucleic acid molecule of claim 17 or 18.

31. A method to identify an agent that interacts with a polypeptide, comprising:
a) contacting a polypeptide encoded by a nucleic acid molecule comprising an open reading frame comprising any one of SEQ ID NOs:1-79 with the agent; and
b) detecting an determining whether the agent interacts with the polypeptide.

32. A method to inhibit expression of a gene, comprising:
contacting a eukaryotic cell with an amount of a nucleic acid molecule comprising any one of SEQ ID Nos:1-79, the complement thereof, a portion thereof, or a nucleic acid molecule which hybridizes thereto under stringent hybridization conditions, effective to inhibit expression of the corresponding gene in the eukaryotic cell.

33. The method of claim 31 or 32 wherein the nucleic acid molecule comprises any one of SEQ ID Nos. 1, 3-4, 11, 14-17, 19, 22, 25, 27, 29, 32, 35, 37-39, 41, 44-47, 52-54, 57-58, 60-62, 64, or 67-79.

34. A method to identify an agent that inhibits or reduces the expression of a gene associated with hematopoietic cell malignancy, comprising:
(a) containing a cell or an extract thereof with the agent, wherein the cell expresses a nucleic acid molecule comprising any one of SEQ ID NOS: 1-79; and
(b) identifying an agent that inhibits or reduces expression of the nucleic acid molecule or the polypeptide encoded thereby.

35. An agent identified by the method of claim 34.

36. The method of claim 34 wherein the agent is a ribozyme, DNAzyme, antibody or antisense molecule.

37. The method of claim 34 wherein the agent alters cell adhesion, cell migration, cell proliferation, cell death or genetic instability.

38. The method of claim 34 wherein the agent inhibits or reduces cell migration, cell proliferation, cell death or genetic instability.

39. The method of claim 34 wherein the agent increases cell adhesion.
